(19) United States
(12) Patent Application Publication
(10) Pub. No.: US 2014/0213512 A1
(43) Pub. Date: Jul. 31, 2014

(54) Method of Treating or Ameliorating Type 1 Diabetes Using FGF21

(71) Applicant: Amgen Inc., Thousand Oaks, CA (US)

(72) Inventors: Murielle Marie Ellison, Thousand Oaks, CA (US); Shanaka Stanislaus, Thousand Oaks, CA (US); Jing Xu, Thousand Oaks, CA (US)

(73) Assignee: Amgen Inc., Thousand Oaks, CA (US)

(21) Appl. No.: 14/241,848

(22) PCT Filed: Aug. 30, 2012
(86) PCT No.: PCT/US2012/053216

§ 371 Date: Feb. 27, 2014

Related U.S. Application Data
(60) Provisional application No. 61/529,641, filed on Aug. 31, 2011.

(51) Int. Cl. A61K 38/18

(52) U.S. Cl. CPC A61K 38/1825
USPC 514/4.8; 514/9.1; 514/7.3

(57) ABSTRACT

Methods of treating metabolic diseases and disorders using a FGF21 polypeptide are provided. In various embodiments the metabolic disease or disorder is type 1 diabetes, obesity, dyslipidemia, elevated glucose levels, elevated insulin levels, diabetic nephropathy, neuropathy, retinopathy, ischemic heart disease, peripheral vascular disease and cerebrovascular disease

This patent application claims priority benefit of U.S. Provisional Patent Application No. 61/529,641 filed Aug. 31, 2011, each of which is incorporated herein in its entirety.

### FIELD OF THE INVENTION

The disclosed invention relates to the treatment or amelioration of Type 1 Diabetes by administering a therapeutically effective amount of an FGF21 polypeptide or FGF21 variant to a subject in need thereof.

### BACKGROUND OF THE INVENTION

Fibroblast Growth Factor 21 (FGF21) is a secreted polypeptide that belongs to a subfamily of Fibroblast Growth Factors (FGFs) that includes FGF19, FGF21, and FGF23 (Itoh et al., (2004) Trend Genet. 20:563-69). FGF21 is an atypical FGF in that it is heparin independent and functions as a hormone in the regulation of glucose, lipid, and energy metabolism.

It is highly expressed in liver and pancreas and is the only member of the FGF family to be primarily expressed in liver. Transgenic mice overexpressing FGF21 exhibit metabolic phenotypes of slow growth rate, low plasma glucose and triglyceride levels, and an absence of age-associated type 2 diabetes, islet hyperplasia, and obesity. Pharmacological administration of recombinant FGF21 protein in diseased rodent and primate models results in normalized levels of plasma glucose, reduced triglyceride and cholesterol levels, and improved glucose tolerance and insulin sensitivity. In addition, FGF21 reduces body weight and body fat by increasing energy expenditure, physical activity, and metabolic rate. Experimental research provides support for the pharmacological administration of FGF21 for the treatment of type 2 diabetes, obesity, dyslipidemia, and other metabolic conditions or disorders in humans.

Two major types of diabetes, type 1 and type 2 have been defined. In type 1 diabetes, also called insulin dependent diabetes mellitus (IDDM), the pancreas produces insufficient levels of insulin. Patients suffering from type 1 diabetes must rely on administered insulin to survive. Patients suffering from type 2 diabetes, also referred to as non-insulin dependent diabetes mellitus (NIDDM), can still produce insulin, but in a relatively inadequate manner. In many cases the pancreas produces larger quantities of insulin than normal. A distinguishing feature of type 2 diabetes is a lack of sensitivity to insulin by the cells of the body (particularly fat and muscle cells).

In addition to the problems of increased insulin resistance, the release of insulin by the pancreas may also be defective and suboptimal in patients suffering from type 2 diabetes. In fact, it is known that there is a steady decline in beta cell production of insulin in type 2 diabetes that contributes to worsening glucose control; this is a major factor for many patients with type 2 diabetes who ultimately require insulin therapy. Furthermore, the livers of type 2 diabetes patients continue to produce glucose through gluconeogenesis, despite elevated glucose levels. Thus, in type 2 diabetes patients the control of gluconeogenesis can become compromised.

A patient suffering from type 1 diabetes needs insulin to survive (see, e.g., Falorni et al., (1995) Bailliere's Clin. Endocrinol. Met. 9:25-46). Insulin can be used to treat both type 1 and type 2 diabetes but no other current compound on the market used to treat type 2 diabetes can be used to treat type 1 diabetes (Raslova, (2010) Vasc. Health Risk Manag. 6:399-410). In contrast to established insulin therapy, the present disclosure provides a method of treating Type 1 Diabetes using FGF21, and thus a therapeutic alternative for health care professionals treating type 1 diabetes patients.

### SUMMARY OF THE INVENTION

In one aspect a method of treating a metabolic disorder is provided. In one embodiment the method comprises administering to a subject in need thereof a therapeutically effective amount of (a) a human FGF21 polypeptide; or (b) a FGF21 variant polypeptide. In a further embodiment the metabolic disorder is type 1 diabetes. In a further embodiment the metabolic disorder is dyslipidemia. In a further embodiment the metabolic disorder is obesity. In a further embodiment the metabolic disorder is diabetic nephropathy. In a further embodiment the metabolic disorder comprises a condition in which the subject has a fasting blood glucose level of greater than or equal to 100 mg/dL. In one embodiment the subject on which the method is performed is a mammal and in another the mammal is a human. In a specific embodiment the human FGF21 polypeptide comprises one of SEQ ID NOs:4 and 8 and in another embodiment the human FGF21 polypeptide is encoded by one of SEQ ID NOs:3 and 7. In still a further embodiment the FGF21 variant comprises one or more mutations in the mature FGF21 sequence of one of SEQ ID NOs:4 and 8 selected from the mutations presented in Tables 1-13. In another embodiment the FGF21 polypeptide is administered in the form of a pharmaceutical composition comprising the FGF21 polypeptide in admixture with a pharmaceutically-acceptable carrier. In yet a further embodiment the disclosed method further comprises the step of determining the subject's blood glucose level at a timepoint subsequent to the administration. In another embodiment the method further comprises the step of determining the subject's serum insulin level at a timepoint subsequent to the administration. In still another embodiment the human FGF21 polypeptide or human FGF21 variant polypeptide further comprises one or more of (a) one or more PEG molecules; and (b) an Fc polypeptide. In a particular embodiment the isolated human FGF21 polypeptide or FGF21 variant polypeptide comprises one of SEQ ID NOs:10 and 12 and in another embodiment the isolated human FGF21 polypeptide; or FGF21 variant polypeptide comprises one of SEQ ID NOs:39 and 41.

Also provided herein is another method of treating a metabolic disorder. In one embodiment the method comprises administering to a subject in need thereof a therapeutically effective amount of a human FGF21 polypeptide comprising an amino acid sequence that has at least 90% sequence identity with one of SEQ ID NOs:4 and 8. In a further embodiment the metabolic disorder is type 1 diabetes. In a further embodiment the metabolic disorder is dyslipidemia. In a further embodiment the metabolic disorder is obesity. In a further embodiment the metabolic disorder is diabetic nephropathy. In a further embodiment the metabolic disorder comprises a condition in which the subject has a fasting blood glucose level of greater than or equal to 100 mg/dL. In one embodiment the subject on which the method is performed is a mammal and in another the mammal is a human. In a specific embodiment the human FGF21 polypeptide comprises one of SEQ ID NOs:4 and 8 and in another embodiment the human FGF21 polypeptide is encoded by one of SEQ ID NOs:3 and 7. In still a further embodiment the FGF21 variant comprises one or more mutations in the mature FGF21 sequence of SEQ ID NO:4 or SEQ ID NO:8 selected from the mutations presented in Tables 1-13. In another embodiment the FGF21 polypeptide is administered in the form of a pharmaceutical composition comprising the FGF21 polypeptide in admixture with a pharmaceutically-acceptable carrier. In yet a further embodiment the disclosed method further comprises the step of determining the subject's blood glucose level at a timepoint subsequent to the administration. In another embodiment the method further comprises the step of determining the subject's serum insulin level at a timepoint subsequent to the administration. In still another embodiment the human FGF21 polypeptide or human FGF21 variant polypeptide further comprises one or more of (a) one or more PEG molecules; and (b) an Fc polypeptide.

### BRIEF DESCRIPTION OF THE DRAWINGS

FIG. 1 is a plot showing the plasma glucose levels measured in streptozotocin-induced type 1 diabetic mice which were administered vehicle, insulin (5 IU/kg), human FGF21 (1 mg/kg), or a combination treatment of insulin (5 IU/kg) and human FGF21 (1 mg/kg); blood glucose was measured on day 3 after treatment initiation, and at 1 hour and 4 hours after the morning injection and on day 5, at 1 hour after the morning injection.

FIG. 2 is a bar graph showing the clinical chemistry analysis of plasma glucose levels measured in streptozotocin-induced type 1 diabetic mice which were administered vehicle, insulin (5 IU/kg), human FGF21 (1 mg/kg), or a combination treatment of insulin (5 IU/kg) and human FGF21 (1 mg/kg); plasma from blood samples was collected prior to treatment (Day 0) and approximately 2 hours post the morning injection (Day 5) were tested.

FIG. 3 is a bar graph showing the clinical chemistry analysis of plasma triglyceride levels measured in streptozotocin-induced type 1 diabetic mice which were administered vehicle, insulin (5 IU/kg), human FGF21 (1 mg/kg), or a combination treatment of insulin (5 IU/kg) and human FGF21 (1 mg/kg); plasma from blood samples collected prior to treatment (Day 0) and approximately 2 hours post the morning injection (Day 5) were tested.

FIG. 4 is a bar graph showing the clinical chemistry analysis of plasma total cholesterol levels measured in streptozotocin-induced type 1 diabetic mice which were administered vehicle, insulin (5 IU/kg), human FGF21 (1 mg/kg), or a combination treatment of insulin (5 IU/kg) and human FGF21 (1 mg/kg); plasma from blood samples was collected prior to treatment (Day 0) and approximately 2 hours post the morning injection (Day 5) were tested.

FIG. 5 is a bar graph showing the clinical chemistry analysis of plasma free fatty acid (NEFA) levels measured in streptozotocin-induced type 1 diabetic mice which were administered vehicle, insulin (5 IU/kg), human FGF21 (1 mg/kg), or a combination treatment of insulin (5 IU/kg) and human FGF21 (1 mg/kg); plasma from blood samples was collected prior to treatment (Day 0) and approximately 2 hours post the morning injection (Day 5) were tested.

FIG. 6 is a bar graph showing the insulin levels measured in streptozotocin-induced type 1 diabetic mice which were administered vehicle, insulin (5 IU/kg), human FGF21 (1 mg/kg), or a combination treatment of insulin (5 IU/kg) and human FGF21 (1 mg/kg); plasma from blood samples was collected prior to treatment (Day 0) and approximately 2 hours post the morning injection (Day 5) were tested.

FIG. 7 is a bar graph showing the glucagon levels measured in streptozotocin-induced type 1 diabetic mice administered with vehicle, insulin (5 IU/kg), human FGF21 (1 mg/kg), or a combination treatment of insulin (5 IU/kg) and human FGF21 (1 mg/kg); plasma from blood samples was collected prior to treatment (Day 0) and approximately 2 hours post the morning injection (Day 5) were tested.

FIG. 8 is a plot showing plasma glucose levels measured in streptozotocin-induced type 1 diabetic mice which were administered vehicle or the dual-20 kd PEGylated FGF21 variant (E37C, R77C, P171G) (1 and 5 mg/kg); blood glucose was measured on Day 0 prior to injection and on days 1, 3, 5, and 7.

FIG. 9 is a plot showing plasma glucose levels measured in streptozotocin-induced type 1 diabetic mice which were administered vehicle or the dual-20 kd PEGylated FGF21 variant (E37C, R77C, P171G) (1 mg/kg); blood glucose was measured on Day 0 prior to injection and on Days 2, 6, 10, 14, 18 and 22.

FIG. 10 is a bar graph showing plasma glucose levels measured in streptozotocin-induced type 1 diabetic mice which were administered vehicle or the dual-20 kd PEGylated FGF21 variant (E37C, R77C, P171G) (1 mg/kg) on Day 0 (post fifth STZ injection) and on Day 27 (seven days post last injection of dual-PEGylated human FGF21 variant (E37C, R77C, P171G)).

FIG. 11 is a bar graph showing triglyceride levels measured in streptozotocin-induced type 1 diabetic mice which were administered vehicle or the dual-20 kd PEGylated FGF21 variant (E37C, R77C, P171G) (1 mg/kg) on Day 0 (post fifth STZ injection) and on Day 27 (seven days post last injection of dual-PEGylated human FGF21 variant (E37C, R77C, P171G)).

FIG. 12 is a bar graph showing cholesterol levels measured in streptozotocin-induced type 1 diabetic mice which were administered vehicle or the dual-PEGylated human FGF21 variant (E37C, R77C, P171G) (1 mg/kg) on Day 0 (post fifth STZ injection) and on Day 27 (seven days post last injection of dual-PEGylated human FGF21 variant (E37C, R77C, P171G)).

FIG. 13 is a bar graph showing HDL levels measured in streptozotocin-induced type 1 diabetic mice which were administered vehicle or the dual-PEGylated human FGF21 variant (E37C, R77C, P171G) (1 mg/kg) on Day 0 (post fifth STZ injection) and on Day 27 (seven days post last injection of dual-PEGylated human FGF21 variant (E37C, R77C, P171G)).

FIG. 14 is a bar graph showing NEFA levels measured in streptozotocin-induced type 1 diabetic mice which were administered vehicle or the dual-PEGylated human FGF21 variant (E37C, R77C, P171G) (1 mg/kg) on Day 0 (post fifth STZ injection) and on Day 27 (seven days post last injection of dual-PEGylated human FGF21 variant (E37C, R77C, P171G)).

FIG. 15 is a bar graph showing insulin levels measured in streptozotocin-induced type 1 diabetic mice which were administered vehicle or the dual-PEGylated human FGF21 variant (E37C, R77C, P171G) (1 mg/kg) on Day 0 (post fifth STZ injection) and on Day 27 (seven days post last injection of dual-PEGylated human FGF21 variant (E37C, R77C, P171G)).

FIG. 16 is a plot showing the change in body weight measured in streptozotocin-induced type 1 diabetic mice, which were administered vehicle or the dual-PEGylated human FGF21 variant (E37C, R77C, P171G) (1 mg/kg); measurements were obtained on Day 0 (72 hours post fifth STZ injection) and on Days 2, 4, 6, 8, 10, 12, 14, 16, 18, 20 and 22.

FIG. 17 is a plot showing plasma glucose levels measured in multiple low dose (MLD) streptozotocin-induced type 1 diabetic mice which were administered vehicle or the dual-PEGylated FGF21 variant (E37C, R77C, P171G) (1 mg/kg); blood glucose was measured prior to injection on Day-2, and on Days 2, 6, 10 and 14.

FIG. 18 is a bar graph showing insulin levels measured in MLD streptozotocin-induced type 1 diabetic mice which were administered vehicle or the dual-20 kd PEGylated FGF21 variant (E37C, R77C, P171G) (1 mg/kg) on Day-20 (post fifth STZ injection) and on Day 18 (two days post last injection of dual-PEGylated FGF21 variant (E37C, R77C, P171G) on Day 18).

FIG. 19 is a bar graph showing triglyceride levels measured in MLD streptozotocin-induced type 1 diabetic mice which were administered vehicle or the dual-20 kd PEGylated FGF21 variant (E37C, R77C, P171G) (1 mg/kg) on Day-20 (post fifth STZ injection) and on Day 18 (two days post last injection of dual-PEGylated FGF21 variant (E37C, R77C, P171G) on Day 18).

FIG. 20 is a bar graph showing cholesterol levels measured in MLD streptozotocin-induced type 1 diabetic mice which were administered vehicle or the dual-20 kd PEGylated FGF21 variant (E37C, R77C, P171G) (1 mg/kg) on Day-20 (post fifth STZ injection) and on Day 18 (two days post last injection of dual-PEGylated FGF21 variant (E37C, R77C, P171G) on Day 18).

FIG. 21 is a bar graph showing HDL levels measured in MLD streptozotocin-induced type 1 diabetic mice which were administered vehicle or the dual-20 kd PEGylated FGF21 variant (E37C, R77C, P171G) (1 mg/kg) on Day-20 (post fifth STZ injection) and on Day 18 (two days post last injection of dual-PEGylated FGF21 variant (E37C, R77C, P171G) on Day 18).

FIG. 22 is a bar graph showing NEFA levels measured in MLD streptozotocin-induced type 1 diabetic mice which were administered vehicle or the dual-20 kd PEGylated FGF21 variant (E37C, R77C, P171G) (1 mg/kg) on Day-20 (post fifth STZ injection) and on Day 18 (two days post last injection of dual-PEGylated FGF21 variant (E37C, R77C, P171G) on Day 18).

FIG. 23 is a bar graph showing insulin levels measured in MLD streptozotocin-induced type 1 diabetic mice which were administered vehicle or the dual-20 kd PEGylated FGF21 variant (E37C, R77C, P171G) (1 mg/kg) on Day-20 (post fifth STZ injection) and on Day 18 (two days post last injection of dual-PEGylated FGF21 variant (E37C, R77C, P171G) on Day 18).

FIG. 24 is a bar graph showing AST levels measured in MLD streptozotocin-induced type 1 diabetic mice which were administered vehicle or the dual-20 kd PEGylated FGF21 variant (E37C, R77C, P171G) (1 mg/kg) on Day-20 (post fifth STZ injection) and on Day 18 (two days post last injection of dual-PEGylated FGF21 variant (E37C, R77C, P171G) on Day 18).

FIG. 25 is a bar graph showing ALT levels measured in MLD streptozotocin-induced type 1 diabetic mice which were administered vehicle or the dual-PEGylated human FGF21 variant (E37C, R77C, P171G) (1 mg/kg) on Day-20 (post fifth STZ injection) and on Day 18 (two days post last injection of dual-PEGylated FGF21 variant (E37C, R77C, P171G) on Day 18).

FIG. 26 is a plot showing the change in body weight measured in MLD streptozotocin-induced type 1 diabetic mice, which were administered vehicle or the dual-PEGylated human FGF21 variant (E37C, R77C, P171G) (1 mg/kg); measurements were obtained on Day 0 (23 days post fifth STZ injection) and on Days 2, 4, 6, 8, 10, 12, 14, 16 and 18.

FIG. 27 is a photomicrograph showing insulin immunoreactivity in islets from streptozotocin-treated mice; upper panels are islets from vehicle-treated mice (A3) and lower panels from FGF21-treated mice (B3). Original magnification was ˜25×.

FIG. 28 is a table summarizing the insulin immunoreactivity and morphometric findings from each vehicle and PEG-FGF21 treated mouse; vehicle-treated mice are denoted A1 through A5, while PEG-FGF21 treated mice are denoted as B1 through B5.

### DETAILED DESCRIPTION OF THE INVENTION

The instant disclosure provides a method of treating Type 1 diabetes by administering to a subject in need thereof a therapeutically effective amount of an isolated human FGF21 polypeptide. Methods of administration and delivery are also provided.

Recombinant polypeptide and nucleic acid methods used herein, including in the Examples, are generally those set forth in Sambrook et al., Molecular Cloning: A Laboratory Manual (Cold Spring Harbor Laboratory Press, 1989) or Current Protocols in Molecular Biology (Ausubel et al., eds., Green Publishers Inc. and Wiley and Sons 1994), both of which are incorporated herein by reference for any purpose.

### I. General Definitions

Following convention, as used herein “a” and “an” mean “one or more” unless specifically indicated otherwise.

As used herein, the terms “amino acid” and “residue” are interchangeable and, when used in the context of a peptide or polypeptide, refer to both naturally occurring and synthetic amino acids, as well as amino acid analogs, amino acid mimetics and non-naturally occurring amino acids that are chemically similar to the naturally occurring amino acids.

A “naturally occurring amino acid” is an amino acid that is encoded by the genetic code, as well as those amino acids that are encoded by the genetic code that are modified after synthesis, e.g., hydroxyproline, γ-carboxyglutamate, and O-phosphoserine. An amino acid analog is a compound that has the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs can have modified R groups (e.g., norleucine) or modified peptide backbones, but will retain the same basic chemical structure as a naturally occurring amino acid.

An “amino acid mimetic” is a chemical compound that has a structure that is different from the general chemical structure of an amino acid, but that functions in a manner similar to a naturally occurring amino acid. Examples include a methacryloyl or acryloyl derivative of an amide, β-, γ-, δ-imino acids (such as piperidine-4-carboxylic acid) and the like.

A “non-naturally occurring amino acid” or a “non-naturally encoded amino acid,” which terms can be used interchangeably in the instant disclosure, is a compound that has the same basic chemical structure as a naturally occurring amino acid, but is not incorporated into a growing polypeptide chain by the in vivo translation complex. “Non-naturally occurring amino acid” also includes, but is not limited to, amino acids that occur by modification (e.g., posttranslational modifications) of a naturally encoded amino acid (including but not limited to, the 20 common amino acids) but are not themselves naturally incorporated into a growing polypeptide chain by the translation complex. A non-limiting lists of examples of non-naturally occurring amino acids that can be inserted into a polypeptide sequence or substituted for a wild-type residue in polypeptide sequence include β-amino acids, homoamino acids, cyclic amino acids and amino acids with derivatized side chains. Examples include (in the L-form or D-form; abbreviated as in parentheses): citrulline (Cit), homocitrulline (hCit), Nα-methylcitrulline (NMeCit), Nα-methylhomocitrulline (Nα-MeHoCit), ornithine (Orn), Nα-Methylornithine (Nα-MeOrn or NMeOrn), sarcosine (Sar), homolysine (hLys or hK), homoarginine (hArg or hR), homoglutamine (hQ), Nα-methylarginine (NMeR), Nα-methylleucine (Nα-MeL or NMeL), N-methylhomolysine (NMeHoK), Nα-methylglutamine (NMeQ), norleucine (Nle), norvaline (Nva), 1,2,3,4-tetrahydroisoquinoline (Tic), Octahydroindole-2-carboxylic acid (Oic), β-(1-naphthyl)alanine (1-Nal), β-(2-naphthyl)alanine (2-Nal), 1,2,3,4-tetrahydroisoquinoline (Tic), 2-indanylglycine (IgI), para-iodophenylalanine (pI-Phe), para-aminophenylalanine (4AmP or 4-Amino-Phe), 4-guanidino phenylalanine (Guf), glycyllysine (abbreviated “K(Nε-glycyl)” or “K(glycyl)” or “K(gly)”), nitrophenylalanine (nitrophe), aminophenylalanine (aminophe or Amino-Phe), benzylphenylalanine (benzylphe), γ-carboxyglutamic acid (γ-carboxyglu), hydroxyproline (hydroxypro), p-carboxyl-phenylalanine (Cpa), α-aminoadipic acid (Aad), Nα-methyl valine (NMeVal), N-α-methyl leucine (NMeLeu), Nα-methylnorleucine (NMeNle), cyclopentylglycine (Cpg), cyclohexylglycine (Chg), acetylarginine (acetylarg), β,β-diaminopropionoic acid (Dpr), α,γ-diaminobutyric acid (Dab), diaminopropionic acid (Dap), cyclohexylalanine (Cha), 4-methyl-phenylalanine (MePhe), β,β-diphenyl-alanine (BiPhA), aminobutyric acid (Abu), 4-phenyl-phenylalanine (or biphenylalanine; 4Bip), α-amino-isobutyric acid (Aib), beta-alanine, beta-aminopropionic acid, piperidinic acid, aminocaprioic acid, aminoheptanoic acid, aminopimelic acid, desmosine, diaminopimelic acid, N-ethylglycine, N-ethylaspargine, hydroxylysine, allo-hydroxylysine, isodesmosine, allo-isoleucine, N-methylglycine, N-methylisoleucine, N-methylvaline, 4-hydroxyproline (Hyp), γ-carboxyglutamate, ε-N,N,N-trimethyllysine, ε-N-acetyllysine, O-phosphoserine, N-acetylserine, N-formylmethionine, 3-methylhistidine, 5-hydroxylysine, ω-methylarginine, 4-Amino-O-Phthalic Acid (4APA), and other similar amino acids, and derivatized forms of any of those specifically listed.

The term “isolated nucleic acid molecule” refers to a single or double-stranded polymer of deoxyribonucleotide or ribonucleotide bases read from the 5′ to the 3′ end (e.g., a native or variant FGF21 nucleic acid sequence provided herein), or an analog thereof, that has been separated from at least about 50 percent of polypeptides, peptides, lipids, carbohydrates, polynucleotides or other materials with which the nucleic acid is naturally found when total nucleic acid is isolated from the source cells. Preferably, an isolated nucleic acid molecule is substantially free from any other contaminating nucleic acid molecules or other molecules that are found in the natural environment of the nucleic acid that would interfere with its use in polypeptide production or its therapeutic, diagnostic, prophylactic or research use.

The term “isolated polypeptide” refers to a polypeptide (e.g., a FGF21 polypeptide or variant FGF21 polypeptide provided herein) that has been separated from at least about 50 percent of polypeptides, peptides, lipids, carbohydrates, polynucleotides, or other materials with which the polypeptide is naturally found when isolated from a source cell. Preferably, the isolated polypeptide is substantially free from any other contaminating polypeptides or other contaminants that are found in its natural environment that would interfere with its therapeutic, diagnostic, prophylactic or research use.

The term “encoding” refers to a polynucleotide sequence encoding one or more amino acids. The term does not require a start or stop codon. An amino acid sequence can be encoded in any one of six different reading frames provided by a polynucleotide sequence.

The terms “identical” and percent “identity,” in the context of two or more nucleic acids or polypeptide sequences, refer to two or more sequences or subsequences that are the same. “Percent identity” means the percent of identical residues between the amino acids or nucleotides in the compared molecules and is calculated based on the size of the smallest of the molecules being compared. For these calculations, gaps in alignments (if any) can be addressed by a particular mathematical model or computer program (i.e., an “algorithm”). Methods that can be used to calculate the identity of the aligned nucleic acids or polypeptides include those described in Computational Molecular Biology, (Lesk, A. M., ed.), (1988) New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., (1987) Sequence Analysis in Molecular Biology, New York: Academic Press; Sequence Analysis Primer, (Gribskov, M. and Devereux, J., eds.), 1991, New York: M. Stockton Press; and Carillo et al., (1988) SIAM J. Applied Math. 48:1073.

In calculating percent identity, the sequences being compared are aligned in a way that gives the largest match between the sequences. The computer program used to determine percent identity is the GCG program package, which includes GAP (Devereux et al., (1984) Nucl. Acid Res. 12:387; Genetics Computer Group, University of Wisconsin, Madison, Wis.). The computer algorithm GAP is used to align the two polypeptides or polynucleotides for which the percent sequence identity is to be determined. The sequences are aligned for optimal matching of their respective amino acid or nucleotide (the “matched span”, as determined by the algorithm). A gap opening penalty (which is calculated as 3× the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. In certain embodiments, a standard comparison matrix (see, Dayhoff et al., (1978) Atlas of Protein Sequence and Structure 5:345-352 for the PAM 250 comparison matrix; Henikoff et al., (1992) Proc. Natl. Acad. Sci. U.S.A. 89:10915-10919 for the BLOSUM 62 comparison matrix) is also used by the algorithm.

Recommended parameters for determining percent identity for polypeptides or nucleotide sequences using the GAP program are the following:

Algorithm: Needleman et al., 1970, J. Mol. Biol. 48:443-453;

Comparison matrix: BLOSUM 62 from Henikoff et al., 1992, supra;

Gap Penalty: 12 (but with no penalty for end gaps)

Gap Length Penalty: 4

Threshold of Similarity: 0

Certain alignment schemes for aligning two amino acid sequences can result in matching of only a short region of the two sequences, and this small aligned region can have very high sequence identity even though there is no significant relationship between the two full-length sequences. Accordingly, the selected alignment method (e.g., the GAP program) can be adjusted if so desired to result in an alignment that spans at least 50 contiguous amino acids of the target polypeptide.

The terms “FGF21 polypeptide” and “FGF21 protein” are used interchangeably and mean a naturally-occurring wild-type polypeptide expressed in a mammal, such as a human or a mouse. For purposes of this disclosure, the term “FGF21 polypeptide” can be used interchangeably to refer to any full-length FGF21 polypeptide, e.g., SEQ ID NOs:2 and 4, which consist of 209 amino acid residues and which are encoded by the nucleotide sequence SEQ ID NOs:1 and 3; and any form comprising the mature form of the polypeptide, e.g., SEQ ID NOs:4 and 8, which consists of 181 amino acid residues and which are encoded by the nucleotide sequences SEQ ID NOs:3 and 5, and in which the 28 amino acid residues at the amino-terminal end of the full-length FGF21 polypeptide (i.e., which constitute the signal peptide) have been removed. FGF21 polypeptides can but need not comprise an amino-terminal methionine, which may be introduced by engineering or as a result of a bacterial expression process.

The term “FGF21 polypeptide” also encompasses a FGF21 polypeptide in which a naturally occurring FGF21 polypeptide sequence (e.g., SEQ ID NOs:2, 4, 6 and 8) has been modified, thus generating an “FGF21 variant.” Such modifications include, but are not limited to, one or more amino acid substitutions, including substitutions with non-naturally occurring amino acids non-naturally-occurring amino acid analogs and amino acid mimetics, and truncations. For example, it is known that human FGF21 retains activity when truncated on the N-terminus by 1, 2, 3, 4, 5, 6, 7, or 8 residues and on the C-terminus by 1, 2, 3, 4, 5, 6, 7, 8, 9, 10, 11, 12 or 13 residues (which presumably comprise receptor and β-Klotho binding sites, respectively; see, e.g., WO2009/149171). Accordingly, truncated variants of the 181 residue sequence of SEQ ID NOs:2 or 4 can be employed in the instant invention. The term “FGF21 polypeptide” encompasses point mutants that can be introduced into an FGF21 polypeptide, for example those shown in Tables 1-13. Moreover, it is known that human FGF21 exists in nature in at least two isoforms; one isoform comprises a Proline residue at position 174 of the full-length protein (SEQ ID NO:2) (position 146 of the mature form of the protein (SEQ ID NO:4)), while another comprises a Leucine residue at this position (shown in SEQ ID NOs:6 and 8, full-length and mature forms, respectively). Any of these isoforms can be employed in the disclosed compositions and methods and are encompassed by the terms “FGF21 polypeptide,” “FGF21 protein,” and “FGF21 variant.”

In various embodiments, a FGF21 polypeptide or FGF21 variant comprises an amino acid sequence that is at least about 85 percent identical to a naturally-occurring FGF21 polypeptide (e.g., SEQ ID NOs:2, 4, 6 and 8). In other embodiments, a FGF21 polypeptide comprises an amino acid sequence that is at least about 90 percent, or about 95, 96, 97, 98, or 99 percent identical to a naturally-occurring FGF21 polypeptide amino acid sequence (e.g., SEQ ID NOs:2, 4, 6 and 8). Such FGF21 polypeptides preferably, but need not, possess at least one activity of a wild-type FGF21 polypeptide, such as the ability to lower blood glucose, insulin, triglyceride, or cholesterol levels; the ability to reduce body weight; or the ability to improve glucose tolerance, energy expenditure, or insulin sensitivity. The present invention also encompasses nucleic acid molecules encoding such FGF21 polypeptide and FGF21 variant sequences.

As stated, a human FGF21 polypeptide or FGF21 variant can comprise a signal sequence (residues 1-28 of SEQ ID NOs:2 or 6) or it can have the signal sequence removed (providing the 181 residue sequence of SEQ ID NOs:4 or 8), which is the active form of FGF21 in vivo. In some instances, a FGF21 polypeptide or FGF21 variant can be used to treat or ameliorate a metabolic disorder in a subject is a mature form of FGF21 polypeptide or FGF21 variant that is derived from the same species as the subject.

A FGF21 polypeptide or FGF21 variant is preferably biologically active. In various respective embodiments, a FGF21 polypeptide or FGF21 variant has a biological activity that is equivalent to, greater to or less than that of the naturally occurring form of the mature FGF21 polypeptide or FGF21 variant from which the signal peptide has been removed from the N-terminus of the full length FGF21 polypeptide or FGF21 variant sequence. Examples of biological activities include the ability to lower blood glucose, insulin, triglyceride, or cholesterol levels; the ability to reduce body weight; or the ability to improve glucose tolerance, lipid tolerance, or insulin sensitivity; the ability to lower urine glucose and protein excretion.

The terms “therapeutically effective dose” and “therapeutically effective amount,” as used herein, means an amount of FGF21 polypeptide or FGF21 variant that elicits a biological or medicinal response in a tissue system, animal, or human being sought by a researcher, physician, or other clinician, which includes alleviation or amelioration of the symptoms of the disease or disorder being treated, i.e., an amount of a FGF21 polypeptide or FGF21 variant that supports an observable level of one or more desired biological or medicinal response, for example lowering blood glucose, insulin, triglyceride, or cholesterol levels; reducing body weight; or improving glucose tolerance, energy expenditure, or insulin sensitivity to a desired (e.g., physiologically normal for a human) level as determined using standard assays known to those of skill in the art. Examples of suitable assays to determine are provided herein and can be performed in an automated fashion using commercially-available instruments, such as an Olympus AU400e Chemistry Analyzer (Olympus America, Inc; Center Valley, Pa.) or a Human Multiplex Endocrine Kit (HENDO-75K, Millipore Corp., Billerica, Mass.).

II. FGF21 Polypeptides, FGF21 Variants and Nucleic Acids that can be Employed in the Disclosed Methods

The various methods provided herein can employ any FGF21 polypeptide or FGF21 variant described by the instant disclosure. These FGF21 polypeptides and FGF21 variants can be engineered and/or produced using standard molecular biology methodology. In various examples, a nucleic acid sequence encoding a FGF21 polypeptide or FGF21 variant, which can comprise all or a portion of SEQ ID NOs:1, 3, 5 and 7 can be isolated and/or amplified from genomic DNA, or cDNA using appropriate oligonucleotide primers. Primers can be designed based on the nucleic and amino acid sequences provided herein according to standard (RT)-PCR amplification techniques. The amplified FGF21 nucleic acid can then be cloned into a suitable vector and characterized by DNA sequence analysis.

Oligonucleotides for use as probes in isolating or amplifying all or a portion of the FGF21 polypeptides or FGF21 variants provided herein can be designed and generated using standard synthetic techniques, e.g., automated DNA synthesis apparatus, or can be isolated from a longer sequence of DNA.

### II.A. Naturally-Occurring and Variant FGF21 Polypeptide and Polynucleotide Sequences

In vivo, FGF21 is expressed as a contiguous amino acid sequence comprising a signal sequence.

The 209 amino acid sequence of full length human FGF21 (Pro 174/146 form) is:

(SEQ ID NO: 1)
MDSDETGFEHSGLWVSVLAGLLLGACQAHPIPDSSPLLQFGGQVRQR
YLYTDDAQQTEAHLEIREDGTVGGAADQSPESLLQLKALKPGVIQILG
VKTSRFLCQRPDGALYGSLHFDPEACSFRELLLEDGYNVYQSEAHGLP
LHLPGNKSPHRDPAPRGPARFLPLPGLPPAPPEPPGILAPQPPDVGSSDP
LSMVGPSQGRSPSYAS

and is encoded by the DNA sequence

(SEQ ID NO: 2)
atggactcggacgagaccgggttcgagcactcaggactgtgggtttctgtgctggctggtcttctgctgggagc
ctgccaggcacaccccatccctgactccagtcctctcctgcaattcgggggccaagtccggcagcggtacctct
acacagatgatgcccagcagacagaagcccacctggagatcagggaggatgggacggtggggggcgctgc
tgaccagagccccgaaagtctcctgcagctgaaagccttgaagccgggagttattcaaatcttgggagtcaaga
catccaggttcctgtgccagcggccagatggggccctgtatggatcgctccactttgaccctgaggcctgcagc
ttccgggagctgcttcttgaggacggatacaatgtttaccagtccgaagcccacggcctcccgctgcacctgcc
agggaacaagtccccacaccgggaccctgcaccccgaggaccagctcgcttcctgccactaccaggcctgcc
ccccgcacccccggagccacccggaatcctggccccccagccccccgatgtgggctcctcggaccctctga
gcatggtgggaccttcccagggccgaagccccagctacgcttcc.

The amino acid sequence of human FGF21 following cleavage of the 28 residue signal sequence is:

(SEQ ID NO: 3)
HPIPDSSPLLQFGGQVRQRYLYTDDAQQTEAHLEIREDGTVGGAADQS
PESLLQLKALKPGVIQILGVKTSRFLCQRPDGALYGSLHFDPEACSFRE
LLLEDGYNVYQSEAHGLPLHLPGNKSPHRDPAPRGPARFLPLPGLPPA
PPEPPGILAPQPPDVGSSDPLSMVGPSQGRSPSYAS

and is encoded by the DNA sequence

(SEQ ID NO: 4)
caccccatccctgactccagtcctctcctgcaattcgggggccaagtccggcagcggtacctctacacagatga
tgcccagcagacagaagcccacctggagatcagggaggatgggacggtggggggcgctgctgaccagagc
cccgaaagtctcctgcagctgaaagccttgaagccgggagttattcaaatcttgggagtcaagacatccaggttc
ctgtgccagcggccagatggggccctgtatggatcgctccactttgaccctgaggcctgcagcttccgggagct
gcttcttgaggacggatacaatgtttaccagtccgaagcccacggcctcccgctgcacctgccagggaacaagt
ccccacaccgggaccctgcaccccgaggaccagctcgcttcctgccactaccaggcctgccccccgcacccc
cggagccacccggaatcctggccccccagccccccgatgtgggctcctcggaccctctgagcatggtgggac
cttcccagggccgaagccccagctacgcttcc.

As has been stated herein, human FGF 21 can also exist in a naturally-occurring isoform in which the Proline at position 174 of SEQ ID NO:2 (position 146 in SEQ ID NO:4) is replaced with a Leucine. The amino acid and nucleic acid sequences associated with this form of FGF21 are provided herein as SEQ ID NOs:5-8.

As stated herein, the term “FGF21 polypeptide” refers to a FGF21 polypeptide comprising the human amino acid sequences SEQ ID NOs:2, 4, 6 and 8. The term “FGF21 polypeptide,” however, also encompasses polypeptides comprising an amino acid sequence that differs from the amino acid sequence of a naturally occurring FGF21 polypeptide sequence, e.g., SEQ ID NOs:2, 4, 6 and 8, by one or more amino acids such that the sequence is at least 85% identical to SEQ ID NOs:2, 4, 6 and 8; such polypeptides are generally referred to in the instant disclosure as “FGF21 variants” and are described further herein. FGF21 polypeptides can be generated by introducing one or more amino acid substitutions, either conservative or non-conservative and using naturally or non-naturally occurring amino acids, at particular positions of the FGF21 polypeptide. Examples of substitutions that can be introduced into a FGF21 polypeptide are shown in Tables 1-13 and described herein.

A “conservative amino acid substitution” can involve a substitution of a native amino acid residue (i.e., a residue found in a given position of the wild-type FGF21 polypeptide sequence) with a normative residue (i.e., a residue that is not found in a given position of the wild-type FGF21 polypeptide sequence) such that there is little or no effect on the polarity or charge of the amino acid residue at that position. Conservative amino acid substitutions also encompass non-naturally occurring amino acid residues that are typically incorporated by chemical peptide synthesis rather than by synthesis in biological systems. These include peptidomimetics, and other reversed or inverted forms of amino acid moieties.

Naturally occurring residues can be divided into classes based on common side chain properties, as shown in Table 1:

TABLE 1
Conservative Substitutions

| | |
|---|---|
| hydrophobic | norleucine, Met, Ala, Val, Leu, Ile |
| neutral hydrophilic | Cys, Ser, Thr |
| acidic | Asp, Glu |
| basic | Asn, Gln, His, Lys, Arg |
| residues that influence chain | Gly, Pro |
| orientation | |
| aromatic | Trp, Tyr, Phe |

Additional groups of amino acids can also be formulated using the principles described in, e.g., Creighton (1984) PROTEINS: STRUCTURE AND MOLECULAR PROPERTIES (2d Ed. 1993), W.H. Freeman and Company. In some instances it can be useful to further characterize substitutions based on two or more of such features (e.g., substitution with a “small polar” residue, such as a Thr residue, can represent a highly conservative substitution in an appropriate context).

Conservative substitutions can involve the exchange of a member of one of these classes for another member of the same class. Non-conservative substitutions can involve the exchange of a member of one of these classes for a member from another class.

Synthetic, rare, or modified amino acid residues having known similar physiochemical properties to those of an above-described grouping can be used as a “conservative” substitute for a particular amino acid residue in a sequence. For example, a D-Arg residue may serve as a substitute for a typical L-Arg residue. It also can be the case that a particular substitution can be described in terms of two or more of the above described classes (e.g., a substitution with a small and hydrophobic residue means substituting one amino acid with a residue(s) that is found in both of the above-described classes or other synthetic, rare, or modified residues that are known in the art to have similar physiochemical properties to such residues meeting both definitions).

Nucleic acid sequences encoding a FGF21 polypeptide provided herein, including those degenerate to SEQ ID NOs:1, 3, 5 and 7, and those encoding polypeptide variants of SEQ ID NOs:1, 3, 5 and 7 such as those comprising the mutations of Tables 1-13, form other aspects of the instant disclosure.

### II.B. FGF21 Vectors

In order to express the FGF21 nucleic acid sequences provided herein, thereby generating a FGF21 polypeptide or FGF21 variant for use in the disclosed methods, the appropriate coding sequences, e.g., SEQ ID NOs:1, 3, 5 and 7 or a sequence encoding one or more mutants of Tables 1-13, can be cloned into a suitable vector and, after introduction in a suitable host, the sequence can be expressed to produce the encoded polypeptide according to standard cloning and expression techniques, (as described in, e.g., Sambrook et al., Molecular Cloning: A Laboratory Manual (Cold Spring Harbor Laboratory Press, 1989). The instant disclosure also relates to such vectors comprising a nucleic acid sequence provided herein (e.g., a sequence encoding a FGF21 polypeptide or FGF21 variant).

A “vector” refers to a delivery vehicle that (a) promotes the expression of a polypeptide-encoding nucleic acid sequence; (b) promotes the production of the polypeptide therefrom; (c) promotes the transfection/transformation of target cells therewith; (d) promotes the replication of the nucleic acid sequence; (e) promotes stability of the nucleic acid; (f) promotes detection of the nucleic acid and/or transformed/transfected cells; and/or (g) otherwise imparts advantageous biological and/or physiochemical function to the polypeptide-encoding nucleic acid. A vector can be any suitable vector, including chromosomal, non-chromosomal, and synthetic nucleic acid vectors (a nucleic acid sequence comprising a suitable set of expression control elements). Examples of such vectors include derivatives of SV40, bacterial plasmids, phage DNA, baculovirus, yeast plasmids, vectors derived from combinations of plasmids and phage DNA, and viral nucleic acid (RNA or DNA) vectors.

A recombinant expression vector can be designed for expression of a FGF21 protein in prokaryotic (e.g., E. coli) or eukaryotic cells (e.g., insect cells, using baculovirus expression vectors, yeast cells, or mammalian cells). Representative host cells include those hosts typically used for cloning and expression, including Escherichia coli strains TOP10F′, TOP10, DH10B, DH5a, HB101, W3110, BL21(DE3) and BL21 (DE3)pLysS, BLUESCRIPT (Stratagene), mammalian cell lines CHO, CHO-K1, HEK293, 293-EBNA pIN vectors (Van Heeke & Schuster, J. Biol. Chem. 264: 5503-5509 (1989)); pET vectors (Novagen, Madison Wis.). Alternatively, the recombinant expression vector can be transcribed and translated in vitro, for example using T7 promoter regulatory sequences and T7 polymerase and an in vitro translation system. Preferably, the vector contains a promoter upstream of the cloning site containing the nucleic acid sequence encoding the polypeptide. Examples of promoters, which can be switched on and off, include the lac promoter, the T7 promoter, the trc promoter, the tac promoter and the trp promoter.

Thus, provided herein are vectors comprising a nucleic acid sequence encoding a FGF21 polypeptide or a FGF21 variant, that facilitate the expression of recombinant FGF21 polypeptides that can be employed in the disclosed methods. In various embodiments, the vectors comprise an operably linked nucleotide sequence which regulates the expression of a FGF21 polypeptide or variant. A vector can comprise or be associated with any suitable promoter, enhancer, and other expression-facilitating elements. Examples of such elements include strong expression promoters (e.g., a human CMV IE promoter/enhancer, an RSV promoter, SV40 promoter, SL3-3 promoter, MMTV promoter, or HIV LTR promoter, EF1alpha promoter, CAG promoter), effective poly (A) termination sequences, an origin of replication for plasmid product in E. coli, an antibiotic resistance gene as a selectable marker, and/or a convenient cloning site (e.g., a polylinker). Vectors also can comprise an inducible promoter as opposed to a constitutive promoter such as CMV IE. In one aspect, a nucleic acid comprising a sequence encoding a FGF21 polypeptide or FGF21 variant which is operatively linked to a tissue specific promoter which promotes expression of the sequence in a metabolically-relevant tissue, such as liver or pancreatic tissue is provided.

### II.C. Host Cells

In another aspect of the instant disclosure, host cells comprising the FGF21 nucleic acids and vectors disclosed herein are provided. In various embodiments, the vector or nucleic acid is integrated into the host cell genome, which in other embodiments the vector or nucleic acid is extra-chromosomal.

Recombinant cells, such as yeast, bacterial (e.g., E. coli), and mammalian cells (e.g., immortalized mammalian cells) comprising such a nucleic acid, vector, or combinations of either or both thereof are provided. In various embodiments cells comprising a non-integrated nucleic acid, such as a plasmid, cosmid, phagemid, or linear expression element, which comprises a sequence coding for expression of a FGF21 polypeptide or variant for use in the disclosed methods, are provided.

A vector comprising a nucleic acid sequence encoding a FGF21 polypeptide or variant provided herein can be introduced into a host cell by transformation or by transfection. Methods of transforming a cell with an expression vector are well known.

A FGF21 polypeptide or FGF21 variant-encoding nucleic acid can be positioned in and/or delivered to a host cell or host animal via a viral vector. Any suitable viral vector can be used in this capacity. A viral vector can comprise any number of viral polynucleotides, alone or in combination with one or more viral proteins, which facilitate delivery, replication, and/or expression of the nucleic acid of the invention in a desired host cell. The viral vector can be a polynucleotide comprising all or part of a viral genome, a viral protein/nucleic acid conjugate, a virus-like particle (VLP), or an intact virus particle comprising viral nucleic acids and a FGF21 polypeptide or variant-encoding nucleic acid. A viral particle viral vector can comprise a wild-type viral particle or a modified viral particle. The viral vector can be a vector which requires the presence of another vector or wild-type virus for replication and/or expression (e.g., a viral vector can be a helper-dependent virus), such as an adenoviral vector amplicon. Typically, such viral vectors consist of a wild-type viral particle, or a viral particle modified in its protein and/or nucleic acid content to increase transgene capacity or aid in transfection and/or expression of the nucleic acid (examples of such vectors include the herpes virus/AAV amplicons). Typically, a viral vector is similar to and/or derived from a virus that normally infects humans. Suitable viral vector particles in this respect, include, for example, adenoviral vector particles (including any virus of or derived from a virus of the adenoviridae), adeno-associated viral vector particles (AAV vector particles) or other parvoviruses and parvoviral vector particles, papillomaviral vector particles, flaviviral vectors, alphaviral vectors, herpes viral vectors, pox virus vectors, retroviral vectors, including lentiviral vectors.

### II.D. Isolation of a FGF21 Polypeptide or FGF21 Variant

A FGF21 polypeptide or FGF21 variant expressed as described herein can be isolated using standard protein purification methods. A FGF21 polypeptide or variant can be isolated from a cell in which is it naturally expressed or it can be isolated from a cell that has been engineered to express a FGF21 polypeptide or FGF21 variant, for example a cell that does not naturally express any form of FGF21 polypeptide.

Protein purification methods that can be employed to isolate a FGF21 polypeptide or variant, as well as associated materials and reagents, are known in the art. Exemplary methods of purifying a FGF21 polypeptide are provided in the Examples presented herein and in WO2009/149171 and WO2010/129503.

### III. Specific FGF21 Variants

As stated herein, the term “FGF21 polypeptide” encompasses various mutant forms of human FGF21. The disclosed mutations can impart a variety of properties to an FGF21 polypeptide. For example, some of the disclosed mutations can enhance the half-life of a FGF21 polypeptide, and thereby enhance its therapeutic properties. Such enhancements can be desirable when performing the disclosed methods.

In one embodiment, it has been determined that the A180E mutation minimizes C-terminal degradation of mature human FGF21 (SEQ ID NO:4 or 8). Accordingly, the A180E mutation can form an element of a variant FGF21 sequence either as a single mutation or in combination with other mutations, as disclosed herein.

In another embodiment, it has been determined that the L98R mutation minimizes aggregation and enhances solubility of mature human FGF21 (SEQ ID NO:4 or 8). Accordingly, the L98R mutation can form an element of a variant FGF21 sequence either as a single mutation or in combination with other mutations, as disclosed herein.

In another embodiment, it has been determined that the P171G mutation minimizes proteolytic cleavage of mature human FGF21 (SEQ ID NO:4 or 8). Accordingly, the P171G mutation can form an element of a variant FGF21 sequence either as a single mutation or in combination with other mutations, as disclosed herein.

The mutations disclosed herein can impart various properties to an FGF21 polypeptide comprising SEQ ID NO:4 or 8; for example some of the disclosed mutations can enhance the stability of FGF21 by providing sites for the formation of disulfide bonds, thus providing enhanced proteolytic stability, for example when FGF21 is disposed in a formulation. Yet other disclosed mutations can provide increased or decreased levels of O-glycosylation when FGF21 is expressed in yeast. Still other mutations can disrupt points at which proteases or other chemical attacks may act on FGF21 to degrade it, including the C-terminus of FGF21. Other mutations can impart decreased deamidation. And still other mutations can reduce the levels of aggregation of FGF21 and consequently enhance its solubility. Mutations can also be introduced in order to serve as attachment points for half-life extending moieties, such as human serum albumin, polyethylene glycol (PEG) or an IgG constant region, as described herein. In various ways, these mutations can improve the in vivo or in vitro activity of FGF21 over native FGF21. As described herein, one or more mutations imparting one or more desired properties can be introduced into an FGF21 sequence to provide a cumulative enhancement of desirable properties, including properties that provide an enhanced therapeutic profile of a FGF21 polypeptide or FGF21 variant. Such enhancements can make a given FGF21 polypeptide or FGF21 variant more preferred for use in the disclosed methods.

In one example, single or pairs of cysteine residues can be introduced at various points in a mature human FGF21 sequence (SEQ ID NO:4 or 8) to facilitate the formation of disulfide bond formation. Introduced cysteine residues can also serve as sites for PEGylation. The naturally occurring disulfide bond between C75 and C93 can be maintained intact, or disrupted and a new disulfide bond formed between C75 or C93 and an introduced cysteine residue. Examples of positions at which a cysteine can be substituted for a wild-type residue are summarized in Table 2:

TABLE 2
Cysteine Mutations

| Position | Wild type | Introduced Mutation |
|---|---|---|
| 18 | Q | C |
| 19 | R | C |
| 20 | Y | C |
| 21 | L | C |
| 22 | Y | C |
| 23 | T | C |
| 24 | D | C |
| 25 | D | C |
| 26 | A | C |
| 27 | Q | C |
| 28 | Q | C |
| 29 | T | C |
| 30 | E | C |
| 31 | A | C |
| 33 | L | C |
| 35 | I | C |
| 36 | R | C |
| 37 | E | C |
| 38 | D | C |
| 39 | G | C |
| 40 | T | C |
| 41 | V | C |
| 42 | G | C |
| 43 | G | C |
| 44 | A | C |
| 45 | A | C |
| 46 | D | C |
| 47 | Q | C |
| 48 | S | C |
| 49 | P | C |
| 50 | E | C |
| 54 | Q | C |
| 56 | K | C |
| 57 | A | C |
| 58 | L | C |
| 59 | K | C |
| 60 | P | C |
| 61 | G | C |
| 62 | V | C |
| 64 | Q | C |
| 65 | I | C |
| 66 | L | C |
| 67 | G | C |
| 68 | V | C |
| 69 | K | C |
| 70 | T | C |
| 71 | S | C |
| 72 | R | C |
| 73 | F | C |
| 75 | C | C |
| 76 | Q | C |
| 77 | R | C |
| 78 | P | C |
| 79 | D | C |
| 80 | G | C |
| 81 | A | C |
| 82 | L | C |
| 83 | Y | C |
| 84 | G | C |
| 85 | S | C |
| 86 | L | C |
| 87 | H | C |
| 88 | F | C |
| 89 | D | C |
| 90 | P | C |
| 91 | E | C |
| 92 | A | C |
| 93 | C | C |
| 94 | S | C |
| 95 | F | C |
| 96 | R | C |
| 98 | L | C |
| 99 | L | C |
| 100 | L | C |
| 101 | E | C |
| 102 | D | C |
| 103 | G | C |
| 104 | Y | C |
| 106 | V | C |
| 107 | Y | C |
| 108 | Q | C |
| 109 | S | C |
| 110 | E | C |
| 111 | A | C |
| 112 | H | C |
| 113 | G | C |
| 114 | L | C |
| 115 | P | C |
| 116 | L | C |
| 117 | H | C |
| 118 | L | C |
| 119 | P | C |
| 120 | G | C |
| 121 | N | C |
| 122 | K | C |
| 123 | S | C |
| 124 | P | C |
| 125 | H | C |
| 126 | R | C |
| 127 | D | C |
| 128 | P | C |
| 129 | A | C |
| 130 | P | C |
| 131 | R | C |
| 132 | G | C |
| 133 | P | C |
| 134 | A | C |
| 135 | R | C |
| 137 | L | C |
| 138 | P | C |
| 139 | L | C |
| 140 | P | C |
| 152 | I | C |
| 153 | L | C |
| 154 | A | C |
| 163 | S | C |
| 167 | S | C |

Introduced cysteine residues can facilitate the formation of engineered disulfide bonds. Such disulfide bonds can enhance the stability of an FGF21 polypeptide or FGF21 variant, including the stability of the molecule under concentrated conditions, such as in a therapeutic formulation. Examples of engineered disulfide bond pairs include those shown in Table 3 (positions refer to the mature human FGF21 polypeptide of SEQ ID NO:4 or 8):

TABLE 3
Engineered Disulfide Bonds

| Position | | Disulfide Bond Formed with |
|---|---|---|
| of | | a Naturally-Occurring or |
| Introduced | Wild type | Introduced Cysteine at |
| Cysteine | Residue | Position |
| 19 | R | 138 |
| 20 | Y | 139 |
| 21 | L | 33 |
| 22 | Y | 137, 139 |
| 23 | T | 25, 28 |
| 24 | D | 135 |
| 25 | D | 23, 122 |
| 26 | A | 122 |
| 27 | Q | 123 |
| 28 | Q | 28, 43, 124 |
| 31 | A | 43 |
| 33 | L | 21 |
| 35 | I | 84 |
| 41 | V | 82 |
| 42 | G | 124, 126 |
| 43 | G | 28, 31, 124 |
| 50 | E | 69 |
| 54 | Q | 66 |
| 58 | L | 62 |
| 62 | V | 58 |
| 66 | L | 54 |
| 67 | G | 72, 135 |
| 69 | K | 50 |
| 72 | R | 67, 84 |
| 73 | F | 93 |
| 75 | C | 85, 92 |
| 76 | Q | 109 |
| 77 | R | 79, 81 |
| 79 | D | 77 |
| 80 | G | 129 |
| 81 | A | 77 |
| 82 | L | 41, 119 |
| 84 | G | 35, 72 |
| 85 | S | 75 |
| 90 | P | 92 |
| 92 | A | 90 |
| 93 | C | 73 |
| 94 | S | 110 |
| 95 | F | 107 |
| 100 | L | 102 |
| 102 | D | 100, 104 |
| 104 | Y | 102 |
| 107 | Y | 95 |
| 109 | S | 76 |
| 110 | E | 94 |
| 115 | P | 117 |
| 117 | H | 115, 129, 130 |
| 118 | L | 132, 134 |
| 119 | P | 82 |
| 121 | N | 127 |
| 122 | K | 25, 26 |
| 123 | S | 27, 125 |
| 124 | P | 28, 42, 43 |
| 125 | H | 123 |
| 126 | R | 42 |
| 127 | D | 121, 132 |
| 129 | A | 80, 117 |
| 130 | P | 117 |
| 132 | G | 118, 127 |
| 134 | A | 118 |
| 135 | R | 24, 67 |
| 137 | L | 22 |
| 138 | P | 19 |
| 139 | L | 20, 22 |
| 152 | I | 163 |
| 163 | S | 152 |

The selection of one or more pairs of residues for mutation to cysteine residues with the goal of engineering a disulfide bond that is not found in wild-type FGF21 can be based on an analysis of a three-dimensional model of FGF21. For example, a rational protein engineering approach can be used to identify suitable residues in FGF21 for mutation. This can be achieved by inspection of a high resolution (1.3 Å) X-ray crystal structure of FGF19 obtained from the Protein Databank (“PDB”; e.g., structure 1PWA), which can then be used to create a 3D homology model of FGF21 using, e.g., the MOE (Molecular Operating Environment; Chemical Computing Group; Montreal, Quebec, Canada) modeling software. FGF19 is a useful template, since among the proteins deposited in the PDB, FGF19 is closely related protein to FGF21 in terms of amino acid sequence homology.

In another aspect, additional mutations can be introduced into a mature FGF21 sequence in order to enhance the stability of FGF21 under conditions of highly concentrated solutions or common formulation components such as phenol, m-cresol, methylparaben, resorcinol and benzyl alcohol. Examples of mutations that can provide the property of enhanced stability include those shown in Table 4 (positions refer to the mature human FGF21 polypeptide of SEQ ID NO:4 or 8):

TABLE 4
Stability-Enhancing Mutations

| Position | Wild type | Introduced Mutations |
|---|---|---|
| 42 | G | D, E, R, K, H, S, T, N, Q |
| 54 | Q | D, E, R, K, H, S, T, N, Q |
| 77 | R | D, E, R, K, H, S, T, N, Q |
| 81 | A | D, E, R, K, H, S, T, N, Q |
| 86 | L | D, E, R, K, H, S, T, N, Q |
| 88 | F | D, E, R, K, H, S, T, N, Q |
| 122 | K | D, E, R, K, H, S, T, N, Q |
| 125 | H | D, E, R, K, H, S, T, N, Q |
| 126 | R | D, E, R, K, H, S, T, N, Q |
| 130 | P | D, E, R, K, H, S, T, N, Q |
| 131 | R | D, E, R, K, H, S, T, N, Q |
| 139 | L | D, E, R, K, H, S, T, N, Q |
| 145 | A | D, E, R, K, H, S, T, N, Q |
| 146 | P | D, E, R, K, H, S, T, N, Q |
| 152 | I | D, E, R, K, H, S, T, N, Q |
| 154 | A | D, E, R, K, H, S, T, N, Q |
| 156 | Q | D, E, R, K, H, S, T, N, Q |
| 161 | G | D, E, R, K, H, S, T, N, Q |
| 163 | S | D, E, R, K, H, S, T, N, Q |
| 170 | G | D, E, R, K, H, S, T, N, Q |
| 172 | S | D, E, R, K, H, S, T, N, Q |

See, e.g., WO 2009/149171 and WO2010/129503, incorporated herein by reference.

The selection of one or more pairs of residues for mutation to a stability-enhancing mutation can be based on an analysis of a three-dimensional model of FGF21. For example, a rational protein engineering approach can be used to identify suitable residues in FGF21 for mutation. This can be achieved by inspection of a high resolution (1.3 Å) X-ray crystal structure of FGF19 (1PWA) obtained from the Protein Databank (PDB), which can then be used to create a 3D homology model of FGF21 using, e.g., the MOE (Molecular Operating Environment; Chemical Computing Group; Montreal, Quebec, Canada) modeling software. FGF19 is a useful template, since among the proteins deposited in the PDB, FGF19 is related protein to FGF21 in terms of the amino acid sequence homology.

In another aspect, additional mutations can be introduced into the FGF21 sequence in order to reduce the degree of proteolytic cleavage of a FGF21 polypeptide under some conditions. Examples of mutations that can provide the property of resistance to proteolytic cleavage include those shown in Table 5 (positions refer to the mature human FGF21 polypeptide of SEQ ID NO:4 or 8):

TABLE 5
Proteolysis-resistance Mutations

| Position | Wild Type | Indroduced Mutations |
|---|---|---|
| 19 | R | Q, I, K |
| 20 | Y | H, L, F |
| 21 | L | I, F, Y, V |
| 22 | Y | I, F, V |
| 150 | P | A, R |
| 151 | G | A, V |
| 152 | I | H, L, F, V |
| 170 | G | A, N, D, C, Q, E, P, S |
| 171 | P | A, R, N, D, C, E, Q, H, K, S, T, W, Y |
| 172 | S | L, T |
| 173 | Q | R, E |

See, e.g., WO 2009/149171 and WO2010/129503, incorporated herein by reference.

In a further aspect, additional mutations can be introduced into a mature FGF21 sequence in order to inhibit aggregation of a FGF21 polypeptide under some conditions, such as high concentration. Examples of mutations that can provide the property of inhibiting aggregation of FGF21 include those shown in Table 6 (positions refer to the mature human FGF21 polypeptide of SEQ ID NO:4 or 8):

TABLE 6
Aggregation-reducing Mutations

| Position | Wild-Type | Mutation |
|---|---|---|
| 26 | A | E, K, R |
| 45 | A | E, K, R, Q, T |
| 52 | L | T |
| 58 | L | C, E, S |
| 60 | P | A, E, K, R |
| 78 | P | A, C, H, R |
| 86 | L | C T |
| 88 | F | A, E, K, R, S |
| 98 | L | C, E, K, Q |
| 99 | L | C, D, E, R |
| 111 | A | K, T |
| 129 | A | D, E, H, K, N, R, Q |
| 134 | A | E, H, K, Y |

See, e.g., WO 2009/149171 and WO2010/129503, incorporated herein by reference.

In another embodiment, the present invention is directed to FGF21 variant polypeptides comprising one or more non-naturally occurring polymer attachment sites which have been capped by the addition of another one or more residues to the C-terminus of the polypeptide, extending the amino acid sequence beyond that of the wild-type protein. In yet another embodiment, the present disclosure is directed to FGF21 variant polypeptides comprising one or more non-naturally occurring polymer attachments sites that further comprise one or more C-terminal mutations. Such capped and C-terminally mutated FGF21 mutant polypeptides can, but need not, be chemically modified.

As used herein, the term “capped FGF21 variant polypeptide” refers to an FGF21 polypeptide or FGF21 variant, or to a chemically modified FGF21 polypeptide or FGF21 variant polypeptide in which one or more amino acid residues have been added to the C terminus of the FGF21 variant polypeptide or chemically modified FGF21 variant polypeptide. Any naturally or non-naturally occurring amino acid can be used to cap an FGF21 mutant polypeptide, including one or more proline residues and one or more glycine residues. Although the wild-type mature FGF21 sequence is 181 residues long (SEQ ID NO:4 or 8), a capped FGF21 polypeptide or FGF21 variant extends the length of the polypeptide one residue for each added capping residue; consistent with the numbering scheme of the present disclosure, cap residues are numbered beginning with 182. Thus, a single proline capping residue is indicated as P182. Longer caps are possible and are numbered accordingly (e.g., X182, Y183, Z184, where X, Y and Z are any naturally or non-naturally occurring amino acid). Capping residues can be added to a mutant FGF21 polypeptide using any convenient method, such as chemically, in which an amino acid is covalently attached to the C-terminus of the polypeptide by a chemical reaction. Alternatively, a codon encoding a capping residue can be added to the FGF21 mutant polypeptide coding sequence using standard molecular biology techniques. Any of the mutant FGF21 polypeptides described herein can be capped with one or more residues, as desired.

C-terminal mutations form another aspect of the present invention. As used herein, the term “C-terminal mutation” refers to one or more changes in the region of residues 91-181 (or longer if the polypeptide is capped) of a FGF21 polypeptide or FGF21 variant. A C-terminal mutation introduced into a FGF21 polypeptide or FGF21 variant sequence will be in addition to one or more mutations which introduce a non-naturally occurring polymer attachment site. Although C-terminal mutations can be introduced at any point in the region of 91-181 of the FGF21 polypeptide or FGF21 variant sequence, exemplary positions for C-terminal mutations include positions 171, 172, 173, 174, 175, 176, 177, 178, 179, 180 and 181. C-terminal mutations can be introduced using standard molecular biological techniques, such as those described herein. Any of the FGF21 polypeptides or FGF21 variants described herein can comprise a C-terminal mutation.

Examples of positions and identities for capped and/or C-terminally mutations are shown in Table 7:

TABLE 7
Examples of Capping Positions and/or C-terminally Mutations

E37C, R77C, P171G, P182
P171G, S181P, P182
P171G, S181P
P171G, S181T
P171G, S181G
P171G, S181A
P171G, S181L
P171G, A180P
P171G, A180G
P171G, A180S
P171G, Y179P
P171G, Y179G
P171G, Y179S
P171G, Y179A
P171G, L182
P171G, G182
P171G, P182
P171G, G182, G183
P171G, G182, G183, G184, G185, G186

The activity of capped and/or C-terminally FGF21 polypeptides and FGF21 variants, as well as chemically modified forms of these mutants, can be assayed in a variety of ways, for example, using an in vitro ELK-luciferase assay.

The activity of the capped and/or C-terminally mutated FGF21 polypeptides and FGF21 variants, and chemically modified capped and/or C-terminally mutated FGF21 polypeptides and FGF21 variants, of the present invention can also be assessed in an in vivo assay, such as an ob/ob mouse. Generally, to assess the in vivo activity of one or more of these polypeptides, the polypeptide can be administered to a test animal intraperitoneally. After one or more desired time periods, a blood sample can be drawn, and blood glucose levels can be measured.

As with all FGF21 polypeptides and FGF21 variants of the present invention, capped and/or C-terminally mutated FGF21 polypeptides and FGF21 variants, and chemically modified capped and/or C terminally mutated FGF21 polypeptides and FGF21 variants, can optionally comprise an amino-terminal methionine residue, which can be introduced by directed mutation or as a result of a bacterial expression process.

The capped and/or C-terminally mutated FGF21 polypeptides and FGF21 variants of the present invention can be prepared using standard laboratory techniques. Those of ordinary skill in the art, familiar with standard molecular biology techniques, can employ that knowledge, coupled with the instant disclosure, to make and use the capped and/or C-terminally mutated FGF21 polypeptides and FGF21 variants of the present invention. Standard techniques can be used for recombinant DNA, oligonucleotide synthesis, tissue culture, and transformation (e.g., electroporation, lipofection). See, e.g., Sambrook et al., Molecular Cloning: A Laboratory Manual, which is incorporated herein by reference for any purpose. Enzymatic reactions and purification techniques can be performed according to manufacturer's specifications, as commonly accomplished in the art, or as described herein. Unless specific definitions are provided, the nomenclatures utilized in connection with, and the laboratory procedures and techniques of, analytical chemistry, synthetic organic chemistry, and medicinal and pharmaceutical chemistry described herein are those well known and commonly used in the art. Standard techniques can be used for chemical syntheses; chemical analyses; pharmaceutical preparation, formulation, and delivery; and treatment of patients.

Following the preparation of a capped and/or C terminally mutated FGF21 mutant polypeptide, the polypeptide can be chemically modified by the attachment of a polymer, as described herein. See, e.g., WO2010/042747, incorporated herein by reference.

In a further aspect of the present invention, FGF21 polypeptides and FGF21 variants can be prepared in which both cysteine residues in a wild-type FGF21 polypeptide sequence (SEQ ID NO:4 or 8) are replaced with residues that do not form disulfide bonds and do not serve as polymer attachment sites, such as alanine or serine. Subsequently, substitutions can be made in the FGF21 mutant polypeptide sequence that introduce non-naturally occurring polymer attachment sites, in the form of thiol-containing residues (e.g., cysteine residues or non-naturally occurring amino acids having thiol groups) or free amino groups (e.g., lysine or arginine residues or non-naturally occurring amino acids having free amino groups). Polymers that rely on thiol or free amino groups for attachment, such as PEG, can then be targeted to cysteine, lysine or arginine residues that have been introduced into the FGF21 mutant polypeptide sequence at known positions. This strategy can facilitate more efficient and controlled polymer placement.

In one approach, the two naturally occurring cysteine residues in the wild-type FGF21 polypeptide, which are located at positions 75 and 93, can be substituted with non-thiol containing residues. Subsequently, a cysteine residue can be introduced at a known location. The FGF21 mutant polypeptide can also comprise other mutations, which can introduce still more polymer attachments sites (e.g., cysteine residues) or can be designed to achieve some other desired property. Examples of such FGF21 mutant polypeptides include C75A/E91C/C93A/H125C/P171G and C75S/E91C/C93S/H125C/P171G. In these examples, the naturally occurring cysteines at positions 75 and 93 have been mutated to alanine or serine residues, polymer attachment sites have been introduced at positions 91 and 125 (in this case for a thiol-reactive polymer such as PEG) and an additional mutation has been made at position 171, namely the substitution of proline 171 with a glycine residue (recited positions are relative to SEQ ID NO:4 or 8).

Like all of the FGF21 polypeptides and FGF21 variants disclosed herein, the activity of polypeptides which contain neither of the cysteines found in the wild-type mature FGF21 polypeptide sequence but instead comprise an introduced polymer attachment site and optionally one or more additional mutations, as well as chemically modified forms of these mutants, can be assayed in a variety of ways, for example, using an in vitro ELK-luciferase assay (see, e.g., WO2010/042747, which discloses an in vitro assay suitable for assaying the activity of any of the disclosed FGF21 polypeptides and FGF21 variants disclosed herein). The in vivo activity of these polypeptides can be assessed in an in vivo assay, such as using ob/ob mice (again, see, e.g., WO2010/042747, which discloses a in vivo assay suitable for assaying the activity of any of the disclosed FGF21 polypeptides and FGF21 variants disclosed herein).

As with all of the FGF21 polypeptides and FGF21 variants of the present invention, the activity of FGF21 variant polypeptides which contain neither of the cysteines found in the wild-type mature FGF21 polypeptide sequence but instead comprise an introduced polymer attachment site and optionally one or more additional mutations and chemically modified forms of these FGF21 variant polypeptides can optionally comprise an amino-terminal methionine residue, which can be introduced by directed mutation or as a result of a bacterial expression process.

FGF21 variants which contain neither of the cysteines found in the wild-type FGF21 polypeptide sequence but instead comprise an introduced polymer attachment site and optionally one or more additional mutations can be prepared using standard methodology. Those of ordinary skill in the art, familiar with standard molecular biology techniques, can employ that knowledge, coupled with the instant disclosure, to make and use these FGF21 variants polypeptides. Standard techniques can be used for recombinant DNA, oligonucleotide synthesis, tissue culture, and transformation (e.g., electroporation, lipofection). See, e.g., Sambrook et al., Molecular Cloning: A Laboratory Manual, which is incorporated herein by reference for any purpose. Enzymatic reactions and purification techniques can be performed according to manufacturer's specifications, as commonly accomplished in the art, or as described herein. Unless specific definitions are provided, the nomenclatures utilized in connection with, and the laboratory procedures and techniques of, analytical chemistry, synthetic organic chemistry, and medicinal and pharmaceutical chemistry described herein are those well known and commonly used in the art. Standard techniques can be used for chemical syntheses; chemical analyses; pharmaceutical preparation, formulation, and delivery; and treatment of patients.

Following the preparation of a FGF21 variant which contains neither of the cysteines found in the wild-type FGF21 polypeptide sequence but instead comprises an introduced polymer attachment site and optionally one or more additional mutations, the polypeptide can be chemically modified by the attachment of a polymer using standard methodology known to those of skill in the art, which will depend on the nature of the polymer being attached. See, e.g., U.S. Pat. Nos. 4,640,835; 4,496,689; 4,301,144; 4,670,417; 4,791,192; and 4,179,337.

In a further aspect, additional mutations can be introduced into a mature FGF21 sequence which can provide a site for GalNAc transferase-mediated glycosylation in which a GalNAc is added and serves as a point for O-glycosylation. The following list of mutations includes both point mutants as well as sequences of consecutive and non-consecutive mutations, and a GalNAc will be added to an S or a T residue. Examples of mutations that can provide a site for GalNAc transferase-mediated glycosylation of FGF21 include those shown in Table 8; in Table 8, when sequences of multiple amino acids are provided, the point mutants are highlighted in bold and are underlined (positions refer to the mature FGF21 polypeptide of SEQ ID NO:4 or 8):

TABLE 8
GalNAc Transferase-mediated Glycosylation Mutants

| | | Introduced Glycosylation |
|---|---|---|
| | Wild- | Site (Mutation |
| Position | Type Residue | Underlined) |
| 1 | H | VT |
| | | QT |
| | | AT |
| | | IAT |
| 1, 3 | H, I | F,T |
| | | A,T |
| | | F,S |
| | | A,S |
| | | S,T |
| 3 | I | T,S |
| 5-7 | DSS | TQA |
| | | TAQ |
| | | TIE |
| 5 | D | S,T |
| 9-12 | LLQF | TTQF |
| | | TINT |
| | | TQGA |
| | | TQGF |
| | | TTVS |
| | | TQAF |
| 45-50 | ADQSPE | ATQSPE |
| | | ATESPE |
| | | ATETPE |
| | | VTQSPE |
| | | VTETPE |
| | | ATESPA |
| 50-53 | ESLL | TSLL |
| | | TTVS |
| | | TINT |
| | | TQAL |
| | | TQGA |
| 59-64 | KPGVIQ | SPTVIQ |
| | | APTVIQ |
| | | SPTTVS |
| | | SPTINT |
| | | SPTQAQ |
| | | SPTQGA |
| | | SPTVIA |
| | | APTTVS |
| | | APTINT |
| 77-83 | RPDGAL | SPTGALY |
| | | APTGALY |
| | | SPTINTY |
| | | SPTTVSY |
| | | SPTQALY |
| | | APTQALY |
| | | SPTQGAY |
| | | SPTQGAM |
| 85-89 | SLHFD | SLTFT |
| | | SLTET |
| | | SVTET |
| 112 | H | T |
| 111-114 | AHGL | ATGT |
| | | ATET |
| | | VTET |
| | | ATGL |
| 116-118 | LHL | TQA |
| | | TAQ |
| | | TEI |
| | | TSS |
| | | TAL |
| 112-118 | HGLPLHLP | SGLPTQA |
| | | SGLPTEI |
| 120-125 | GNKSPH | TTAVPH |
| | | TSGEPH |
| | | GSTAPH |
| | | GNSTPH |
| | | GTESPH |
| | | LTQTPH |
| | | LTQTPA |
| | | TNASPH |
| | | TQGSPH |
| | | VTSQPH |
| | | TINTPH |
| | | TSVSPH |
| 122-131 | KSPHRDPAPR | KSPTAQPAPR |
| | | KSPTADPAPR |
| | | ASPTAQPAPR |
| | | SSPTADPAPR |
| | | KSPTSDPAPR |
| | | KSPTEIPAPR |
| | | KSPTEDPAPR |
| | | ASPTEDPAPR |
| | | SSPTADPAPR |
| | | SSPTAQPAPR |
| | | KSPTQAPAPR |
| | | SSPTQAPAPR |
| | | ASPTEIPAPR |
| | | KSPHRDPTPR |
| | | KSPHRDPTPA |
| | | KSPHRDPSPR |
| | | KSPHSDPTPA |
| | | KSPHADPTPS |
| | | KSPHADPTPA |
| 131-137 | RGPARFL | RGPTSFL |
| | | RGPTSGE |
| | | RGPGSTA |
| | | RGPANTS |
| | | RGPATES |
| | | RGPATQT |
| | | RGPLTQT |
| | | RGPTQFL |
| | | RGPTSFL |
| | | RGPVTSQ |
| | | SGPTSFL |
| | | AGPTSGE |
| | | SGPTSAL |
| 135-139 | RFLPL | RFLPT |
| | | RFLPS |
| | | SFLPT |
| 148 | E | T,S |
| 151 | G | T |
| 151-156 | GILAPQ | TTLAPQ |
| | | TQLAPQ |
| | | TSGEPQ |
| | | GSTAPQ |
| | | TTAVPQ |
| | | GNTSPQ |
| | | GTESPQ |
| | | GTETPQ |
| | | VTSQPQ |
| | | LTQTPQ |
| | | VTSQPQ |
| | | SSGAPQ |
| | | TINTPQ |
| | | TTVSPQ |
| | | TQAAPQ |
| | | GILAPT |
| | | GILAPS |
| 156 | Q | T,S |
| 159 | D | T |
| 159-164 | DVGSSD | DVGTET |
| | | DAASAA |
| | | DAATAA |
| | | DVGTSD |
| | | DVATSD |
| | | TGDSSD |
| | | TDASGA |
| | | DVGTSG |
| 164 | D | T |
| 166 | L | T |
| 166-170 | LSMVGP | TSGAM |
| | | TQGAM |
| | | TQGAM |
| 172-176 | SQGRS | SQGAS |
| | | TQGAS |
| | | TQGAM |
| 175 | R | A |
| 175-181 | RSPSYAS | RSPTSAVAA |
| | | ASPTSAVAA |
| | | ASPSSGAPPPS |
| | | ASPSSGAPP |
| | | ASPSSGAP |
| | | RSPSSGAPPPS |
| | | ASPTINT |
| | | ASPTSVS |
| | | ASPTQAF |
| | | ASPTINTP |

In contrast to Table 8, additional mutations can be introduced into a mature FGF21 sequence which can provide a reduced capacity for O-glycosylation, relative to the wild-type FGF21 sequence, when a FGF21 polypeptide or FGF21 variant is expressed in yeast. The list of mutations in Table 9 includes both point mutants as well as sequences of consecutive and non-consecutive mutations (positions refer to the mature FGF21 polypeptide of SEQ ID NO:4 or 8). Examples of mutations that can provide for reduced O-glycosylation, relative to the wild-type FGF21 sequence, when the FGF21 sequence is expressed in yeast include the S167A, S167E, S167D, S167N, S167Q, S167G, S167V, S167H, S167K and S167Y.

TABLE 9
O-Glycosylation Resistant Mutants

| Position | Wild-type | O-glycosylation Mutant |
|---|---|---|
| 167 | S | A, E, D, N, Q, G, V, H, K, Y |

In another aspect of the instant disclosure, the desirable properties of several FGF21 variants disclosed herein can be combined in an additive or synergistic fashion to generate an FGF21 variant exhibiting enhanced pharmaceutical properties. Thus, in another embodiment, the point mutations provided in Tables 1-13 can be combined to provide a desired profile for a variant FGF21 sequence.

As with all FGF21 mutants of the present invention, the FGF21 variants comprising two or more mutations of the present invention can be prepared as described herein. Those of ordinary skill in the art, familiar with standard molecular biology techniques, can employ that knowledge, coupled with the instant disclosure, to make and use the FGF21 variants comprising two or more mutations of the present invention. Standard techniques can be used for recombinant DNA, oligonucleotide synthesis, tissue culture, and transformation (e.g., electroporation, lipofection). See, e.g., Sambrook et al., Molecular Cloning: A Laboratory Manual, supra, which is incorporated herein by reference for any purpose. Enzymatic reactions and purification techniques can be performed according to manufacturer's specifications, as commonly accomplished in the art, or as described herein. Unless specific definitions are provided, the nomenclatures utilized in connection with, and the laboratory procedures and techniques of, analytical chemistry, synthetic organic chemistry, and medicinal and pharmaceutical chemistry described herein are those well known and commonly used in the art. Standard techniques can be used for chemical syntheses; chemical analyses; pharmaceutical preparation, formulation, and delivery; and treatment of patients.

The FGF21 variants comprising two or more mutations of the present invention can be fused to another entity, which can impart additional properties to a FGF21 variant comprising two or more mutations. In one embodiment of the present invention, a FGF21 variant comprising two or more mutations can be fused to an IgG Fc sequence. Such fusion can be accomplished using known molecular biological methods and/or the guidance provided herein. The benefits of such fusion polypeptides, as well as methods for making such fusion polypeptides, are discussed in more detail herein.

Examples of mutations that can be introduced into an FGF21 sequence either as a point mutation or as a combination of two or more point mutants are provided in Tables 1-13, specific examples of which are provided in Table 10 below (positions refer to the mature FGF21 polypeptide of SEQ ID NO:4 or 8):

TABLE 10
Summarized FGF21 Point Mutations

| | | | | | | Disulfide | | |
|---|---|---|---|---|---|---|---|---|
| | | | | | | Bonds | | |
| | | | | | | Between | | |
| | | | | | | Cys | Reduced C- | |
| | | | | | | Residues | Term | Reduced |
| | Wild | Introduced | | Proteolysis | Aggregation | at | Degradation | Glycosylation |
| Position | type | Cysteines | Stability Mutants | Mutants | Mutants | Positions | Mutants | Mutants |
| 1 | H | | | | | | | |
| 2 | P | | | | | | | |
| 3 | I | | | | | | | |
| 4 | P | | | | | | | |
| 5 | D | | | | | | | |
| 6 | S | | | | | | | |
| 7 | S | | | | | | | |
| 8 | P | | | | | | | |
| 9 | L | | | | | | | |
| 10 | L | | | | | | | |
| 11 | Q | | | | | | | |
| 12 | F | | | | | | | |
| 13 | G | | | | | | | |
| 14 | G | | | | | | | |
| 15 | Q | | | | | | | |
| 16 | V | | | | | | | |
| 17 | R | | | | | | | |
| 18 | Q | C | | | | | | |
| 19 | R | C | | Q, I, K | | 138 | | |
| 20 | Y | C | | H, L, F | | 139 | | |
| 21 | L | C | | I, F, Y, V | | 33 | | |
| 22 | Y | C | | I, F, V | | 137, 139 | | |
| 23 | T | C | | | | 25, 28 | | |
| 24 | D | C | | | | 135 | | |
| 25 | D | C | | | | 23, 122 | | |
| 26 | A | C | | | E, K, R | 122 | | |
| 27 | Q | C | | | | 123 | | |
| 28 | Q | C | | | | 28, 43, | | |
| | | | | | | 124 | | |
| 29 | T | C | | | | | | |
| 30 | E | C | | | | | | |
| 31 | A | C | | | | 43 | | |
| 32 | H | | | | | | | |
| 33 | L | C | | | | 21 | | |
| 34 | E | | | | | | | |
| 35 | I | C | | | | 84 | | |
| 36 | R | C | | | | | | |
| 37 | E | C | | | | | | |
| 38 | D | C | | | | | | |
| 39 | G | C | | | | | | |
| 40 | T | C | | | | 82 | | |
| 41 | V | C | | | | | | |
| 42 | G | C | D, E, R, K, H, | | | 124, 126 | | |
| | | | S, T, N, Q | | | | | |
| 43 | G | C | | | | 28, 31, | | |
| | | | | | | 124 | | |
| 44 | A | C | | | | | | |
| 45 | A | C | | | E, K, R, Q, T | | | |
| 46 | D | C | | | | | | |
| 47 | Q | C | | | | | | |
| 48 | S | C | | | | | | |
| 49 | P | C | | | | | | |
| 50 | E | C | | | | 69 | | |
| 51 | S | | | | | | | |
| 52 | L | | | | T | | | |
| 53 | L | | | | | | | |
| 54 | Q | C | D, E, R, K, H, | | | 66 | | |
| | | | S, T, N, Q | | | | | |
| 55 | L | | | | | | | |
| 56 | K | C | | | | | | |
| 57 | A | C | | | | | | |
| 58 | L | C | | | C, E, S | 62 | | |
| 59 | K | C | | | | | | |
| 60 | P | C | | | A, E, K, R | | | |
| 61 | G | C | | | | | | |
| 62 | V | C | | | | 58 | | |
| 63 | I | | | | | | | |
| 64 | Q | C | | | | | | |
| 65 | I | C | | | | | | |
| 66 | L | C | | | | 54 | | |
| 67 | G | C | | | | 72, 135 | | |
| 68 | V | C | | | | | | |
| 69 | K | C | | | | 50 | | |
| 70 | T | C | | | | | | |
| 71 | S | C | | | | | | |
| 72 | R | C | | | | 67, 84 | | |
| 73 | F | C | | | | 93 | | |
| 74 | L | | | | | | | |
| 75 | C | C | | | | 85, 92 | | |
| 76 | Q | C | | | | 109 | | |
| 77 | R | C | D, E, R, K, H, | | | 79, 81 | | |
| | | | S, T, N, Q | | | | | |
| 78 | P | C | | | A, C, H, R | | | |
| 79 | D | C | | | | 77 | | |
| 80 | G | C | | | | 129 | | |
| 81 | A | C | D, E, R, K, H, | | | 77 | | |
| | | | S, T, N, Q | | | | | |
| 82 | L | C | | | | 41, 119 | | |
| 83 | Y | C | | | | | | |
| 84 | G | C | | | | 35, 72 | | |
| 85 | S | C | | | | 75 | | |
| 86 | L | C | D, E, R, K, H, | | C, T | | | |
| | | | S, T, N, Q | | | | | |
| 87 | H | C | | | | | | |
| 88 | F | C | D, E, R, K, H, | | A, E, K, R, S | | | |
| | | | S, T, N, Q | | | | | |
| 89 | D | C | | | | | | |
| 90 | P | C | | | | 92 | | |
| 91 | E | C | | | | | | |
| 92 | A | C | | | | 90 | | |
| 93 | C | C | | | | 73 | | |
| 94 | S | C | | | | 110 | | |
| 95 | F | C | | | | 107 | | |
| 96 | R | | G, A, V, P, F, | | | | | |
| | | | Y, W, S, T, | | | | | |
| | | | N, D, Q, E, | | | | | |
| | | | C, M | | | | | |
| 97 | E | | | | | | | |
| 98 | L | C | | | C, E, K, Q, R | | | |
| 99 | L | C | | | C, D, E, R | | | |
| 100 | L | C | | | | 102 | | |
| 101 | E | C | | | | | | |
| 102 | D | C | | | | 100, 104 | | |
| 103 | G | C | | | | | | |
| 104 | Y | C | | | | 102 | | |
| 105 | N | | | | | | | |
| 106 | V | C | | | | | | |
| 107 | Y | C | | | | 95 | | |
| 108 | Q | C | | | | | | |
| 109 | S | C | | | | 76 | | |
| 110 | E | C | | | | 94 | | |
| 111 | A | C | | | K, T | | | |
| 112 | H | C | | | | | | |
| 113 | G | C | | | | | | |
| 114 | L | C | | | | | | |
| 115 | P | C | | | | 117 | | |
| 116 | L | C | | | | | | |
| 117 | H | C | | | | 115, | | |
| | | | | | | 129, 130 | | |
| 118 | L | C | | | | 132, 134 | | |
| 119 | P | C | | | | 82 | | |
| 120 | G | C | | | | | | |
| 121 | N | C | D, S, A, V, | | | 127 | | |
| | | | S, E | | | | | |
| 122 | K | C | D, E, R, K, H, | | | 25, 26 | | |
| | | | S, T, N, Q | | | | | |
| 123 | S | C | | | | 27, 125 | | |
| 124 | P | C | | | | 28, 42, | | |
| | | | | | | 43 | | |
| 125 | H | C | D, E, R, K, | | | 123 | | |
| | | | H, S, T, N, Q | | | | | |
| 126 | R | C | D, E, R, K, | | | 42 | | |
| | | | H, S, T, N, Q | | | | | |
| 127 | D | C | | | | 121, 132 | | |
| 128 | P | C | | | | | | |
| 129 | A | C | | | D, E, H, K, | 80, 117 | | |
| | | | | | N, R, Q | | | |
| 130 | P | C | D, E, R, K, | | | 117 | | |
| | | | H, S, T, N, Q | | | | | |
| 131 | R | C | S, T, N, Q, | | | | | |
| | | | D, E, R, K, H | | | | | |
| 132 | G | C | | | | 118, 127 | | |
| 133 | P | C | | | | | | |
| 134 | A | C | | | E, H, KY | 118 | | |
| 135 | R | C | | | | 24, 67 | | |
| 136 | F | | | | | | | |
| 137 | L | C | | | | 22 | | |
| 138 | P | C | | | | 19 | | |
| 139 | L | C | D, E, R, K, | | | 20, 22 | | |
| | | | H, S, T, N, Q | | | | | |
| 140 | P | C | | | | | | |
| 141 | G | | | | | | | |
| 142 | L | | | | | | | |
| 143 | P | | | | | | | |
| 144 | P | | | | | | | |
| 145 | A | | D, E, R, K, H, | | | | | |
| | | | S, T, N, Q | | | | | |
| 146 | P | | D, E, R, K, H, | | | | | |
| | | | S, T, N, Q | | | | | |
| 147 | P | | | | | | | |
| 148 | E | | | | | | | |
| 149 | P | | | | | | | |
| 150 | P | | | A, R | | | | |
| 151 | G | | | A, V | | | | |
| 152 | I | C | D, E, R, K, | H, L, F, V | | 163 | | |
| | | | H, S, T, N, Q | | | | | |
| 153 | L | C | G, A, V, P, | | | | | |
| | | | F, Y, W, S, | | | | | |
| | | | T, N, D, Q, | | | | | |
| | | | E, C, M, I | | | | | |
| 154 | A | C | V, P, F, Y, | | | | | |
| | | | W, C, M, L, | | | | | |
| | | | D, E, R, K, | | | | | |
| | | | H, S, T, N, Q | | | | | |
| 155 | P | | | | | | | |
| 156 | Q | | D, E, R, K, | | | | | |
| | | | H, S, T, N, Q | | | | | |
| 157 | P | | | | | | | |
| 158 | P | | | | | | | |
| 159 | D | | | | | | | |
| 160 | V | | | | | | | |
| 161 | G | | D, E, R, K, | | | | | |
| | | | H, S, T, N, Q | | | | | |
| 162 | S | | | | | | | |
| 163 | S | C | D, E, R, K, | | | 152 | | |
| | | | H, S, T, N, Q | | | | | |
| 164 | D | | | | | | | |
| 165 | P | | | | | | | |
| 166 | L | | | | | | | |
| 167 | S | C | A, E, D, N, | | | | | A, E, D, N, Q, G, V, |
| | | | Q, G, V, H, | | | | | H, K, Y |
| | | | K, Y, F, W, | | | | | |
| | | | M, R, C, I, L, P | | | | | |
| 168 | M | | | | | | | |
| 169 | V | | | | | | | |
| 170 | G | | D, E, R, K, | A, N, D, C, | | | | |
| | | | H, S, T, N, Q | Q, E, P, S | | | | |
| 171 | P | | | A, R, N, D, | | | | |
| | | | | C, E, Q, G, | | | | |
| | | | | H, K, S, T, | | | | |
| | | | | W, Y | | | | |
| 172 | S | | D, E, R, K, | L, T | | | | |
| | | | H, S, T, N, Q | | | | | |
| 173 | Q | | | R, E | | | | |
| 174 | G | | A | | | | | |
| 175 | R | | A | | | | | |
| 176 | S | | | | | | | |
| 177 | P | | A | | | | | |
| 178 | S | | | | | | | |
| 179 | Y | | | | | | P, G, S, A | |
| 180 | A | | | | | | E, P, S | |
| 181 | S | | G | | | | G, P, K, T, A, L, P | |

In a particular embodiment a variant FGF21 polypeptide comprises the L98R mutation and the P171G mutation introduced into mature FGF21 comprising SEQ ID NO:4 or 8, provided herein as SEQ ID NO: 10. One specific example of such a variant includes the Fc fusion of SEQ ID NO:39, wherein the FGF21 sequence of SEQ ID NO:10 is joined to the Fc sequence of SEQ ID NO:47 via the linker of SEQ ID NO:33.

In another specific embodiment a variant FGF21 polypeptide comprises the L98R mutation, the P171G mutation and the A180E mutation introduced into mature FGF21 comprising SEQ ID NO:4 or 8, provided herein as SEQ ID NO:12. One specific example of such a variant includes the Fc fusion of SEQ ID NO:41, wherein the FGF21 sequence of SEQ ID NO: 12 is joined to the Fc sequence of SEQ ID NO:47 via the linker of SEQ ID NO:33.

Additional specific FGF21 variant polypeptides that can be employed in the disclosed methods are described in, e.g., WO 2010/042747, WO 2009/149171, WO 2010129503, incorporated herein by reference.

### IV. “Tethered Molecules”

In still another aspect of the present invention, a “Tethered Molecule” can be employed in the disclosed methods. Such “Tethered Molecules” can be prepared as described herein. A “Tethered Molecule” is a molecule comprising two wild-type FGF21 polypeptides tethered together (e.g., SEQ ID NO:4 or 8 or a combination thereof) by a linker molecule. By joining two FGF21 polypeptides or two FGF21 variants or a wild-type FGF21 polypeptide and a FGF21 variant together, the effective half-life and potency of a Tethered Molecule can be extended beyond the half-life and potency of a single FGF21 polypeptide or variant.

A Tethered Molecule of the present invention comprises a linker and two wild-type FGF21 polypeptides or FGF21 variants or a combination thereof, and can comprise two naturally occurring FGF21 polypeptides into which no mutations have been introduced, two FGF21 mutant polypeptides having a linker attachment site introduced into the FGF21 polypeptides or a combination of one naturally occurring FGF21 polypeptide and one FGF21 variant. Tethered Molecules comprising at least one FGF21 polypeptide or FGF21 variant having a non-naturally occurring linker attachment site and one or more additional mutations are also contemplated and form another aspect of the invention. Such Tethered Molecules can thus comprise a mutation that forms a site for the attachment of a linker molecule as well as another mutation to impart another desirable property to the Tethered Molecule.

As used herein, the term “linker attachment site” means a naturally or non-naturally occurring amino acid having a functional group with which a linker can be associated. In one example, a linker attachment site is a residue containing a thiol group, which can be associated with a PEG molecule.

### IV.A. FGF21 Polypeptides and FGF21 Variants in a Tethered Molecule

When a Tethered Molecule comprises two FGF21 variants, the FGF21 variants can comprise one or more mutations introduced into the sequence, but the mutations need not be at the same amino acid position in each of the FGF21 variant polypeptides. By way of example, if a Tethered Molecule comprises two FGF21 variant polypeptides, one FGF21 mutant polypeptide may contain an H125C mutation, which may form an attachment point for a linker molecule. In contrast, the other FGF21 variant polypeptide can contain a mutation at a position other than H125 which can serve as an attachment point for the linker tethering the two FGF21 variant polypeptides together. Even if one or two FGF21 variant polypeptides are employed, the linker can be attached at the N terminal end of the FGF21 variant polypeptide; introduced attachment points need not necessarily be used.

When a Tethered Molecule comprises one or two naturally-occurring wild-type FGF21 polypeptides (e.g., SEQ ID NO:4 or 8 or a combination thereof) the linker can be attached at a point in the FGF21 polypeptide that is amenable to the attachment chemistry. For example, naturally occurring disulfide bonds can be reduced and the cysteine residues can serve as attachment points for a linker, such as PEG. In another embodiment, a linker can be attached to a FGF21 polypeptide at the N-terminus or on lysine sidechains.

One or both of the FGF21 variant polypeptides of a Tethered Molecule can comprise a truncated FGF21 variant polypeptide. As described herein, a truncated FGF21 variant polypeptide can be prepared by removing any number of residues on either the N-terminus, the C-terminus or both the N- and C-termini.

Tethered Molecules can also comprise one or both FGF21 polypeptides which comprise a mutation in the polypeptide sequence that may not be preferred as a linker attachment site, but instead may impart some other desirable property to the Tethered Molecule (e.g., those mutations described in Tables 1-13). Thus, Tethered Molecules comprising one or more FGF21 variant polypeptides into which a mutation imparting a desirable property to the Tethered Molecule form a further aspect of the present invention.

The activity of Tethered Molecules can be assayed in a variety of ways, for example, using an in vitro ELK-luciferase assay as described herein.

The activity of all of the disclosed FGF21 polypeptide and FGF21 variants disclosed herein, including the disclosed Tethered Molecules, can also be assessed in an in vivo assay, such as with ob/ob mice. Generally, to assess the in vivo activity of one or more of these polypeptides, the polypeptide can be administered to a test animal intraperitoneally. After one or more desired time periods, a blood sample can be drawn, and a biomarker, such as the level of insulin, cholesterol, lipid or blood glucose, can be measured.

As is the case for all FGF21 polypeptide and FGF21 variants of the present invention, the FGF21 polypeptides that comprise a Tethered Molecule, which can be FGF21 variant polypeptides, wild-type FGF21 polypeptides or a combination of both, can optionally comprise an amino-terminal methionine residue, which can be introduced by directed mutation or as a result of a bacterial expression process.

Those of ordinary skill in the art, familiar with standard molecular biology techniques, can employ that knowledge, coupled with the instant disclosure, to make and use the Tethered Molecules (and all of the FGF21 polypeptides and FGF21 variants) provided herein. Standard techniques can be used for recombinant DNA, oligonucleotide synthesis, tissue culture, and transformation (e.g., electroporation, lipofection). See, e.g., Sambrook et al., Molecular Cloning: A Laboratory Manual, which is incorporated herein by reference for any purpose. Enzymatic reactions and purification techniques can be performed according to manufacturer's specifications, as commonly accomplished in the art, or as described herein. Processes for associating linkers with FGF21 polypeptides and FGF21 variants will depend on the nature of the linker, but are known to those of skill in the art. Examples of linker attachment chemistries are described herein.

Unless specific definitions are provided, the nomenclatures utilized in connection with, and the laboratory procedures and techniques of, analytical chemistry, synthetic organic chemistry, and medicinal and pharmaceutical chemistry described herein are those well known and commonly used in the art. Standard techniques can be used for chemical syntheses; chemical analyses; pharmaceutical preparation, formulation, and delivery; and treatment of patients.

### IV.B. Linkers Useful for Forming Tethered Molecules

Any linker can be employed in a Tethered Molecule to tether two FGF21 polypeptides or FGF21 variant polypeptides together. Linker molecules can be branched or unbranched and can be attached to a FGF21 variant polypeptide using various known chemistries, such as those described herein. The chemical structure of a linker is not critical, since it serves primarily as a spacer. The linker can be independently the same or different from any other linker, or linkers, that may be present in a Tethered Molecule (e.g., a Tethered Molecule comprising three or more FGF21 variant or FGF21 polypeptides). In one embodiment, a linker can be made up of amino acids linked together by peptide bonds. Some of these amino acids can be glycosylated, as is well understood by those in the art. For example, a useful linker sequence constituting a sialylation site is X₁X₂NX₃X₄G (SEQ ID NO:46, wherein X₁, X₂, X₄ and X₅ are each independently any amino acid residue. In another embodiment a linker molecule can be a PEG molecule of any size, such as 20 kDa, 30 kDa or 40 kDa.

In embodiments in which a peptidyl linker is present (i.e., made up of amino acids linked together by peptide bonds) that is made in length, preferably, of from 1 up to about 40 amino acid residues, more preferably, of from 1 up to about 20 amino acid residues, and most preferably of from 1 to about 10 amino acid residues. In one embodiment, the amino acid residues in the linker are selected from any the twenty canonical amino acids. In another embodiment the amino acid residues in the linker are selected from cysteine, glycine, alanine, proline, asparagine, glutamine, and/or serine. In yet another embodiment, a peptidyl linker is made up of a majority of amino acids that are sterically unhindered, such as glycine, serine, and alanine linked by a peptide bond. It is often desirable that, if present, a peptidyl linker be selected that avoids rapid proteolytic turnover in circulation in vivo. Thus, preferred peptidyl linkers include polyglycines, particularly (Gly)₄ (SEQ ID NO: 13); (Gly)₅ (SEQ ID NO: 14); poly(Gly-Ala); and polyalanines. Other preferred peptidyl linkers include GGGGS (SEQ ID NO:15); GGGGSGGGGS (SEQ ID NO:16); GGGGSGGGGSGGGGSGGGGSGGGGS (SEQ ID NO: 17) and any linkers used in the Examples provided herein. The linkers described herein, however, are exemplary; linkers within the scope of this invention can be much longer and can include other residues.

In embodiments of a Tethered Molecule that comprise a peptide linker moiety, acidic residues, for example, glutamate or aspartate residues, are placed in the amino acid sequence of the linker moiety. Examples include the following peptide linker sequences:

(SEQ ID NO: 18);
GGEGGG
(SEQ ID NO: 19);
GGEEEGGG
(SEQ ID NO: 20);
GEEEG
(SEQ ID NO: 21);
GEEE
(SEQ ID NO: 22);
GGDGGG
(SEQ ID NO: 23);
GGDDDGG
(SEQ ID NO: 24);
GDDDG
(SEQ ID NO: 25);
GDDD
(SEQ ID NO: 26);
GGGGSDDSDEGSDGEDGGGGS
(SEQ ID NO: 27);
WEWEW
(SEQ ID NO: 28);
FEFEF
(SEQ ID NO: 29);
EEEWWW
(SEQ ID NO: 30);
EEEFFF
(SEQ ID NO: 31); or
WWEEEWW
(SEQ ID NO: 32).
FFEEEFF

In other embodiments, a peptidyl linker constitutes a phosphorylation site, e.g., X₁X₂YX₃X₄G (SEQ ID NO:43), wherein X₁, X₂, X₃ and X₄ are each independently any amino acid residue; X₁X₂SX₃X₄G (SEQ ID NO:44), wherein X₁, X₂, X₃ and X₄ are each independently any amino acid residue; or X₁X₂TX₃X₄G (SEQ ID NO:45), wherein X₁, X₂, X₃ and X₄ are each independently any amino acid residue.

Non-peptide linkers can also be used in a Tethered Molecule. For example, alkyl linkers such as —NH—(CH₂)ₛ—C(O)—, wherein s=2 to 20 could be used. These alkyl linkers can further be substituted by any non-sterically hindering group such as lower alkyl (e.g., C₁-C₆) lower acyl, halogen (e.g., Cl, Br), CN, NH₂, phenyl, etc.

Any suitable linker can be employed in the present invention to form Tethered Molecules. In one example, the linker used to produce Tethered Molecules described herein were homobifunctional bis-maleimide PEG molecules having the general structure:

X—(CH₂CH₂O)ₙCH₂CH₂—X

where X is a maleimide group. In other embodiments, X can be an orthopyridyl-disulphide, an iodoacetamide, a vinylsulfone or any other reactive moiety known to the art to be specific for thiol groups. In yet another embodiment X can be an amino-specific reactive moiety used to tether two mutant polypeptides through either the N-terminus or an engineered lysyl group. (See, e.g., Pasut and Veronese, 2006, “PEGylation of Proteins as Tailored Chemistry for Optimized Bioconjugates,” Adv. Polym. Sci. 192:95-134).

In still another embodiment, a linker can have the general structure:

X—(CH₂CH₂O)ₙCH₂CH₂—Y

where X and Y are different reactive moieties selected from the groups above. Such a linker would allow conjugation of different mutant polypeptides to generate Tethered heterodimers or hetero-oligomers.

In a further embodiment, a linker can be a PEG molecule, which can have a molecular weight of 1 to 100 kDa, preferably 10 to 50 kDa (e.g., 10, 20, 30 or 40 kDa) and more preferably 20 kDa. The peptide linkers can be altered to form derivatives in the same manner as described above.

Other examples of useful linkers include aminoethyloxyethyloxy-acetyl linkers as disclosed in International Publication No. WO 2006/042151, incorporated herein by reference in its entirety.

When forming a Tethered Molecule of the present invention, standard chemistries can be employed to associate a linker with a FGF21 polypeptide or variant FGF21 polypeptide. The precise method of association will depend on the attachment site (e.g., which amino acid side chains) and the nature of the linker. When a linker is a PEG molecule, attachment can be achieved by employing standard chemistry and a free sufhydryl or amine group, such as those found on cysteine residues (which can be introduced into the FGF21 polypeptide or FGF21 variant polypeptide sequence by mutation or can be naturally occurring) or on lysine (which can be introduced into the FGF21 sequence by mutation or can be naturally occurring) or N-terminal amino groups.

### V. Chemically-Modified FGF21 Mutants

Chemically modified forms of the FGF21 polypeptides and FGF21 variants described herein, including the truncated forms of the FGF21 molecules described herein, can be prepared by one skilled in the art, given the disclosures described herein. Such chemically modified FGF21 polypeptides and variants are altered such that the chemically modified FGF21 polypeptide or FGF21 variant is different from the unmodified FGF21 polypeptide, either in the type or location of the molecules naturally attached to the FGF21 variant. Chemically modified FGF21 polypeptides and FGF21 variants can include molecules formed by the deletion of one or more naturally-attached chemical groups.

Additional FGF21 variants that can be suitable for chemical modification include those of Table 11, which provides individual point mutations that can serve as attachment/reaction points for chemical modification. The residue numbers provided are relative to a mature FGF21 polypeptide (e.g., SEQ ID NO:4 or 8).

TABLE 11
FGF21 Variant Polypeptides Comprising a Single Mutation

| Residue Number | WT | Mutation | |
|---|---|---|---|
| 36 | R | K | R36K |
| 37 | E | C | E37C |
| 38 | D | C | D38C |
| 46 | D | C | D46C |
| 56 | K | R | K56R |
| 60 | K | R | K60R |
| 91 | E | C | E91C |
| 69 | K | C | K69C |
| 69 | K | R | K69R |
| 72 | R | K | R72K |
| 77 | R | C | R77C |
| 77 | R | K | R77K |
| 79 | D | C | D79C |
| 86 | H | C | H86C |
| 91 | E | C | E91C |
| 112 | H | C | H112C |
| 113 | G | C | G113C |
| 120 | G | C | G120C |
| 121 | N | C | N121C |
| 122 | K | R | K122R |
| 125 | H | C | H125C |
| 126 | R | C | R126C |
| 126 | R | K | R126K |
| 171 | P | G | P171G |
| 175 | R | C | R175C |
| 175 | R | K | R175K |
| 170 | G | C | G170C |
| 179 | Y | C | Y179C |

While Table 11 describes various single point mutations, multiple point mutations can be introduced into a FGF21 sequence to generate multiple sites for chemical modification, including those described in Table 11. Thus, additional FGF21 variants that can be suitable for chemical modification include those of Table 12, which provides combinations of point mutations that can serve as attachment/reaction points for chemical modification. The residue numbers provided are relative to a mature FGF21 polypeptide, (e.g., SEQ ID NO:4 or 8).

TABLE 12
FGF21 Variant Polypeptides Comprising Two Mutations

| Residue | | | Residue | | | |
|---|---|---|---|---|---|---|
| 1 | WT | Mutation | 2 | WT | Mutation | |
| 37 | E | C | 77 | R | C | E37C, R77C |
| 120 | G | C | 125 | H | C | G120C, H125C |
| 77 | R | C | 91 | E | C | R77C, E91C |
| 77 | R | C | 125 | H | C | R77C, H125C |
| 91 | E | C | 125 | H | C | E91C, H125C |
| 77 | R | C | 120 | G | C | R77C, G120C |
| 37 | E | C | 91 | E | C | E37C, E91C |
| 91 | E | C | 175 | R | C | E91C, R175C |
| 37 | E | C | 175 | R | C | E37C, R175C |
| 91 | E | C | 120 | G | C | E91C, G120C |
| 37 | E | C | 120 | G | C | E37C, G120C |
| 77 | R | C | 175 | R | C | R77C, R175C |
| 37 | E | C | 125 | H | C | E37C, H125C |
| 37 | E | C | 69 | K | C | E37C, K69C |
| 69 | K | C | 91 | E | C | K69C, E91C |
| 120 | G | C | 175 | R | C | G120C, R175C |
| 69 | K | C | 120 | G | C | K69C, G120C |
| 69 | K | C | 125 | H | C | K69C, H125C |
| 69 | K | C | 77 | R | C | K69C, R77C |
| 125 | H | C | 175 | R | C | H125C, R175C |
| 69 | K | C | 175 | R | C | K69C, R175C |
| 37 | E | C | 170 | G | C | E37C, G170C |

Table 11 describes various single point mutations, multiple point mutations can be introduced into a FGF21 sequence to generate multiple sites for chemical modification, and Table 12, provides combinations of two point mutations that can serve as attachment/reaction points for chemical modification. Table 13 provided below provides combinations of three point mutations that can serve as attachment/reaction points for chemical modification. The residue numbers provided are relative to a mature FGF21 polypeptide, (e.g., SEQ ID NO:4 or 8).

TABLE 13
FGF21 Variant Polypeptides Comprising Three Mutations

| Residue 1 | WT | Mutation | Residue 2 | WT | Mutation | Residue 3 | WT | Mutation | |
|---|---|---|---|---|---|---|---|---|---|
| 37 | E | C | 77 | R | C | 171 | P | G | E37C, |
| | | | | | | | | | R77C, |
| | | | | | | | | | P171G |
| 91 | E | C | 125 | H | C | 171 | P | G | E91C, |
| | | | | | | | | | H125C, |
| | | | | | | | | | P171G |
| 77 | R | C | 120 | G | C | 171 | P | G | R77C, |
| | | | | | | | | | G120C, |
| | | | | | | | | | P171G |
| 37 | E | C | 91 | E | C | 171 | P | G | E37C, |
| | | | | | | | | | E91C, |
| | | | | | | | | | P171G |
| 91 | E | C | 175 | R | C | 171 | P | G | E91C, |
| | | | | | | | | | R175C, |
| | | | | | | | | | P171G |
| 37 | E | C | 175 | R | C | 171 | P | G | E37C, |
| | | | | | | | | | R175C, |
| | | | | | | | | | P171G |
| 91 | E | C | 120 | G | C | 171 | P | G | E91C, |
| | | | | | | | | | G120C, |
| | | | | | | | | | P171G |
| 37 | E | C | 120 | G | C | 171 | P | G | E37C, |
| | | | | | | | | | G120C, |
| | | | | | | | | | P171G |
| 77 | R | C | 175 | R | C | 171 | P | G | R77C, |
| | | | | | | | | | R175C, |
| | | | | | | | | | P171G |
| 37 | E | C | 125 | H | C | 171 | P | G | E37C, |
| | | | | | | | | | H125C, |
| | | | | | | | | | P171G |

In one embodiment, FGF21 polypeptide variants of the present invention can be modified by the covalent attachment of one or more polymers. For example, the polymer selected is typically water-soluble so that the protein to which it is attached does not precipitate in an aqueous environment, such as a physiological environment. Included within the scope of suitable polymers is a mixture of polymers. Preferably, for therapeutic use of the end-product preparation, the polymer will be pharmaceutically acceptable. Non-water soluble polymers conjugated to FGF21 polypeptides and FGF21 variants provided herein also form an aspect of the disclosure.

Exemplary polymers each can be of any molecular weight and can be branched or unbranched. The polymers each typically have an average molecular weight of between about 2 kDa to about 100 kDa (the term “about” indicating that in preparations of a water-soluble polymer, some molecules will weigh more and some less than the stated molecular weight). The average molecular weight of each polymer is preferably between about 5 kDa and about 50 kDa, more preferably between about 12 kDa and about 40 kDa, and most preferably between about 20 kDa and about 35 kDa.

Suitable water-soluble polymers or mixtures thereof include, but are not limited to, N-linked or O-linked carbohydrates, sugars, phosphates, polyethylene glycol (PEG) (including the forms of PEG that have been used to derivatize proteins, including mono-(C₁-C₁₀), alkoxy-, or aryloxy-polyethylene glycol), monomethoxy-polyethylene glycol, dextran (such as low molecular weight dextran of, for example, about 6 kD), cellulose, or other carbohydrate based polymers, poly-(N-vinyl pyrrolidone) polyethylene glycol, propylene glycol homopolymers, polypropylene oxide/ethylene oxide co-polymers, polyoxyethylated polyols (e.g., glycerol), and polyvinyl alcohol. Also encompassed by the present invention are bifunctional crosslinking molecules that can be used to prepare covalently attached FGF21 polypeptide mutant multimers. Also encompassed by the present invention are FGF21 mutants covalently attached to polysialic acid.

In some embodiments of the instant disclosure, a FGF21 variant is covalently, or chemically, modified to include one or more water-soluble polymers, including, but not limited to, polyethylene glycol (PEG), polyoxyethylene glycol, or polypropylene glycol. See, e.g., U.S. Pat. Nos. 4,640,835; 4,496,689; 4,301,144; 4,670,417; 4,791,192; and 4,179,337 and the Examples provided herein. In some embodiments of the present invention, an FGF21 mutant comprises one or more polymers, including, but not limited to, monomethoxy-polyethylene glycol, dextran, cellulose, another carbohydrate-based polymer, poly-(N-vinyl pyrrolidone)-polyethylene glycol, propylene glycol homopolymers, a polypropylene oxide/ethylene oxide co-polymer, polyoxyethylated polyols (e.g., glycerol), polyvinyl alcohol, or mixtures of such polymers.

In some embodiments of the instant disclosure, a FGF21 polypeptide or FGF21 variant is covalently-modified with PEG subunits. In some embodiments, one or more water-soluble polymers are bonded at one or more specific positions (for example, at the N-terminus) of the FGF21 polypeptide or variant. In some embodiments, one or more water-soluble polymers are randomly attached to one or more side chains of an FGF21 polypeptide or FGF21 variant. In some embodiments, PEG is used to improve the therapeutic capacity of a FGF21 polypeptide or FGF21 variant, which can be desirable when practicing the disclosed methods. Certain methods are discussed, for example, in U.S. Pat. No. 6,133,426, which is hereby incorporated by reference for any purpose.

In embodiments of the instant disclosure wherein the polymer is PEG, the PEG group can be of any convenient molecular weight, and can be linear or branched. The average molecular weight of the PEG group will preferably range from about 2 kD to about 100 kDa, and more preferably from about 5 kDa to about 50 kDa, e.g., 10, 20, 30, 40, or 50 kDa. The PEG groups will generally be attached to the FGF21 mutant via acylation or reductive alkylation through a reactive group on the PEG moiety (e.g., an aldehyde, amino, thiol, or ester group) to a reactive group on the FGF21 polypeptide or FGF21 variant (e.g., an aldehyde, amino, or ester group).

The PEGylation of a polypeptide, including the FGF21 polypeptides and FGF231 variants of the instant disclosure, can be specifically carried out using any of the PEGylation reactions known in the art. Such reactions are described, for example, in the following references: Francis et al., 1992, Focus on Growth Factors 3: 4-10; European Patent Nos. 0 154 316 and 0 401 384; and U.S. Pat. No. 4,179,337. For example, PEGylation can be carried out via an acylation reaction or an alkylation reaction with a reactive polyethylene glycol molecule (or an analogous reactive water-soluble polymer) as described herein. For the acylation reactions, a selected polymer should have a single reactive ester group. For reductive alkylation, a selected polymer should have a single reactive aldehyde group. A reactive aldehyde is, for example, polyethylene glycol propionaldehyde, which is water stable, or mono C₁-C₁₀ alkoxy or aryloxy derivatives thereof (see, e.g., U.S. Pat. No. 5,252,714).

In some embodiments of the instant disclosure, a useful strategy for the attachment of the PEG group to a polypeptide involves combining, through the formation of a conjugate linkage in solution, a peptide and a PEG moiety, each bearing a special functionality that is mutually reactive toward the other. The peptides can be easily prepared with conventional solid phase synthesis. The peptides are “preactivated” with an appropriate functional group at a specific site. The precursors are purified and fully characterized prior to reacting with the PEG moiety. Ligation of the peptide with PEG usually takes place in aqueous phase and can be easily monitored by reverse phase analytical HPLC. The PEGylated peptides can be easily purified by preparative HPLC and characterized by analytical HPLC, amino acid analysis and laser desorption mass spectrometry.

Polysaccharide polymers are another type of water-soluble polymer that can be used for protein modification. Therefore, the FGF21 polypeptides and FGF21 variants disclosed herein fused to a polysaccharide polymer form additional embodiments of FGF21 polypeptides and FGF21 variants that can be employed in the disclosed methods. Dextrans are polysaccharide polymers comprised of individual subunits of glucose predominantly linked by alpha 1-6 linkages. The dextran itself is available in many molecular weight ranges, and is readily available in molecular weights from about 1 kD to about 70 kD. Dextran is a suitable water-soluble polymer for use as a vehicle by itself or in combination with another vehicle (e.g., Fc). See, e.g., International Publication No. WO 96/11953. The use of dextran conjugated to therapeutic or diagnostic immunoglobulins has been reported. See, e.g., European Patent Publication No. 0 315 456, which is hereby incorporated by reference. The present invention also encompasses the use of dextran of about 1 kD to about 20 kD.

In general, chemical modification can be performed under any suitable condition used to react a protein with an activated polymer molecule. Methods for preparing chemically modified polypeptides will generally comprise the steps of: (a) reacting the polypeptide with the activated polymer molecule (such as a reactive ester or aldehyde derivative of the polymer molecule) under conditions whereby a FGF21 polypeptide or FGF21 variant becomes attached to one or more polymer molecules, and (b) obtaining the reaction products. The optimal reaction conditions will be determined based on known parameters and the desired result. For example, the larger the ratio of polymer molecules to protein, the greater the percentage of attached polymer molecule. In one embodiment of the present invention, chemically modified FGF21 polypeptides and FGF21 variants can have a single polymer molecule moiety at the amino-terminus (see, e.g., U.S. Pat. No. 5,234,784)

In another embodiment of the present invention, a FGF21 polypeptide or variant can be chemically coupled to biotin. The biotin/FGF21 polypeptide or variant is then allowed to bind to avidin, resulting in a tetravalent avidin/biotin/FGF21 polypeptide variant. FGF21 polypeptides and FGF21 variants can also be covalently coupled to dinitrophenol (DNP) or trinitrophenol (TNP) and the resulting conjugates precipitated with anti-DNP or anti-TNP-IgM to form decameric conjugates with a valency of 10.

Generally, conditions that can be alleviated or modulated by the administration of the disclosed chemically modified FGF21 polypeptides and FGF21 variants include those described herein, e.g., Type 1 diabetes, and thus can be employed in the disclosed methods. However, the chemically modified FGF21 variants disclosed herein can also have additional activities, enhanced or reduced biological activity, or other characteristics, such as increased or decreased half-life, as compared to unmodified FGF21 variants.

VI. Molecules that Exhibit FGF21-Like Signaling

It is noted that while a range of FGF21 polypeptides and FGF21 variants that can be useful in carrying out the disclosed methods have been provided in Tables 1-13, it is noted that these molecules do not form an exclusive list. As demonstrated herein, it has been determined that FGF21 and variants thereof can be of use when treating various metabolic conditions, such as Type I diabetes. Thus, any molecule that induces FGF21-like signaling can be employed in the disclosed methods. The terms “FGF21-like signaling” and “induces FGF21-like signaling,” when applied to molecules contemplated for use in the methods of the present disclosure, means that the molecule mimics, or modulates, an in vivo biological effect induced by the binding of (i) β-Klotho; (ii) FGFR1c, FGFR2c, FGFR3c or FGFR4; or (iii) a complex comprising β-Klotho and one of FGFR1c, FGFR2c, FGFR3c, and FGFR4 and induces a biological response that otherwise would result from FGF21 binding to (i) β-Klotho; (ii) FGFR1c, FGFR2c, FGFR3c or FGFR4; or (iii) a complex comprising β-Klotho and one of FGFR1c, FGFR2c, FGFR3c, and FGFR4 in vivo. In identifying molecules for use in the disclosed methods, a molecule is deemed to induce a biological response when the response is equal to or greater than 5%, and preferably equal to or greater than 10%, 15%, 20%, 25%, 30%, 35%, 40%, 45%, 50%, 55%, 60%, 65%, 70%, 75%, 80%, 85%, 90% or 95%, of the activity of a wild type FGF21 standard comprising the mature form of SEQ ID NO:4 or 8 (i.e., a mature form of the human FGF21 sequence) and has the following properties: exhibiting an efficacy level of equal to or more than 5% of an FGF21 standard (e.g., SEQ ID NOs:4 and 8), with an EC50 of equal to or less than 100 nM, e.g., 90 nM, 80 nM, 70 nM, 60 nM, 50 nM, 40 nM, 30 nM, 20 nM or 10 nM in (1) a recombinant FGF21 receptor mediated luciferase-reporter cell assay such as those described in WO 2011/071783; (2) ERK-phosphorylation in a recombinant FGF21 receptor mediated cell assay such as those described in WO 2011/071783; and (3) ERK-phosphorylation in human adipocytes as described in WO 2011/071783. The “potency” of a candidate molecule is defined as exhibiting an EC50 of equal to or less than 100 nM, e.g., 90 nM, 80 nM, 70 nM, 60 nM, 50 nM, 40 nM, 30 nM, 20 nM, 10 nM and preferably less than 10 nM of the molecule in the following assays: (1) the recombinant FGF21 receptor mediated luciferase-reporter cell assay described in WO 2011/071783; (2) the ERK-phosphorylation in the recombinant FGF21 receptor mediated cell assay described in WO 2011/071783; and (3) ERK-phosphorylation in human adipocytes as described in WO 2011/071783.

Accordingly, the disclosed methods can be performed using FGF21 mimetics, or molecules that mimic FGF21 activity but which themselves comprise a relatively low degree of sequence homology to a FGF21 polypeptide sequence (e.g., SEQ ID NO:4 or 8) or FGF21 variant sequence, or in some cases have no homology at all with FGF21. Such molecules are described in WO 2011/071783, WO 2011/068893, WO 2011/130417 and WO 2010/148142.

### VII. Pharmaceutical Compositions Comprising a FGF21 Polypeptide or Variant

Pharmaceutical compositions comprising a FGF21 polypeptide or FGF21 variant for use in the disclosed methods are provided. Such FGF21 polypeptide or FGF21 variant pharmaceutical compositions can comprise a therapeutically effective amount of a FGF21 polypeptide or FGF21 variant in admixture with a pharmaceutically or physiologically acceptable formulation agent selected for suitability with the mode of administration. A pharmaceutical composition suitable for use in the disclosed methods can comprise an FGF21 polypeptide or FGF21 variant disclosed herein.

The term “pharmaceutically acceptable carrier” or “physiologically acceptable carrier” as used herein refers to one or more formulation agents suitable for accomplishing or enhancing the delivery of a FGF21 polypeptide or FGF21 variant into the body of a human or non-human subject, and for use in the methods disclosed herein. The term includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In some cases it will be preferable to include isotonic agents, for example, sugars, polyalcohols such as mannitol, sorbitol, or sodium chloride in a pharmaceutical composition. Pharmaceutically acceptable substances such as wetting or minor amounts of auxiliary substances such as wetting or emulsifying agents, preservatives or buffers, which enhance the shelf life or effectiveness of the FGF21 polypeptide or FGF21 variant can also act as, or form a component of, a carrier. Acceptable pharmaceutically acceptable carriers are preferably nontoxic to recipients at the dosages and concentrations employed.

A pharmaceutical composition for use in the methods disclosed herein can contain formulation agent(s) for modifying, maintaining, or preserving, for example, the pH, osmolarity, viscosity, clarity, color, isotonicity, odor, sterility, stability, rate of dissolution or release, adsorption, or penetration of the composition. Suitable formulation agents include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as Polysorbate 20 or Polysorbate 80; Triton; tromethamine; lecithin; cholesterol or tyloxapal), stability enhancing agents (such as sucrose or sorbitol), tonicity enhancing agents (such as alkali metal halides—preferably sodium or potassium chloride—or mannitol sorbitol), delivery vehicles, diluents, excipients and/or pharmaceutical adjuvants (see, e.g., REMINGTON: THE SCIENCE AND PRACTICE OF PHARMACY, 19th edition, (1995); Berge et al., J. Pharm. Sci., 6661), 1-19 (1977). Additional relevant principles, methods, and agents are described in, e.g., Lieberman et al., PHARMACEUTICAL DOSAGE FORMS-DISPERSE SYSTEMS (2nd ed., vol. 3, 1998); Ansel et al., PHARMACEUTICAL DOSAGE FORMS & DRUG DELIVERY SYSTEMS (7th ed. 2000); Martindale, THE EXTRA PHARMACOPEIA (31st edition), Remington's PHARMACEUTICAL SCIENCES (16th-20^{th} and subsequent editions); The Pharmacological Basis Of Therapeutics, Goodman and Gilman, Eds. (9th ed.—1996); Wilson and Gisvolds' TEXTBOOK OF ORGANIC MEDICINAL AND PHARMACEUTICAL CHEMISTRY, Delgado and Remers, Eds. (10th ed., 1998); Principles of formulating pharmaceutically acceptable compositions also are described in, e.g., Aulton, PHARMACEUTICS: THE SCIENCE OF DOSAGE FORM DESIGN, Churchill Livingstone (New York) (1988), EXTEMPORANEOUS ORAL LIQUID DOSAGE PREPARATIONS, CSHP (1998), all of which references are incorporated herein by reference for any purpose).

The optimal pharmaceutical composition for use in the methods disclosed herein will be determined by a skilled artisan depending upon, for example, the intended route of administration, delivery format, and desired dosage (see, e.g., Remington's PHARMACEUTICAL SCIENCES, supra). Such compositions can influence the physical state, stability, rate of in vivo release, and rate of in vivo clearance of the a FGF21 polypeptide.

The primary vehicle or carrier in a pharmaceutical composition for use in the methods disclosed herein can be either aqueous or non-aqueous in nature. For example, a suitable vehicle or carrier for injection can be water, physiological saline solution, or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise a Tris buffer of about pH 7.0-8.5, or an acetate buffer of about pH 4.0-5.5, which can further include sorbitol or a suitable substitute. In one embodiment of the present invention, FGF21 polypeptide or FGF21 variant compositions can be prepared for storage by mixing the selected composition having the desired degree of purity with optional formulation agents (Remington's PHARMACEUTICAL SCIENCES, supra) in the form of a lyophilized cake or an aqueous solution. Furthermore, the FGF21 polypeptide product can be formulated as a lyophilizate using appropriate excipients such as sucrose.

The FGF21 polypeptide or FGF21 variant pharmaceutical compositions can be selected for parenteral delivery. Alternatively, the compositions can be selected for inhalation or for delivery through the digestive tract, such as orally.

The formulation components can be present in concentrations that are acceptable to the site of administration. For example, buffers can be used to maintain the composition at physiological pH or at a slightly lower pH, typically within a pH range of from about 5 to about 8.

When parenteral administration is contemplated, the therapeutic compositions for use in the disclosed methods can be in the form of a pyrogen-free, parenterally acceptable, aqueous solution comprising the desired FGF21 polypeptide in a pharmaceutically acceptable vehicle. A particularly suitable vehicle for parenteral injection is sterile distilled water in which a FGF21 polypeptide or FGF21 variant is formulated as a sterile, isotonic solution, properly preserved. Yet another preparation can involve the formulation of the desired molecule with an agent, such as injectable microspheres, bio-erodible particles, polymeric compounds (such as polylactic acid or polyglycolic acid), beads, or liposomes, that provides for the controlled or sustained release of the product which can then be delivered via a depot injection. Hyaluronic acid can also be used, and this can have the effect of promoting sustained duration in the circulation. Other suitable means for the introduction of the desired molecule include implantable drug delivery devices.

In one embodiment, a pharmaceutical composition can be formulated for inhalation. For example, a FGF21 polypeptide or FGF21 variant can be formulated as a dry powder for inhalation. FGF21 polypeptide or FGF21 variant inhalation solutions can also be formulated with a propellant for aerosol delivery. In yet another embodiment, solutions can be nebulized. Pulmonary administration is further described in International Publication No. WO 94/20069.

It is also contemplated that certain formulations can be administered orally within the context of the methods disclosed herein. In one embodiment of this method, FGF21 polypeptides or FGF21 variants that are administered in this fashion can be formulated with or without those carriers customarily used in the compounding of solid dosage forms such as tablets and capsules. For example, a capsule can be designed to release the active portion of the formulation at the point in the gastrointestinal tract when bioavailability is maximized and pre-systemic degradation is minimized. Additional agents can be included to facilitate absorption of the FGF21 polypeptide or FGF21 variant. Diluents, flavorings, low melting point waxes, vegetable oils, lubricants, suspending agents, tablet disintegrating agents, and binders can also be employed.

An alternative pharmaceutical composition can comprise an effective quantity of a FGF21 polypeptide or FGF21 variant in a mixture with non-toxic excipients that are suitable for the manufacture of tablets. By dissolving the tablets in sterile water, or another appropriate vehicle, solutions can be prepared in unit-dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional FGF21 polypeptide or FGF21 variant pharmaceutical compositions that can be of use in the methods disclosed herein will be evident to those skilled in the art, including formulations involving FGF21 polypeptides or FGF21 variants in sustained- or controlled-delivery formulations. Techniques for formulating a variety of other sustained- or controlled-delivery means, such as liposome carriers, bio-erodible microparticles or porous beads and depot injections, are also known to those skilled in the art (see, e.g., International Publication No. WO 93/15722, which describes the controlled release of porous polymeric microparticles for the delivery of pharmaceutical compositions, and Wischke & Schwendeman, (2008) Int. J. Pharm. 364:298-327, and Freiberg & Zhu, (2004) Int. J. Pharm. 282: 1-18, which discuss microsphere/microparticle preparation and use). As described herein, a hydrogel is an example of a sustained- or controlled-delivery formulation.

Additional examples of sustained-release preparations include semipermeable polymer matrices in the form of shaped articles, e.g. films, or microcapsules. Sustained release matrices can include polyesters, hydrogels, polylactides (U.S. Pat. No. 3,773,919 and European Patent No. 0 058 481), copolymers of L-glutamic acid and gamma ethyl-L-glutamate (Sidman et al., (1983) Biopolymers 22:547-56), poly(2-hydroxyethyl-methacrylate) (Langer et al., (1981) J. Biomed. Mater. Res. 15:167-277 and Langer, (1982) Chem. Tech. 12:98-105), ethylene vinyl acetate (Langer et al., supra) or poly-D(−)-3-hydroxybutyric acid (European Patent No. 0 133 988). Sustained-release compositions can also include liposomes, which can be prepared by any of several methods known in the art. See, e.g., Epstein et al., (1985) Proc. Natl. Acad. Sci. U.S.A. 82:3688-92; and European Patent Nos. 0 036 676, 0 088 046, and 0 143 949.

A pharmaceutical composition comprising a FGF21 polypeptide or FGF21 variant to be used for in vivo administration in the methods disclosed herein typically should be sterile. This can be accomplished by filtration through sterile filtration membranes. Where the composition is lyophilized, sterilization using this method can be conducted either prior to, or following, lyophilization and reconstitution. The composition for parenteral administration can be stored in lyophilized form or in a solution. In addition, parenteral compositions generally are placed into a container having a sterile access port, for example, an intravenous solution bag or vial having a stopper pierceable by a hypodermic injection needle.

Once the pharmaceutical composition has been formulated, it can be stored in sterile vials as a solution, suspension, gel, emulsion, solid, or as a dehydrated or lyophilized powder. Such formulations can be stored either in a ready-to-use form or in a form (e.g., lyophilized) requiring reconstitution prior to administration.

In a specific embodiment, the present invention is directed to kits for producing a single-dose administration unit. The kits can each contain both a first container having a dried protein and a second container having an aqueous formulation. Also disclosed are kits containing single and multi-chambered pre-filled syringes (e.g., liquid syringes and lyosyringes).

The effective amount of a pharmaceutical composition comprising a FGF21 polypeptide or FGF21 variant to be employed therapeutically in the methods disclosed herein will depend, for example, upon the therapeutic context and objectives. One skilled in the art will appreciate that the appropriate dosage levels for treatment will thus vary depending, in part, upon the molecule delivered, the indication for which a FGF21 polypeptide or FGF21 variant is being used, the route of administration, and the size (body weight, body surface, or organ size) and condition (the age and general health) of the patient. Accordingly, the clinician can titer the dosage and modify the route of administration to obtain the optimal therapeutic effect. A typical dosage can range from about 0.1 μg/kg to up to about 100 mg/kg or more, depending on the factors mentioned above.

The frequency of dosing employed in the methods disclosed herein will depend upon the pharmacokinetic parameters of the FGF21 polypeptide or FGF21 variant in the formulation being used. Typically, a clinician will administer the composition until a dosage is reached that achieves the desired effect. The composition can therefore be administered as a single dose, as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages can be ascertained through use of appropriate dose-response data, such as data obtained from a clinical trial involving the treatment of a metabolic disorder or condition, including Type 1 diabetes, with a FGF21 polypeptide or FGF21 variant.

The route of administration of the pharmaceutical composition is in accord with known methods, e.g., orally; through injection by intravenous, intraperitoneal, intracerebral (intraparenchymal), intracerebroventricular, intramuscular, intraocular, intraarterial, intraportal, or intralesional routes; by sustained release systems (which may also be injected); or by implantation devices. Where desired, the compositions can be administered by bolus injection or continuously by infusion, or by implantation device.

Alternatively or additionally, the composition can be administered locally via implantation of a membrane, sponge, or other appropriate material onto which the desired molecule has been absorbed or encapsulated. Where an implantation device is used, the device can be implanted into any suitable tissue or organ, and delivery of the desired molecule can be via diffusion, timed-release bolus, or continuous administration.

When practicing the disclosed methods, in order to deliver a drug, e.g., a FGF21 polypeptide or FGF21 variant, at a predetermined rate such that the drug concentration can be maintained at a desired therapeutically effective level over an extended period, a variety of different approaches can be employed. Such approaches can be useful when practicing the methods disclosed herein. In one example, a hydrogel comprising a polymer such as a gelatin (e.g., bovine gelatin, human gelatin, or gelatin from another source) or a naturally-occurring or a synthetically generated polymer can be employed. Any percentage of polymer (e.g., gelatin) can be employed in a hydrogel, such as 5, 10, 15 or 20%. The selection of an appropriate concentration can depend on a variety of factors, such as the therapeutic profile desired and the pharmacokinetic profile of the therapeutic molecule.

Examples of polymers that can be incorporated into a hydrogel include polyethylene glycol (“PEG”), polyethylene oxide, polyethylene oxide-co-polypropylene oxide, co-polyethylene oxide block or random copolymers, polyvinyl alcohol, poly(vinyl pyrrolidinone), poly(amino acids), dextran, heparin, polysaccharides, polyethers and the like.

Another factor that can be considered when generating a hydrogel formulation is the degree of crosslinking in the hydrogel and the crosslinking agent. In one embodiment, cross-linking can be achieved via a methacrylation reaction involving methacrylic anhydride. In some situations, a high degree of cross-linking may be desirable while in other situations a lower degree of crosslinking is preferred. In some cases a higher degree of crosslinking provides a longer sustained release. A higher degree of crosslinking may provide a firmer hydrogel and a longer period over which drug is delivered.

Any ratio of polymer to crosslinking agent (e.g., methacrylic anhydride) can be employed to generate a hydrogel with desired properties. For example, the ratio of polymer to crosslinker can be, e.g., 8:1, 16:1, 24:1, or 32:1. For example, when the hydrogel polymer is gelatin and the crosslinker is methacrylate, ratios of 8:1, 16:1, 24:1, or 32:1 methyacrylic anhydride:gelatin can be employed.

### VIII. Methods of Treating Metabolic Condition or Disorder Using the Disclosed FGF21 Polypeptides and FGF21 Variants and Nucleic Acids

FGF21 polypeptides and FGF21 variants can be used to treat, diagnose or ameliorate, a metabolic condition or disorder when employed in the methods disclosed herein. In one embodiment, the metabolic disorder to be treated is diabetes, e.g., type 1 diabetes. In another embodiment, the metabolic condition or disorder is obesity. In other embodiments the metabolic condition or disorder is dyslipidemia, elevated glucose levels, elevated insulin levels or diabetic nephropathy. The FGF21 polypeptides can be provided to a subject in the form of a pharmaceutical composition.

In one example, a metabolic condition or disorder that can be treated or ameliorated using a FGF21 polypeptide or FGF21 variant is a state in which a human subject has a fasting blood glucose level of 125 mg/dL or greater, for example 130, 135, 140, 145, 150, 155, 160, 165, 170, 175, 180, 185, 190, 195, 200 or greater than 200 mg/dL. In one embodiment of the disclosed methods achieving a fasting blood glucose level of 70-100 mg/dL can be a target goal, e.g., administering enough FGF21 polypeptide or variant to a human patient in order to achieve a fasting blood glucose level of 70, 75, 80, 85, 90, 95 or 100 mg/dL. Measurements of fasting glucose level can be obtained using any of a variety of well-known methods or apparatus. For example, in one embodiment an Olympus AU400e Chemistry Analyzer (Olympus America, Inc., Center Valley, Pa.) can be employed.

Blood glucose levels can be determined in the fed or fasted state, or at random. In another embodiment a metabolic condition or disorder that can be treated or ameliorated using a FGF21 polypeptide or FGF21 variant is a state in which a human subject has a fed (not postpriandial) blood glucose level of greater than 120 mg/dL. For the fed (not postprandial) state, the disclosed methods can be employed to achieve a target blood glucose level in a human patient, such as 80-120 mg/dL. e.g., 80, 85, 90, 95, 100, 105, 110, 115 or 120 mg/dL. Measurements of blood glucose level in the fed (not postprandial) state can be obtained using any of a variety of well-known methods or apparatus. For example, in one embodiment an Olympus AU400e Chemistry Analyzer (Olympus America, Inc., Center Valley, Pa.) can be employed.

In another embodiment a metabolic condition or disorder that can be treated or ameliorated using a FGF21 polypeptide or FGF21 variant is a state in which a human subject has a fasting triglyceride level of greater than 150 mg/dL. One exemplary target fasting triglyceride level is less than 150 mg/dL and an exemplary method comprises administering enough FGF21 polypeptide or variant to a human patient in order to achieve a fasting triglyceride level of 150, 145, 140, 135, 130, 125, 120, 115, 110, 105, 100, 95, 90, 85, 80, 75, 70, 65, 60, 55, 50, 45, 40, 35, 30, 25, 20 or 10 mg/dL. Measurements of fasting triglyceride level can be obtained using any of a variety of well-known methods or apparatus. For example, in one embodiment an Olympus AU400e Chemistry Analyzer (Olympus America, Inc., Center Valley, Pa.) can be employed.

In another embodiment a metabolic condition or disorder that can be treated or ameliorated using a FGF21 polypeptide or FGF21 variant is a state in which a human subject has a fasting total cholesterol level of greater than 200 mg/dL. One exemplary target total cholesterol level is less than 200 mg/dL and an exemplary method comprises administering enough FGF21 polypeptide or variant to a human patient in order to achieve a fasting total cholesterol level of 200, 195, 190, 185, 180, 175, 170, 165, 160, 155, 150, 145, 140, 135, 130, 125, 120, 115, 110, 105, 100, 95, 90, 85, 80, 75, 70, 65, 60, 55, 50, 45, 40, 35, 30, 25, 20 or 10 mg/dL. Measurements of fasting total cholesterol level can be obtained using any of a variety of well-known methods or apparatus. For example, in one embodiment an Olympus AU400e Chemistry Analyzer (Olympus America, Inc., Center Valley, Pa.) can be employed.

In another embodiment a metabolic condition or disorder that can be treated or ameliorated using a FGF21 polypeptide or FGF21 variant is a state in which a human subject has a blood glucose level of greater than 140 mg/dL two hours after administration of glucose (i.e., an oral glucose tolerance test, “OGTT”). For an OGTT, one exemplary target plasma glucose level is less than 140 mg/dL and an exemplary method comprises administering enough FGF21 polypeptide or variant to a human patient in order to achieve a plasma glucose level 2 hours after administration of glucose to a human patient of 140, 135, 130, 125, 120, 115, 110, 105, 100, 95, 90, 85, 80, 75, 70, 65, 60, 55 or 50 mg/dL. Measurements of plasma glucose level can be obtained using any of a variety of well-known methods or apparatus. For example, in one embodiment an Olympus AU400e Chemistry Analyzer (Olympus America, Inc., Center Valley, Pa.) can be employed.

In another embodiment a metabolic condition or disorder that can be treated or ameliorated using a FGF21 polypeptide or FGF21 variant is a state in which a human subject has an insulin level that is not deemed physiologically advisable as determined by a trained clinician or physician. Insulin levels can be obtained using any of a variety of well-known methods or apparatus. For example, in one embodiment a Human Multiplex Endocrine Kit (HENDO-75K, Millipore Corp., Billerica, Mass.) can be employed.

In another embodiment a metabolic condition or disorder that can be treated or ameliorated using a FGF21 polypeptide or FGF21 variant is a state in which a human subject has a Body Mass Index (“BMI”) of greater than 25 kg/m². One exemplary BMI within the range of 18.5-25 kg/m² and an exemplary method comprises administering enough FGF21 polypeptide or variant to a human patient in order to achieve a BMI of 18.5, 19.0, 19.5, 20.0, 20.5, 21.0, 21.5, 22.0, 22.5, 23.0, 23.5, 24.0, 24.5 or 25.0 kg/m². Measurements of BMI can be obtained by determining a patient's weight and height.

In various embodiments, a subject is a human having a blood glucose level of 100 mg/dL or greater can be treated with a FGF21 polypeptide or FGF21 variant.

The metabolic condition or disorder that can be treated or ameliorated using a FGF21 polypeptide or FGF21 variant can also comprise a condition in which a subject is at increased risk of developing a metabolic condition. For a human subject, such conditions include a fasting blood glucose level of about 100 mg/dL. Conditions that can be treated using a pharmaceutical composition comprising a FGF21 polypeptide or FGF21 variant can also be found in the American Diabetes Association Standards of Medical Care in Diabetes Care-2011, American Diabetes Association, Diabetes Care Vol. 34, No. Supplement 1, S11-S61, 2010, incorporated herein by reference.

In application, a metabolic disorder or condition, such as Type 1 diabetes, elevated fasting glucose levels, elevated insulin levels, dyslipidemia, obesity, elevated fed plasma glucose levels, elevated fasting triglyceride levels, elevated fasting total cholesterol levels elevated plasma glucose levels following an OGTT, and complications of diabetes, such as nephropathy, neuropathy, retinopathy, ischemic heart disease, peripheral vascular disease and cerebrovascular disease can be treated by administering a therapeutically effective dose of a FGF21 polypeptide, e.g., a human FGF21 polypeptide such as those of SEQ ID NOs:2, 4, 6 or 8, or an FGF21 variant provided herein, such as a variant described in Tables 1-13 and those recited in the Sequence Listing associated with the instant disclosure, to a patient in need thereof. The administration can be performed as described herein, such as by IV injection, intraperitoneal (IP) injection, subcutaneous injection, intramuscular injection, or orally in the form of a tablet or liquid formation. In some situations, a therapeutically effective or preferred dose of a FGF21 polypeptide or FGF21 variant can be determined by a clinician. A therapeutically effective dose of FGF21 polypeptide or FGF21 variant will depend, inter alia, upon the administration schedule, the unit dose of agent administered, whether the FGF21 polypeptide or FGF21 variant is administered in combination with other therapeutic agents, the immune status and the health of the recipient. The term “therapeutically effective dose,” as used herein, means an amount of FGF21 polypeptide or FGF21 variant that elicits a biological or medicinal response in a tissue system, animal, or human being sought by a researcher, medical doctor, or other clinician, which includes alleviation or amelioration of the symptoms of the disease or disorder being treated, i.e., an amount of a FGF21 polypeptide or FGF21 variant that supports an observable level of one or more desired biological or medicinal response, for example lowering blood glucose, insulin, triglyceride, or cholesterol levels; reducing body weight; or improving glucose tolerance, energy expenditure, or insulin sensitivity.

It is noted that a therapeutically effective dose of a FGF21 polypeptide or FGF21 variant can also vary with the desired result. Thus, for example, in situations in which a lower level of blood glucose is indicated a dose of a FGF21 polypeptide or FGF21 variant will be correspondingly higher than a dose in which a comparatively lower level of blood glucose is desired. Conversely, in situations in which a higher level of blood glucose is indicated a dose of a FGF21 polypeptide or FGF21 variant will be correspondingly lower than a dose in which a comparatively higher level of blood glucose is desired.

In one embodiment, a method of the instant disclosure comprises first measuring a baseline level of one or more metabolically-relevant compounds such as glucose, insulin, cholesterol, lipid in a subject. A pharmaceutical composition comprising a FGF21 polypeptide or FGF21 variant is then administered to the subject. After a desired period of time, the level of the one or more metabolically-relevant biomarkers or compounds (e.g., blood glucose, insulin, cholesterol and/or lipid levels) in the subject is again measured. The two levels can then be compared in order to determine the relative change in the metabolically-relevant compound in the subject. Depending on the conclusions of that comparison, another dose of the pharmaceutical composition comprising a FGF21 polypeptide or FGF21 variant can be administered to achieve a desired level of one or more metabolically-relevant compound. Again, the levels of relevant biomarkers or compounds can be assessed and a determination made as to the next step in the subject's therapeutic regimen (e.g., one or more further administrations or the pharmaceutical composition, another form of therapy, a combination of the pharmaceutical composition with another therapeutic molecule, etc).

It is noted that in various embodiments of the disclosed methods a pharmaceutical composition comprising a FGF21 polypeptide or FGF21 variant can be co-administered with another compound. The identity and properties of compound co-administered with the FGF21 polypeptide or FGF21 variant will depend on the nature of the condition to be treated or ameliorated. A non-limiting list of examples of compounds that can be administered in combination with a pharmaceutical composition comprising a FGF21 polypeptide or FGF21 variant include rosiglitizone, pioglitizone, repaglinide, nateglitinide, metformin, exenatide, stiagliptin, pramlintide, glipizide, glimeprirideacarbose, and miglitol.

### IX. Kits

Also provided are kits for practicing the disclosed methods. Such kits can comprise a pharmaceutical composition such as those FGF21 polypeptides and FGF21 variants described herein, including nucleic acids encoding the peptides or proteins provided herein, vectors and cells comprising such nucleic acids, and pharmaceutical compositions comprising such nucleic acid-containing compounds, which can be provided in a sterile container. Optionally, instructions on how to employ the provided pharmaceutical composition in the treatment of a metabolic disorder can also be included or be made available to a patient or a medical service provider.

In one aspect, a kit comprises (a) a pharmaceutical composition comprising a therapeutically effective amount of an FGF21 polypeptide or FGF21 variant; and (b) one or more containers for sterilely storing the pharmaceutical composition. Such a kit can also comprise instructions for the use thereof; the instructions can be tailored to the precise metabolic disorder being treated. The instructions can describe the use and nature of the materials provided in the kit. In certain embodiments, kits include instructions for a patient to carry out administration to treat a metabolic disorder, such as elevated glucose levels, elevated insulin levels, obesity, type 1 diabetes, dyslipidemia, diabetic nephropathy and complications of diabetes, such as nephropathy, neuropathy, retinopathy, ischemic heart disease, peripheral vascular disease and cerebrovascular disease.

Instructions can be printed on a substrate, such as paper or plastic, etc, and can be present in the kits as a package insert, in the labeling of the container of the kit or components thereof (e.g., associated with the packaging), etc. In other embodiments, the instructions are present as an electronic storage data file present on a suitable computer readable storage medium, e.g. a CD-ROM, diskette, etc. In yet other embodiments, the actual instructions are not present in the kit, but means for obtaining the instructions from a remote source, such as over the internet, are provided. An example of this embodiment is a kit that includes a web address where the instructions can be viewed and/or from which the instructions can be downloaded.

Often it will be desirable that some or all components of a kit are packaged in suitable packaging to maintain sterility. The components of a kit can be packaged in a kit containment element to make a single, easily handled unit, where the kit containment element, e.g., box or analogous structure, may or may not be an airtight container, e.g., to further preserve the sterility of some or all of the components of the kit.

Throughout the instant disclosure references to published documents have been provided. All documents recited in the instant disclosure are incorporated by reference herein in their entireties and for any purpose.

### EXAMPLES

The following examples, including the experiments conducted and results achieved, are provided for illustrative purposes only and are not to be construed as limiting the present invention.

### Introduction

Previous pharmacological studies with recombinant FGF21 have demonstrated its potent glucose-lowering effects in a variety of type 2 diabetic rodent and primate models. The metabolic actions of FGF21 have been well-established in these insulin resistant models and highlight its potential as a therapeutic for non insulin dependent diabetes mellitus (NIDDM). However, no studies have thus far been documented to examine the glucose lowering potential of FGF21 in type 1 diabetes, also referred to as insulin dependent diabetes mellitus (IDDM). The following Examples demonstrate various therapeutically-relevant effects, including glucose lowering and beta cell protective effects, of FGF21 when administered to a type 1 diabetes rodent model.

### Example 1

### Effect of Human FGF21 on High-Dose Streptozotocin (STZ)-Induced Type 1 Diabetic Mice

This study was conducted to evaluate the glucose-lowering and other metabolic effect of human FGF21 (SEQ ID NO:4), human insulin and their combination in STZ-induced type 1 diabetic mice.

Male C57BL6 mice were obtained from Harlan Laboratories and delivered at 7 weeks of age. Upon arrival, mice were single-housed and maintained in controlled environmental conditions with 12 hour light (6:30 AM-6:30 PM) and dark cycles (6:30 PM-6:30 AM). Mice were fed a standard rodent chow diet (2020× Harlan Teklad) with free-access to drinking water.

Following one week of acclimation, plasma glucose and/or body weight measurements were made. Mice were subsequently fasted for four hours by placing them into a fresh cage without chow. Mice were allowed free-access to drinking water. A single intraperitoneal (IP) injection of STZ (Streptozotocin, Sigma S-1030) at 180 mg/kg was administered into these mice to impair insulin producing beta cells within the pancreas and induce an insulin deficient type 1 diabetes-like phenotype. Rodent chow was then placed back into cages and mice were maintained on 10% sucrose water for 48 hours to prevent acute hypoglycemia. Regular drinking water was given 48 hours later. Daily morning body weights were measured during the induction process. At 72 hours post STZ injection (Day j), body weight and plasma glucose levels were measured and blood samples were collected from all mice. Mice demonstrating body weight loss from 1.2 g to 4.3 g and plasma glucose levels >410 mg/dL were selected for the study. Mice were then assigned into vehicle or treatment groups using plasma glucose and body weight values as randomization criteria. Vehicle (10 mM KPO₄, 5% Sorbitol, pH8.0), insulin (Humulin R, 5 IU/kg), recombinant human FGF21 (1 mg/kg), or both insulin (Humulin R, 5 IU/kg) and recombinant human FGF21 (1 mg/kg) were injected IP into mice twice daily at indicated doses. Blood glucose was measured on Day 3 after treatment initiation, at 1 hour and 4 hours after the morning injection and on Day 5, at 1 hour after the morning injection. On Day 5, after the 1 hour blood glucose measurement, mice were euthanized and terminal blood samples were collected. Body weight was measured daily during the study period.

Plasma was prepared from blood samples collected at baseline and terminal for clinical chemistry and endocrine hormone analysis. Clinical chemistry parameters, including plasma glucose, total cholesterol, triglycerides, and non-esterified fatty acids (NEFA), were measured using the Olympus AU400e Chemistry Analyzer (Olympus America, Inc; Center Valley, Pa.). Insulin and glucagon levels were determined by a multiplex murine endocrine kit (MENDO-75K, Millipore Corp., Billerica, Mass.).

### Effect of FGF21 on Plasma Glucose:

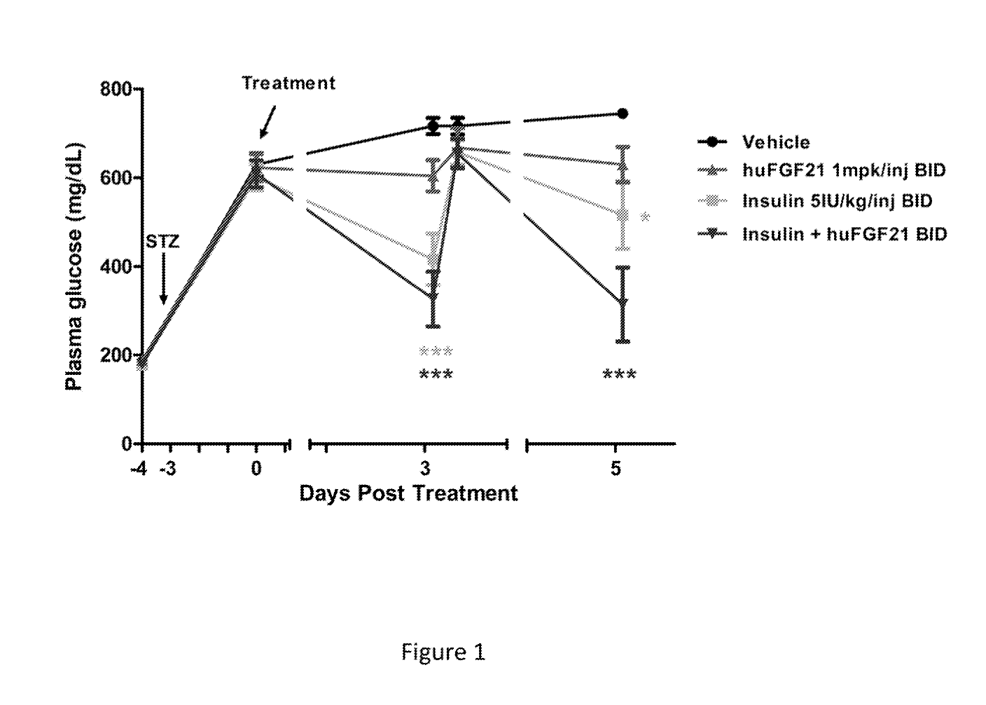

As shown in FIG. 1, following STZ injection, the blood glucose levels in all groups increased from normal to mean levels ranging from 601 to 630 mg/dL. The blood glucose levels continued to increase in vehicle group to >700 mg/dL during the five day treatment period. Treatment with recombinant human FGF21 (1 mg/kg) or insulin (5 IU/kg) reduced blood glucose levels by 16% and 42%, respectively, on Day 3. An additive 54% reduction of blood glucose levels was observed in FGF21 and insulin combination group. Blood glucose levels returned to baseline 4 hours post injection of all compounds. On day 5, similar findings were observed. Blood glucose level reductions for recombinant human FGF21, insulin, and combination treatment were 15%, 31%, and 58%, respectively, relative to vehicle group.

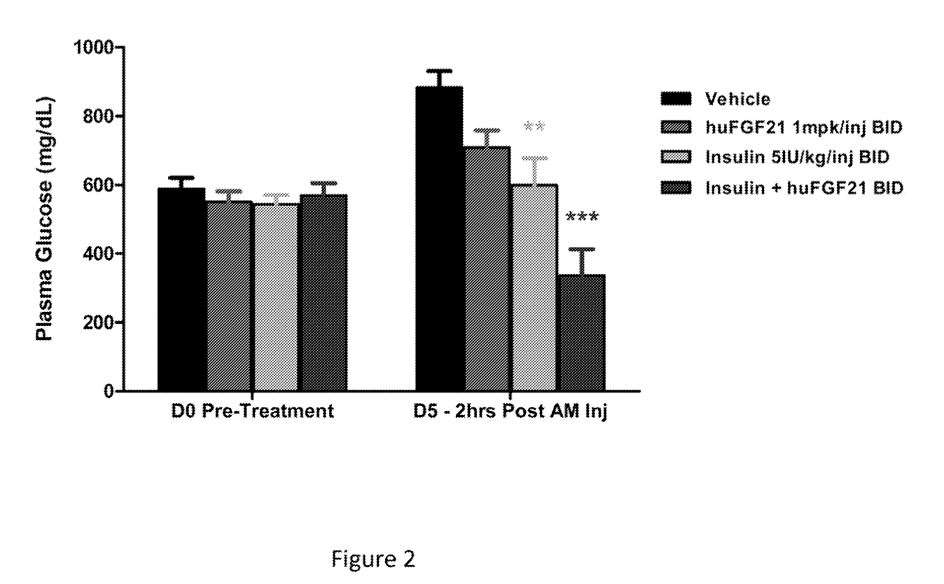

Plasma from blood samples collected at baseline (day 0) and approximately 2 hours post the morning injection on day 5 was run on a clinical chemistry analyzer for a more precise plasma glucose measurement. The results are shown in FIG. 2. Similar to the blood glucose measurements obtained from the glucometer shown in FIG. 1, human FGF21, insulin, and combination treatment resulted in 20%, 32%, and 62% glucose level reductions relative to vehicle group, respectively.

### Effect of FGF21 on Lipid Levels:

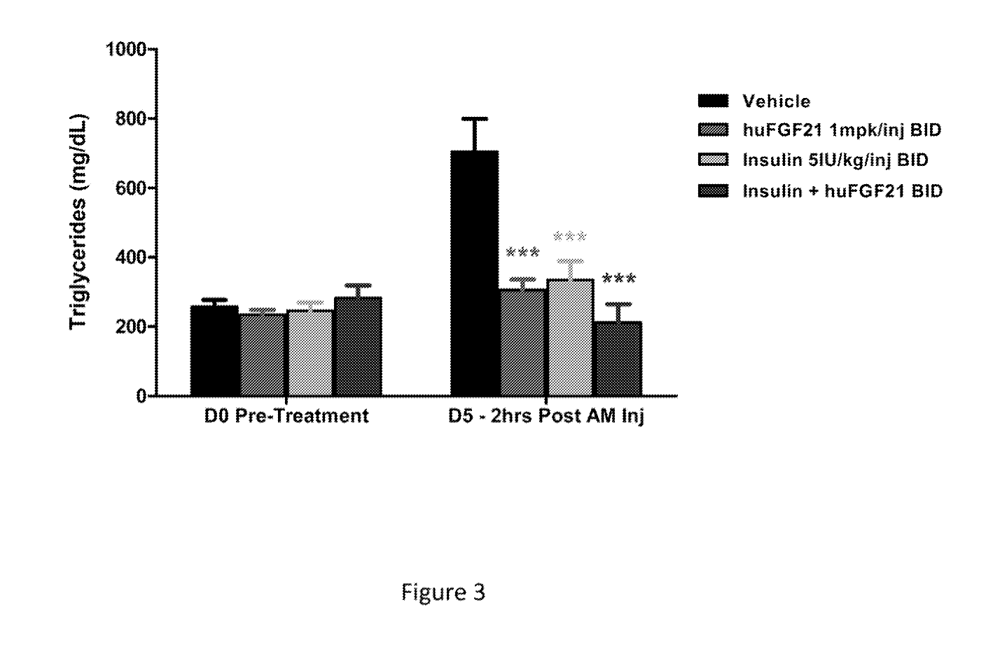

Blood samples collected at baseline (Day 0) and approximately two hours post the morning injection on Day 5 were run on a clinical chemistry analyzer to measure plasma lipid levels. From Day 0 to Day 5, plasma triglyceride, total cholesterol, and NEFA levels in vehicle treated mice increased 2-3 folds as type 1 diabetes progressively worsened. Human FGF21 (1 mg/kg) treatment alone lowered plasma triglyceride levels, similar to levels observed in insulin (Humulin R, 5 IU/kg) treated animals (FIG. 3). A further plasma triglyceride lowering effect was observed for the combination treatment group. Relative to vehicle group, plasma triglyceride level reductions for human FGF21, insulin, and combination treatment, were 57%, 53%, and 70%, respectively (FIG. 3).

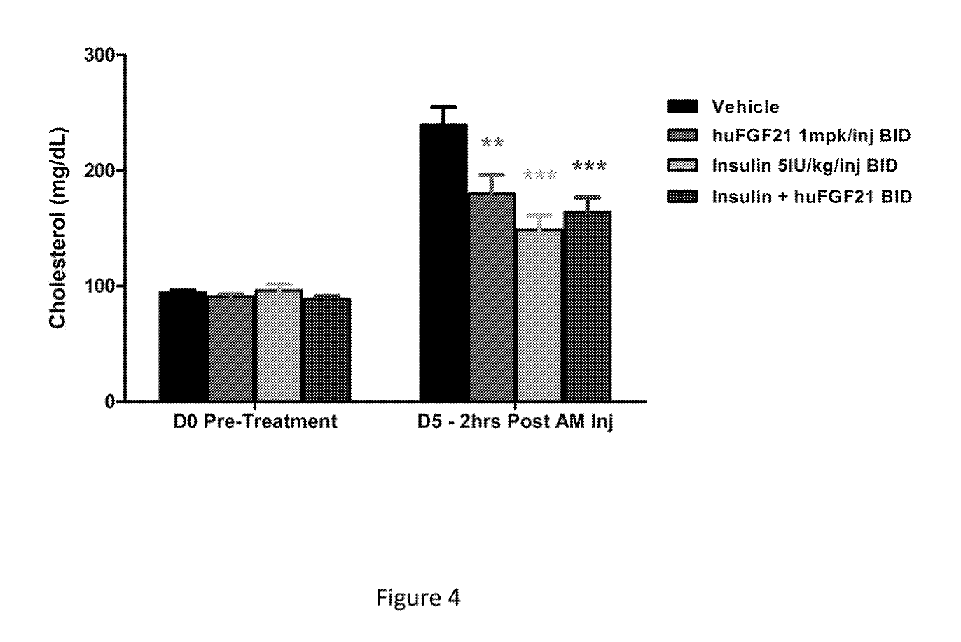

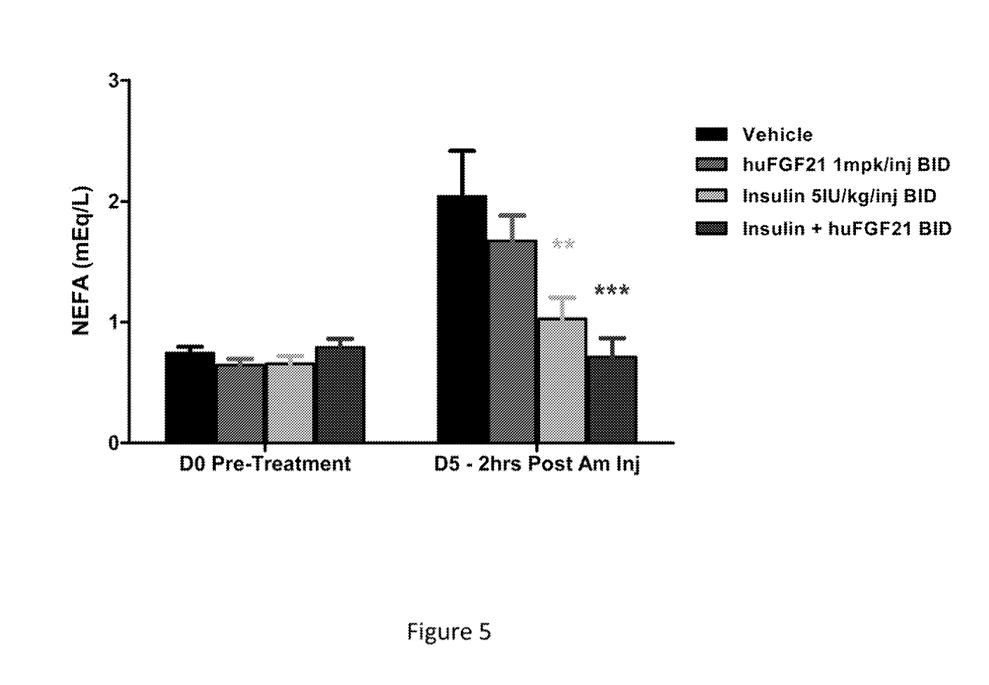

Treatment with FGF21 also lowered total cholesterol and NEFA levels. Relative to vehicle group, total cholesterol level reductions for human FGF21, insulin, and combination treatment, were 57%, 53%, and 70%, respectively (FIG. 4). NEFA levels were reduced in human FGF21, insulin, and combination treated mice by 18%, 50%, and 65% relative to vehicle treated mice, respectively (FIG. 5).

### Effect of FGF21 on Insulin Levels:

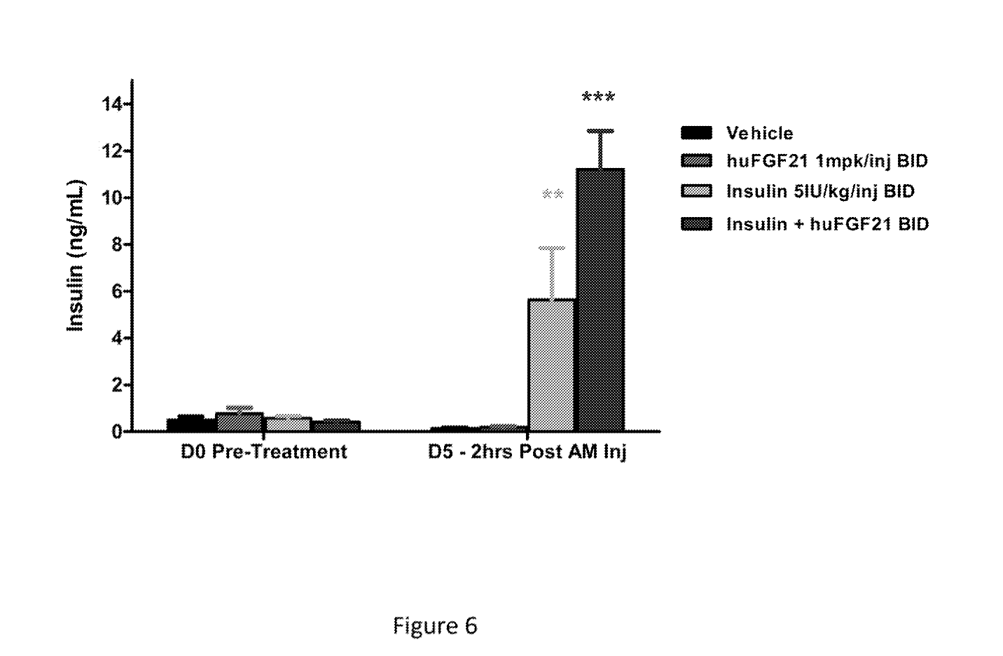

Insulin levels were evaluated in STZ-treated mice injected twice daily with vehicle, recombinant human FGF21 (1 mg/kg), insulin (Humulin R, 5 IU/kg), or combination of insulin (Humulin R, 5 IU/kg) and FGF21 (1 mg/kg). Treatment with FGF21 alone didn't restore plasma insulin level in STZ-treated mice (FIG. 6). However, plasma insulin levels were higher in FGF21 and insulin combination treatment group than in insulin treatment alone group, suggests a possible insulin stabilization effect of FGF21 (FIG. 6).

### Effect of FGF21 on Glucagon Levels:

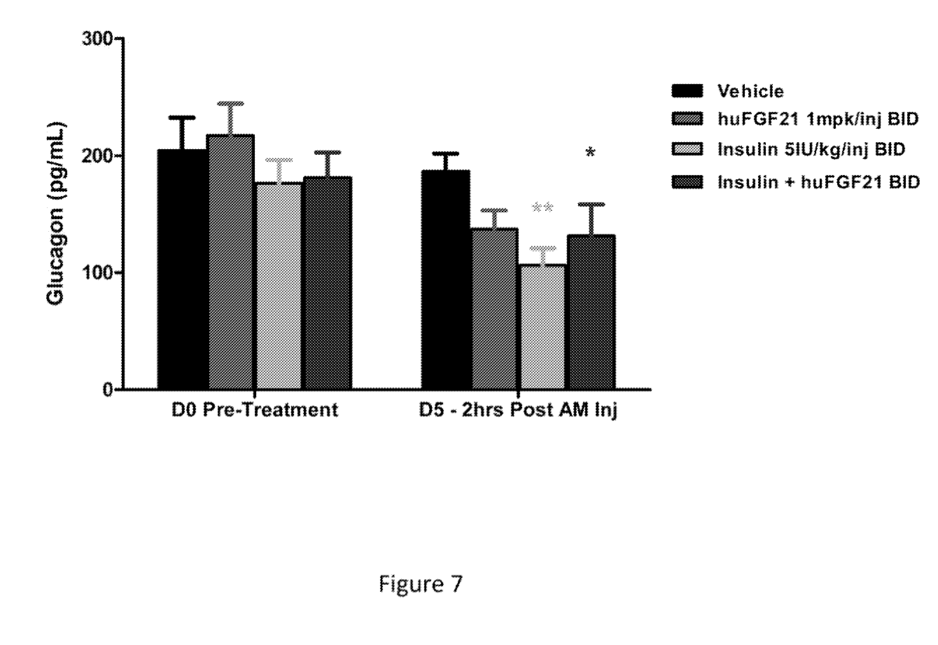

FIG. 7 demonstrates the ability of FGF21 to lower plasma glucagon levels in a STZ-induced type 1 diabetic rodent model. Lower glucagon levels were present in all treatment groups as compared to vehicle group. Recombinant human FGF21 (1 mg/kg), insulin (Humulin R, 5 IU/kg), or combination of insulin (Humulin R, 5 IU/kg) and recombinant human FGF21 (1 mg/kg) treatment reduced glucagon levels by 27%, 43%, and 30% relative to vehicle treatment, respectively.

### Example 2

### Effect of the Dual-PEGylated Human FGF21 Variant (E37C, R77C, P171G) on High-Dose STZ-Induced Type 1 Diabetic Mice

In Example 1, it was demonstrated that native human FGF21 treatment is capable of lowering plasma glucose levels in a STZ-induced type 1 diabetic rodent model. However, this effect is short-lived, as plasma glucose levels return within four hours post injection (FIG. 1). In order to evaluate the plasma glucose lowering effects over a prolonged timeframe, two polyethylene glycol (PEG) molecules (20 kD) were chemically fused at positions 37 and 77, to a human FGF21 variant (E37C, R77C, P171G; positions of the mutations are relative to SEQ ID NO:4). This dual-PEGylated human FGF21 variant has been demonstrated to exhibit superior glucose-lowering efficacy to native human FGF21 in previous rodent studies, possibly as a result of improved pharmacokinetics. The current study was conducted to evaluate whether this dual-PEGylated human FGF21 variant could produce a sustained glucose-lowering effect in STZ-induced type 1 diabetic mice following a single administration.

The high-dose STZ (180 mg/kg)-induced type 1 diabetic mouse model was generated as described in Example 1. At 72 hours post STZ injection (Day 0), mice demonstrating body weight loss from 1.2 g to 3.3 g and plasma glucose levels in the range of 367 to 652 mg/dL were selected for the study and assigned into vehicle or respective treatment groups. A single IP injection of vehicle (10 mM Tris-HCl, 150 mM NaCl, pH 8.5) or dual-PEGylated human FGF21 variant (E37C, R77C, P171G) at 1 and 5 mg/kg was administered into mice. Blood glucose was measured on Days 1, 3, 5, and 7, following administration of vehicle or dual-PEGylated human FGF21 variant (E37C, R77C, P171G). Body weight was measured daily during the entire study period.

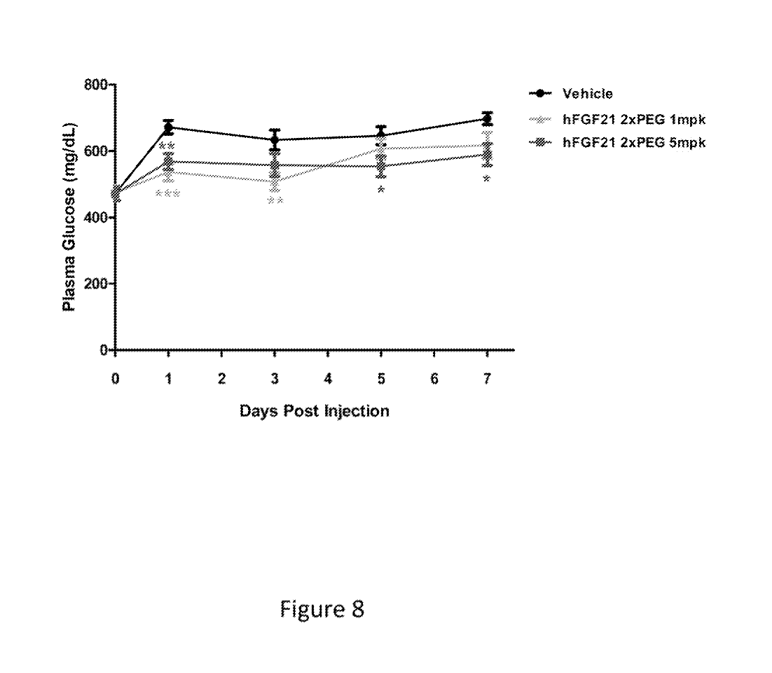

As shown in FIG. 8, by Day 1, plasma glucose levels in dual-PEGylated human FGF21 variant (E37C, R77C, P171G) (1 and 5 mg/kg) were reduced by 20% and 15% relative to vehicle levels, respectively. These levels were maintained for both dose groups at day 3 with glucose reductions by 20% and 12% relative to vehicle, respectively. The high dose dual-PEGylated human FGF21 variant (5 mg/kg) continued to show efficacy at Day 5 and Day 7 with plasma glucose reduction by about 15% relative to vehicle. The efficacy diminished for the lower dose dual-PEGylated human FGF21 variant (1 mg/kg) by day 5.

### Example 3

### Effect of the Dual-PEGylated Human FGF21 Variant (E37C, R77C, P171G) in Multiple Low Dose STZ-Induced Type 1 Diabetic Mice (Prevention)

A multiple low dose (MLD) STZ-induced type 1 diabetic mouse model was generated. The MLD-STZ model more closely mimics type 1 diabetes development in humans than the single high dose STZ model mentioned in the previous studies. The MLD method causes gradual loss of beta cells of the pancreas as each successive low dose STZ injection. This generates an initial inflammatory response towards the beta cells of the pancreas. Over the course of 2-3 weeks, this innate immunological response increases and destroys the insulin producing beta cells of the pancreas leading to T1DM. In contrast, the single high dose STZ (180 mg/kg) method rapidly destroys beta cells in the pancreas with the first 24 to 48 hours following STZ injection. Although both methods ultimately result in insulin deficient type 1 diabetic mice, the MLD method is predominantly driven by an immunological response, whereas the single high dose method is largely driven by the toxic effects of STZ. In this study, we evaluated the effects of the dual-PEGylated human FGF21 variant (E37C, R77C, P171G) on T1DM progression in MLD STZ-induced mice.

Male C57BL6 mice were obtained from Harlan Laboratories and delivered at 7 weeks of age. Upon arrival, mice were single-housed and maintained in controlled environmental conditions. Following 5 days of acclimation, mice above 20 g of body weight were administered five consecutive daily intraperitoneal (IP) injections of STZ (Streptozotocin, Sigma S-1030) at 40 mg/kg/day. Mice were fasted for four hours before receiving STZ injection each day. Daily morning body weights were monitored during the induction process. At 72 hours post the fifth STZ injection (Day 0), body weight and plasma glucose measurements were made and plasma was collected from all mice. Mice with plasma glucose values >200 mg/dL were then randomly assigned into vehicle or treatment group, based on plasma glucose and body weight as sorting criteria.

Vehicle (10 mM Tris-HCl, 150 mM NaCl, pH 8.5) or dual-PEGylated 20 kd human FGF21 variant (E37C, R77C, P171G)-(1 mg/kg) was injected IP into mice every four days, beginning on Day 0. Blood glucose was measured on day 2, 6, 10, 14, 18 and 22 after treatment initiation. On Day 27, seven days post the last injection, mice were euthanized and terminal blood samples were collected. Body weight was measured every other day during the study period.

### Effect of the Dual-PEGylated Human FGF21 Variant (E37C, R77C, P171G) on Plasma Glucose:

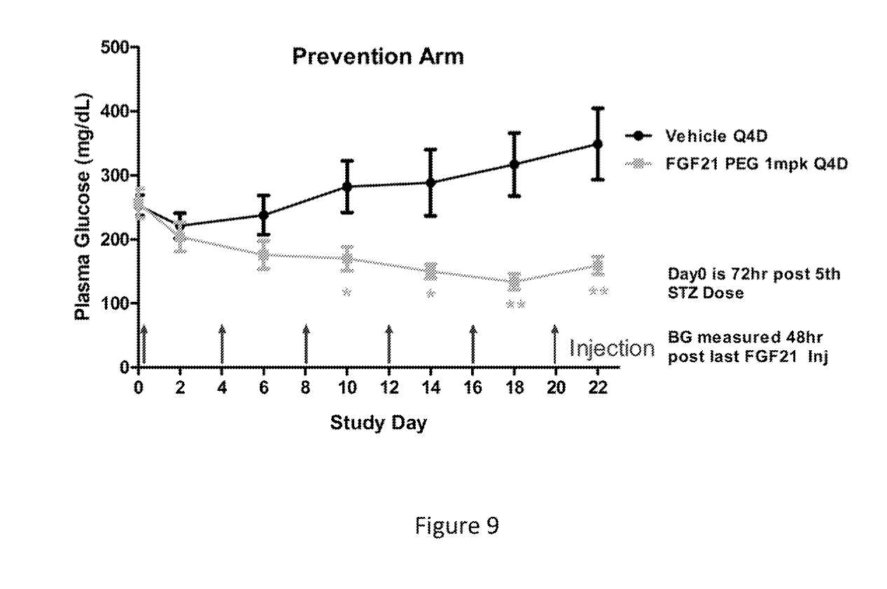

At 72 hours post the fifth STZ injection (day 0), the baseline mean blood glucose for vehicle and treatment groups was 253 mg/dL and 256 mg/dL, respectively. The plasma glucose level increased over the course of the study in vehicle group, while it was reduced in the dual-PEGylated human FGF21 variant (E37C, R77C, P171G) group by 8% (Day 2), 26% (Day 6), 40% (Day 10), 48% (Day 14), 58% (Day 18), and 54% (Day 22), relative to vehicle (FIG. 9).

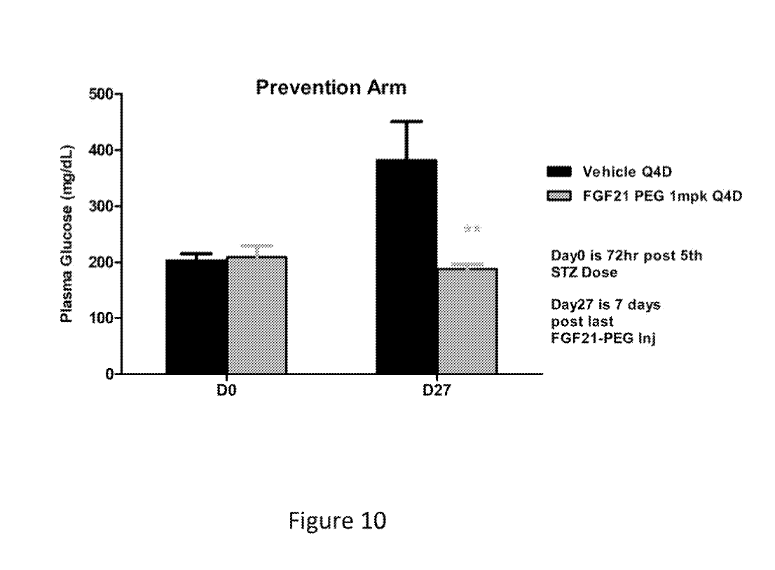

Plasma from blood samples collected on Day 0 and Day 27 (seven days post the last injection) were run on a clinical chemistry analyzer for a more precise plasma glucose measurement. Similar to blood glucose reductions shown in FIG. 9, treatment with the dual-PEGylated human FGF21 variant (E37C, R77C, P171G) prevented plasma glucose elevation and reduced plasma glucose levels to normoglycemic levels (FIG. 10). Relative to vehicle group, plasma glucose reduction for the dual-PEGylated human FGF21 variant (E37C, R77C, P171G) was 51%.

### Effect of the Dual-PEGylated Human FGF21 Variant (E37C, R77C, P171G) on Lipid Levels:

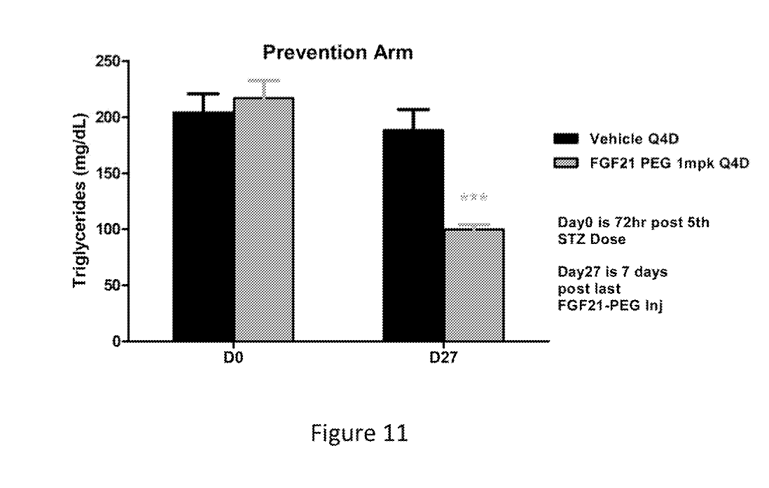

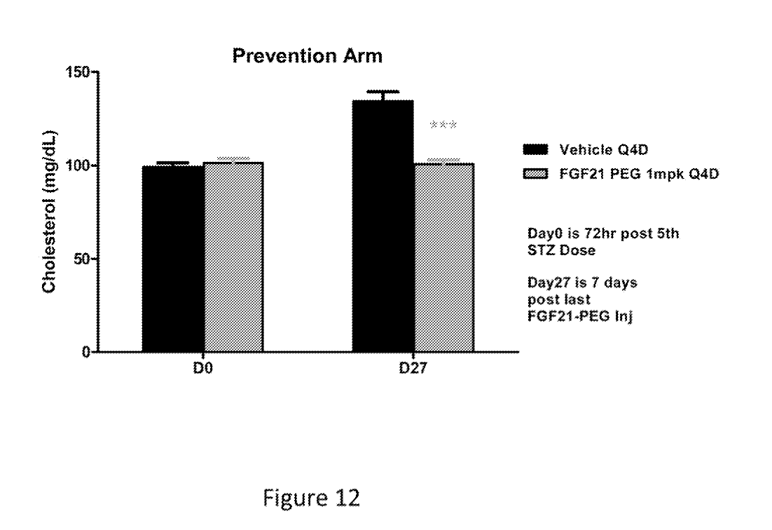

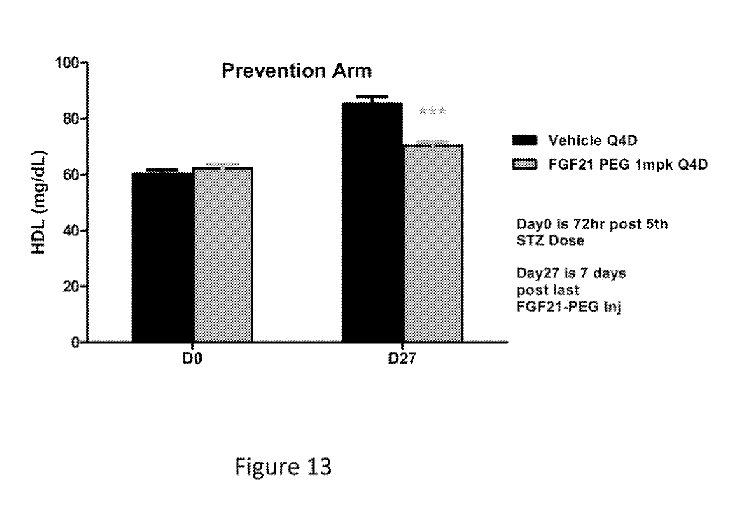

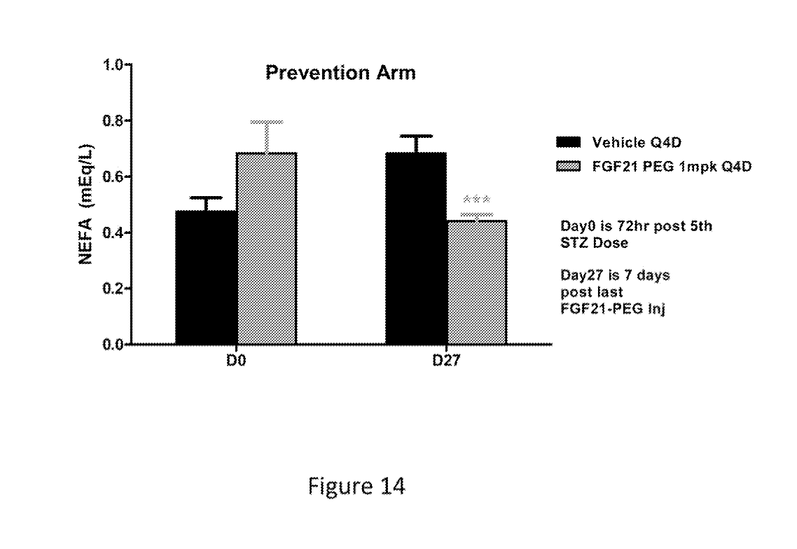

From Day 0 to Day 27, plasma triglyceride levels in vehicle treated mice remained stable, while reduced in mice treated with the dual-PEGylated human FGF21 variant (E37C, R77C, P171G)mice by 47% (FIG. 11). Treatment with FGF21 also lowered total cholesterol by 25% (FIG. 12), HDL-cholesterol by 18% (FIG. 13), and NEFA levels by 35% (FIG. 14), relative to vehicle group, respectively.

### Effect of the Dual-PEGylated Human FGF21 Variant on Insulin and Glucagon Levels:

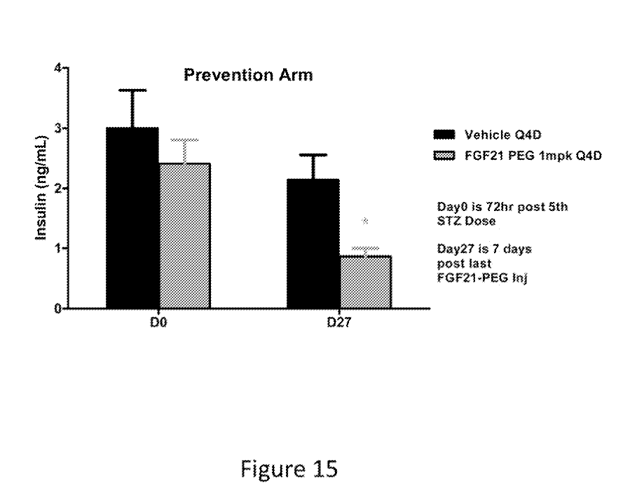

In comparison to the single high dose of STZ model, it is evident that the MLD STZ method does not produce as a severe insulin deficient state but was adequate to produce hyperglycemia (FIG. 9, FIG. 10 and FIG. 15). Treatment with dual-PEGylated human FGF21 variant (E37C, R77C, P171G) (1 mg/kg) reduced insulin levels by 60% relative to vehicle treatment (FIG. 15) while maintained glucose levels at normal, suggesting the administration of dual-PEGylated human FGF21 variant (E37C, R77C, P171G) improved insulin sensitivity in the MLD STZ-treated mice.

### Effect of the Dual-PEGylated Human FGF21 Variant (E37C, R77C, P171G) on Body Weight:

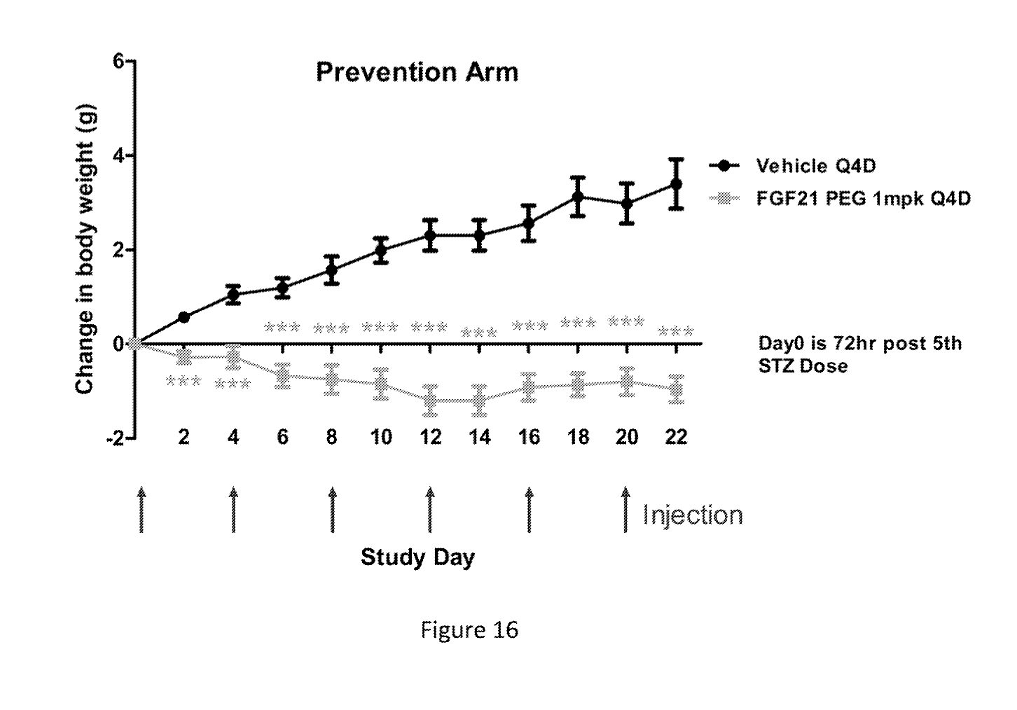

Treatment with the dual-PEGylated human FGF21 variant (E37C, R77C, P171G) (1 mg/kg) caused a sustained reduction of body weight gain in MDL STZ-induced type 1 diabetic mice (FIG. 16). The change in body weight from Day 0 is plotted. By Day 22, the body weight in mice treated with dual-PEGylated human FGF21 variant (E37C, R77C, P171G) was 17% less than vehicle treated mice.

### Example 4

### Effect of the Dual-PEGylated Human FGF21 Variant (E37C, R77C, P171G) in Multiple Low Dose STZ-Induced Type 1 Diabetic Mice (Treatment)

We demonstrated that the dual-PEGylated human FGF21 variant (E37C, R77C, P171G) prevented blood glucose level elevation during T1DM disease progression over the study period of 22 days (Example 3). In that study, the dual-PEGylated human FGF21 variant (E37C, R77C, P171G) was administered three days after the fifth low dose of STZ injection before mice had developed overt type 1 diabetes and hyperglycemia. In the instant study, we evaluate whether FGF21 could reverse hyperglycemia once mice had become over hyperglycemia after MLD STZ injection. The dual-PEGylated human FGF21 variant (E37C, R77C, P171G) was administered 23 days after the fifth dose of STZ injection.

The MLD STZ-mouse model was generated as described in Example 3. Briefly, 7 week old male C57BL6 mice received five consecutive daily intraperitoneal (IP) injections of STZ (Streptozotocin, Sigma S-1030) at 40 mg/kg/day. On Day 21 post the fifth dose of STZ (treatment Day-2), blood glucose levels and body weight were measured and mice were randomized into vehicle or treatment groups. Vehicle (10 mM Tris-HCl, 150 mM NaCl, pH 8.5) or the dual-PEGylated human FGF21 variant (E37C, R77C, P171G) (1 mg/kg) were injected IP into mice on Day 23 post the fifth dose of STZ (treatment Day 0). Compounds were given every 4 days and a total of five IP injections were administered. Blood glucose levels were measured at treatment Day 2, 6, 10, 14, and 18. Body weight was measured 2-3 times per week during the disease induction phase and every other day during the study period. On treatment Day 18, 2 days post the last injection, mice were euthanized and terminal blood samples were collected.

Pancreas from five mice in each group was collected. Histology evaluation and immunohistochemistry for insulin and glucagon was conducted. Pancreas sections of 5 m were deparaffinized and hydrated in deionized H₂O, Sections were blocked with CAS BLOCK (Invitrogen, #00-8120; Camarillo, Calif.) and incubated with rabbit polyclonal anti-glucagon (DAKO #A0565, Carpenteria, Calif.). Slides were quenched with 3% H₂O₂ and followed by Rabbit EnvisionHRP (DAKO #K4003, Carpenteria, Calif.). The reaction sites were visualized with diaminobenzadine (DAB; DAKO #K3468 Carpentaria, Calif.). Sections were then blocked again with CAS BLOCK and incubated with guinea pig polyclonal anti-insulin (DAKO #A0564, Carpenteria, Calif.). Slides were incubated with biotinylated goat anti-guinea pig IgG (Vector #BA7000, Burlingame, Calif.) followed by Vectastain AP-ABC (Vector #AK5000, Burlingame, Calif.). The reaction sites were visualized with AP-Red (Vector #SK5100). The slides were then evaluated microscopically by a pathologist.

### Effect of the Dual-PEGylated Human FGF21 Variant (E37C, R77C, P171G) on Plasma Glucose:

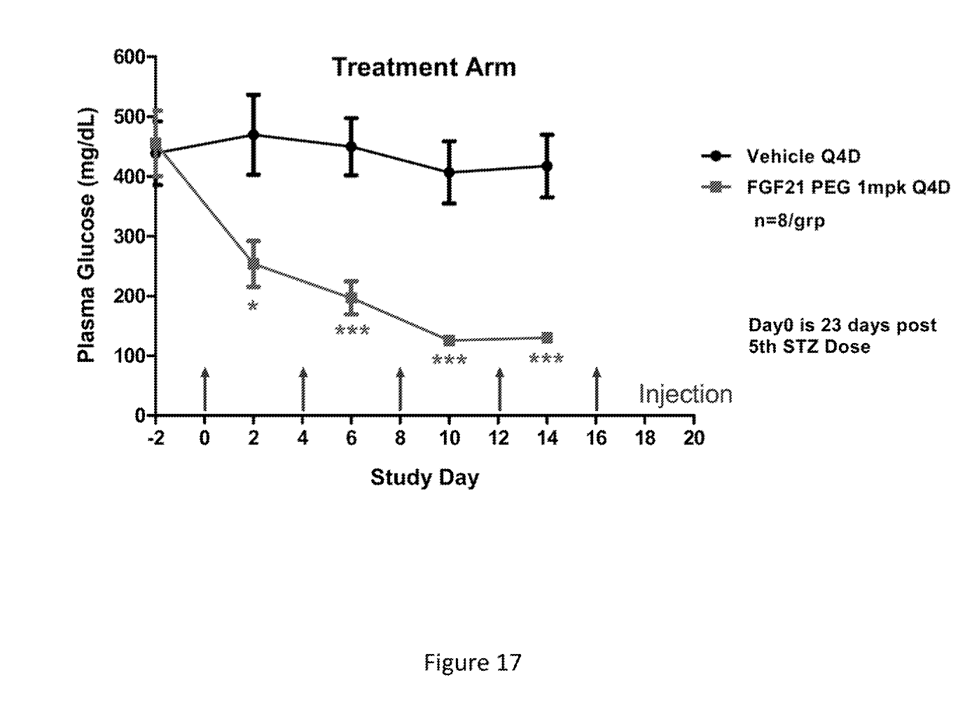

In this study, we investigated the plasma glucose lowering effects of FGF21 administration after mice have become T1DM, 23 days following the MLD STZ induction. Prior to the treatment initiation at 21 days following the MLD STZ induction, the baseline mean blood glucose for both groups was in a range of 438 to 455 mg/dL (treatment Day-2). Vehicle and the dual-PEGylated human FGF21 variant (E37C, R77C, P171G) (1 mg/kg) was administered to mice Q4D. Plasma glucose was subsequently measured every 4 days on days 2, 6, 10, 14, and 18. The dual-PEGylated human FGF21 variant (E37C, R77C, P171G) lowered plasma glucose and ultimately reversed the hyperglycemia in this animal model (FIG. 17). Plasma glucose levels in the dual-PEGylated human FGF21 variant (E37C, R77C, P171G) group were reduced by 46% (Day 2), 56% (day 6), and 69% (day 10, day 14). By study day 10, plasma glucose levels had returned to normoglycemic level.

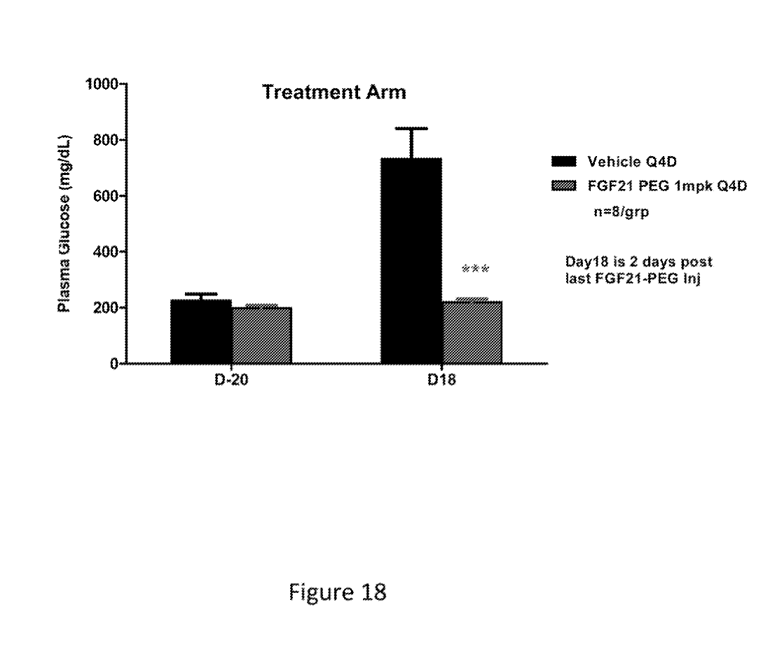

Plasma from blood samples collected on day-20 and 2 days post the last injection on day 18 were run on a clinical chemistry analyzer for a more precise plasma glucose measurement. Similar to blood glucose reductions shown in FIG. 17, treatment with the dual-PEGylated human FGF21 variant (E37C, R77C, P171G) reduced plasma glucose levels to normoglycemic levels. Relative to the vehicle group, the plasma glucose reduction for the dual-PEGylated human FGF21 variant (E37C, R77C, P171G) group was 70% (FIG. 18).

### Effect of the Dual-PEGylated Human FGF21 Variant (E37C, R77C, P171G) on Lipid Levels:

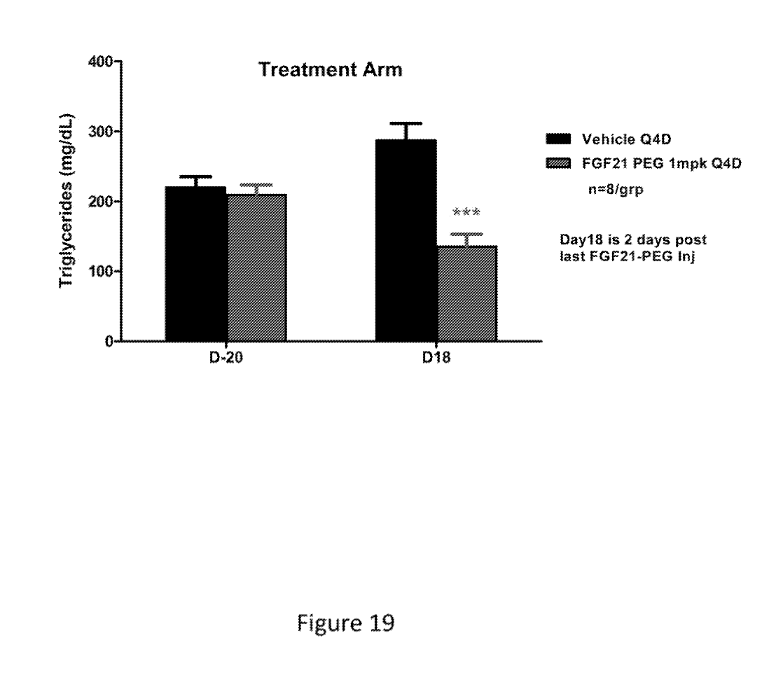

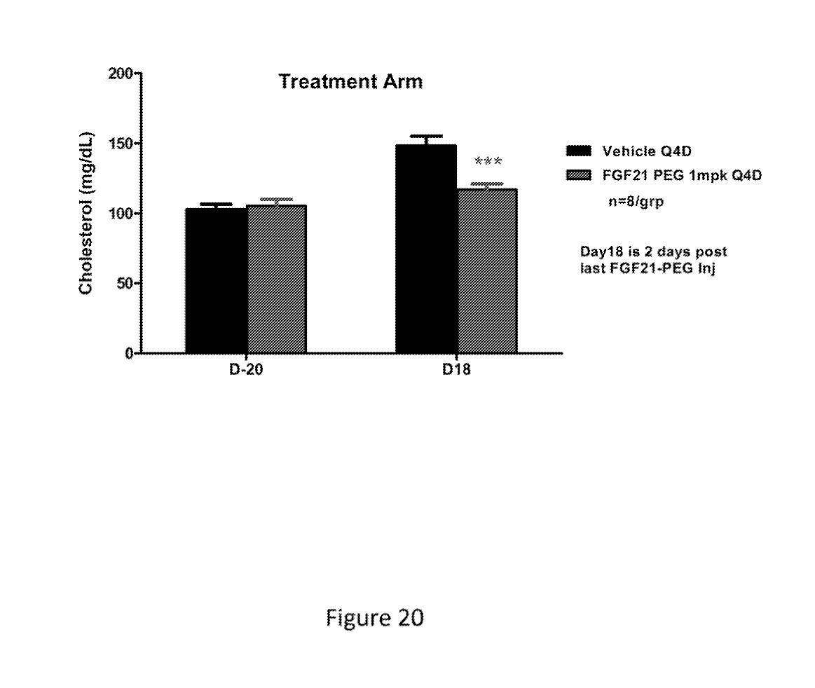

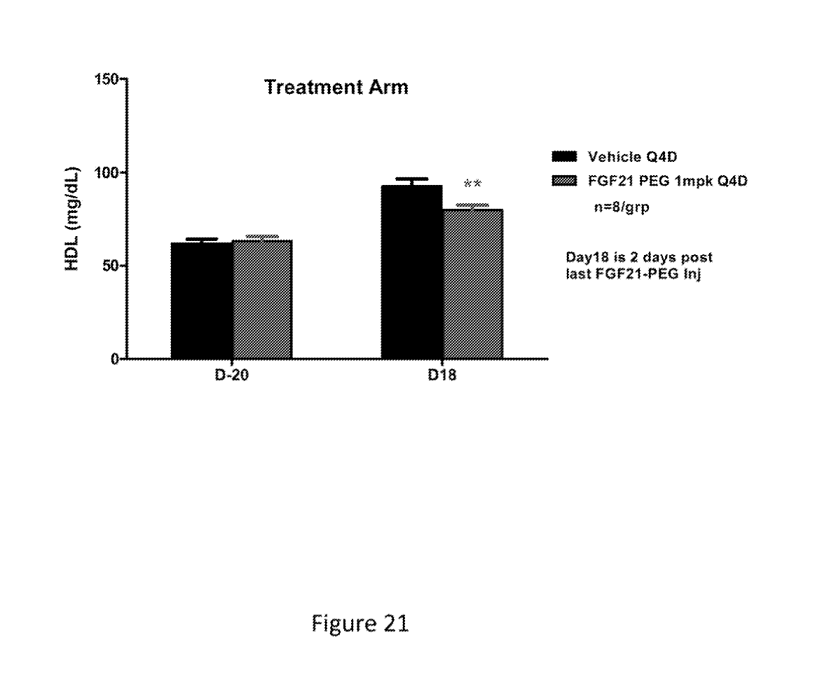

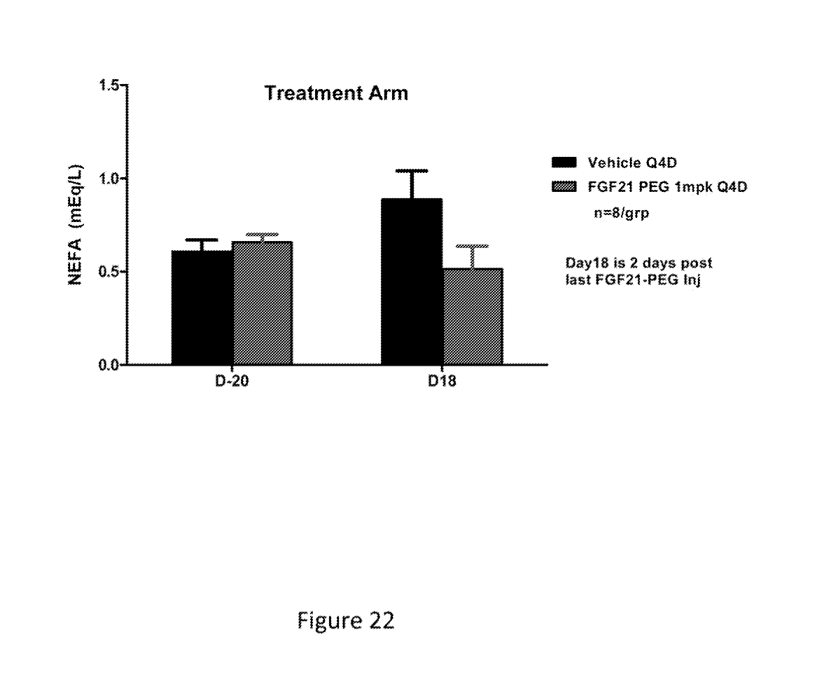

From Day-20 to day 18, triglyceride levels in vehicle treated mice were elevated, while PEG-FGF21 treated mice demonstrated a reduction in triglyceride levels. Relative to vehicle group, plasma triglyceride levels in mice treated with dual-PEGylated human FGF21 variant (E37C, R77C, P171G) were reduced by 53% (FIG. 19). Treatment with FGF21 also lowered total cholesterol, HDL-cholesterol, and NEFA levels by 21%, 14% and 42%, respectively (FIGS. 20, 21, and 22).

### Effect of the Dual-PEGylated Human FGF21 Variant (E37C, R77C, P171G) on Insulin Levels:

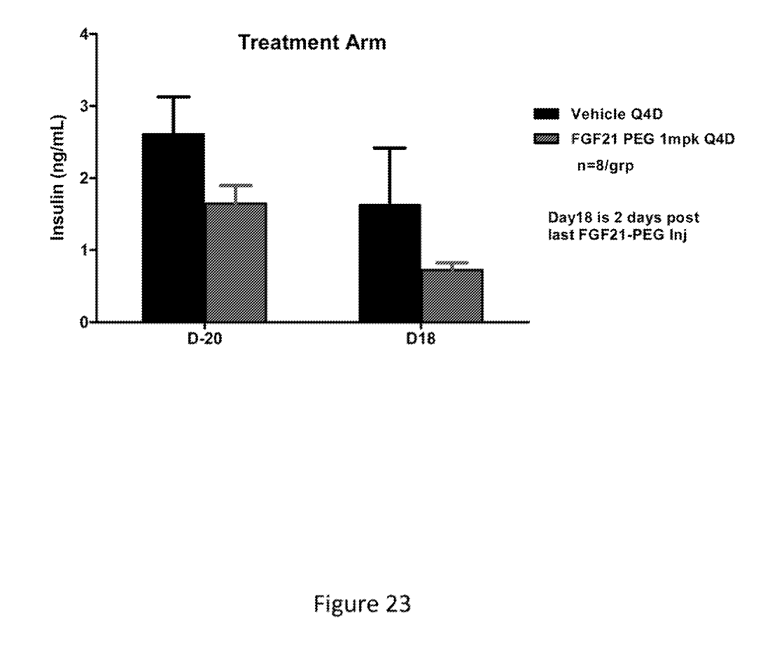

Treatment with the dual-PEGylated human FGF21 variant (E37C, R77C, P171G) reduced insulin levels by 55% relative to vehicle treatment (FIG. 23) while normalized plasma glucose levels (FIGS. 17 & 18), suggesting that administration of the dual-PEGylated human FGF21 variant (E37C, R77C, P171G) improved insulin sensitivity in these mice.

### Effect of the Dual-PEGylated FGF21 Variant on Liver Enzyme Levels:

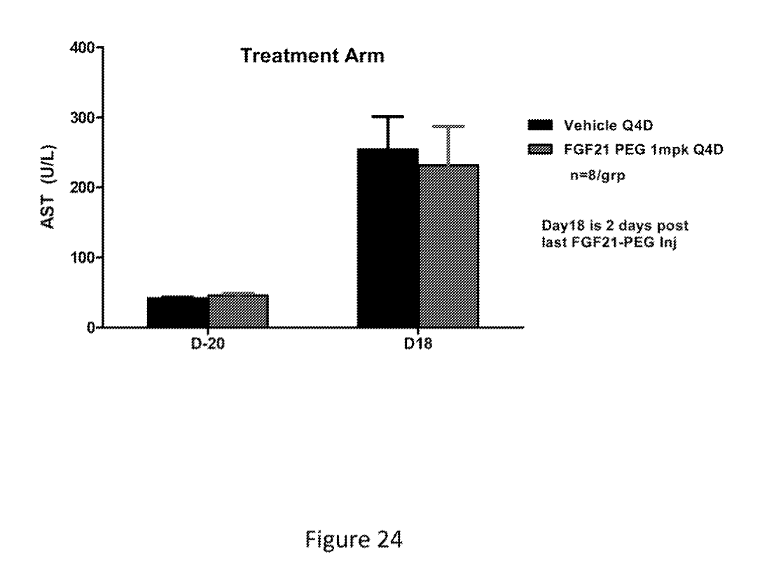

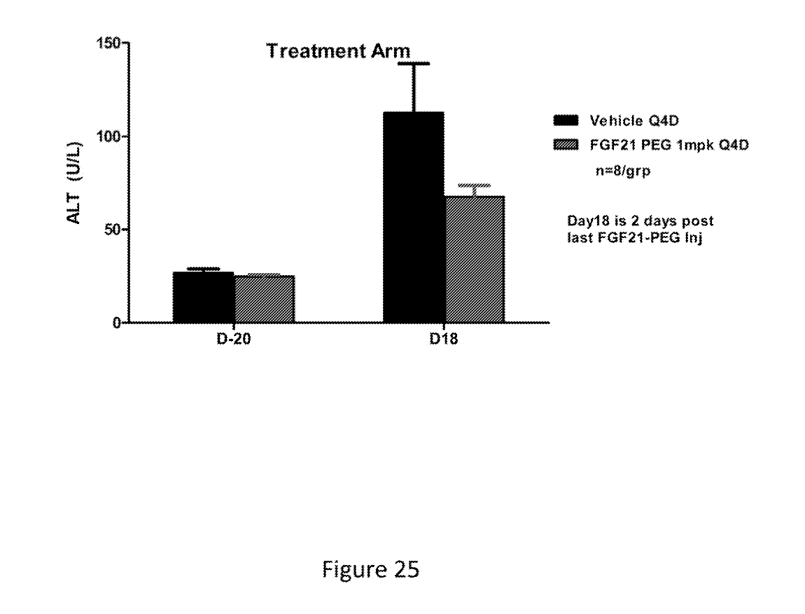

Elevated AST and ALT levels were observed in MLD STZ-treated mice on Day 18 (FIGS. 24 & 25). Mice treated with the dual-PEGylated human FGF21 variant (E37C, R77C, P171G) showed 40% lower ALT levels than vehicle treated mice (FIG. 25).

### Effect of the Dual-PEGylated FGF21 Variant on Body Weights:

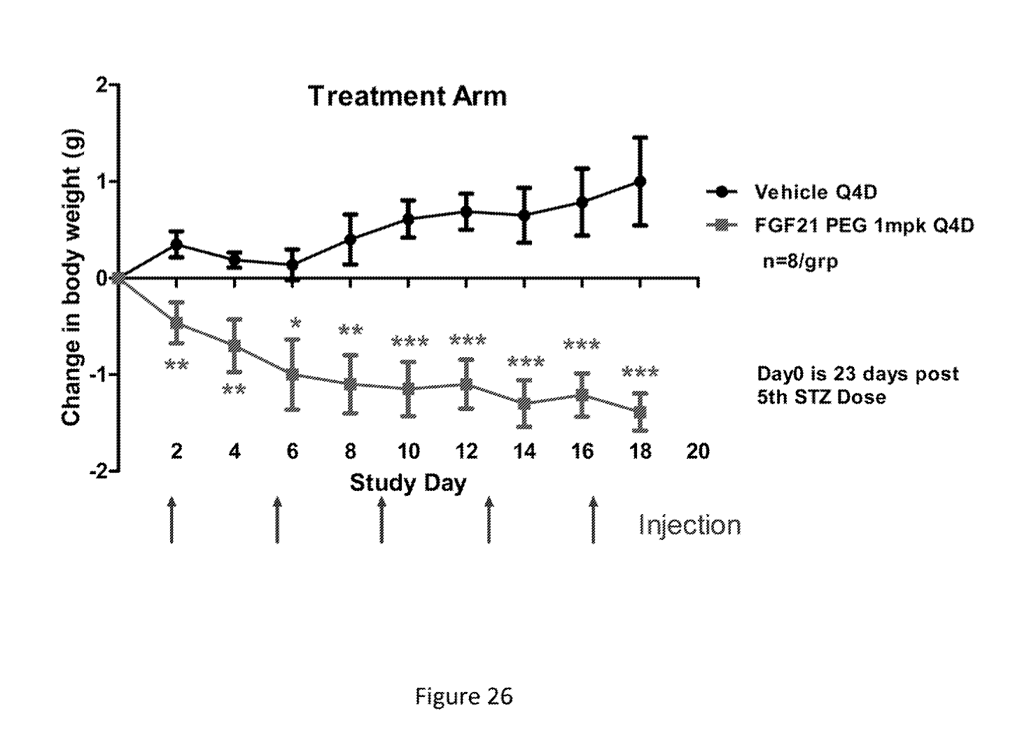

In this study, body weight was progressively reduced by the dual-PEGylated human FGF21 variant (E37C, R77C, P171G) (FIG. 26). By Day 18, the body weight in mice treated with the dual-PEGylated human FGF21 variant (E37C, R77C, P171G) was 6% less than vehicle treated mice.

### Effect of the Dual-PEGylated FGF21 Variant on Beta-Cell Preservation:

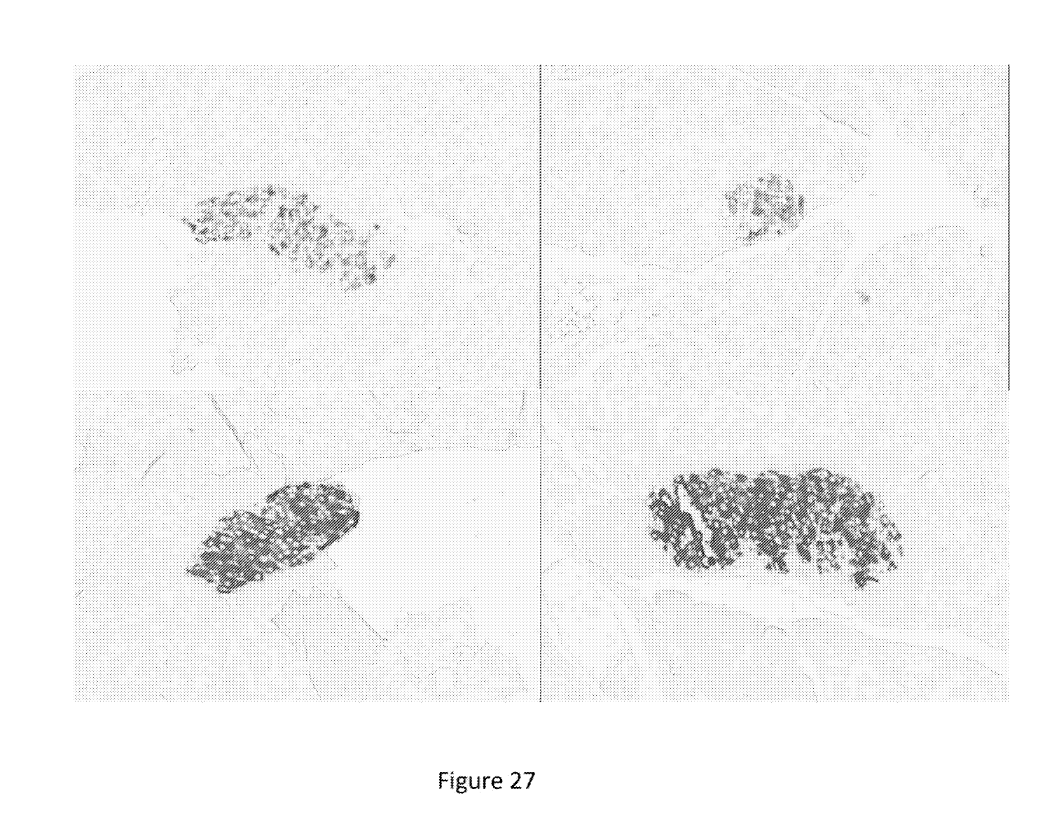

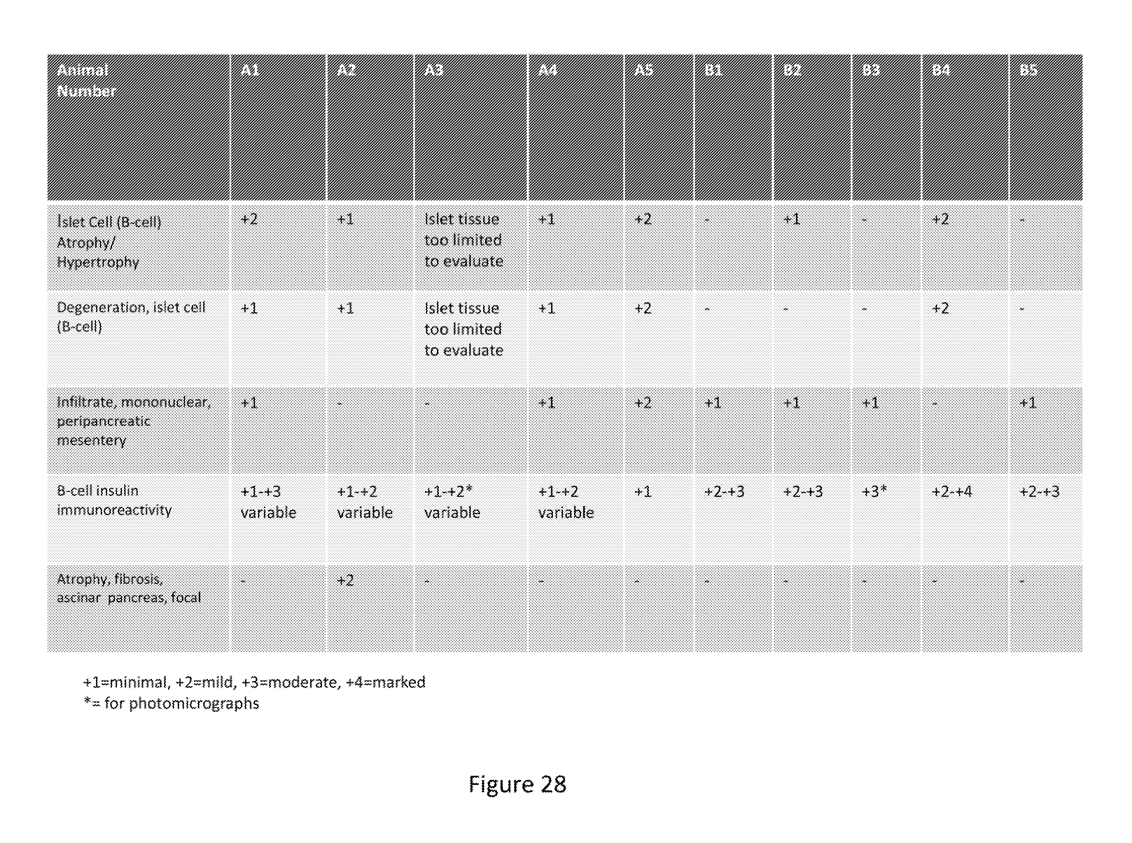

To better understand the beneficial effects of FGF21 administration in STZ-induced T1DM mice, we conducted immunohistochemistry staining for insulin and glucagon and histomorphometric analysis for pancreas. Specifically, beta cell atrophy/hypertrophy, degeneration of islet cells, mononuclear infiltration, and atrophy and fibrosis of surrounding tissues were analyzed. The insulin immunoreactivity of the beta cells is illustrated in FIG. 27. The upper panels are images from a vehicle-treated mouse (mouse A3) and the lower panels are from a mouse treated with the dual-PEGylated human FGF21 variant (E37C, R77C, P171G) (mouse B3). As illustrated, there is increased intensity and uniformity of insulin immunoreactivity in the mouse treated with the dual-PEGylated human FGF21 variant (E37C, R77C, P171G). FIG. 28 summarizes the insulin immunoreactivity and morphometric findings from each vehicle and the mouse treated with the dual-PEGylated human FGF21 variant (E37C, R77C, P171G). Vehicle-treated mice are denoted A1 through A5, while mice treated with the dual-PEGylated human FGF21 variant (E37C, R77C, P171G) are denoted as B1 through B5. In summary, nearly all vehicle-treated mice demonstrated some islet cell atrophy/hypertrophy and degeneration, while only 1-2 mice in the group treated with the dual-PEGylated human FGF21 variant (E37C, R77C, P171G) demonstrated these effects. The fact that insulin immunoreactivity was profoundly decreased in vehicle-treated mice also suggests that a greater percentage of beta cells were destroyed in vehicle-treated mice than those treated with the dual-PEGylated human FGF21 variant (E37C, R77C, P171G). Overall, these morphometric results confirm that FGF21 treatment not only lowers glucose and lipid levels, but also demonstrates some protective effects on beta cells from further immunological destruction and progression of T1DM.

### CONCLUSIONS

Collectively, the data presented in Examples 1-4 indicate that FGF21 presents a new therapeutic option for Type 1 diabetes patients. FGF21 treatment alone was sufficient to reduce plasma glucose and lipid levels in both high and low dose STZ-induced type 1 diabetic rodent models. In addition, FGF21 treatment in conjunction with insulin treatment provides additive plasma glucose lowering effect. PEGylation of the human FGF21 variant (E37C, R77C, P171G) dramatically extended the plasma glucose lowering effect up to 7 days post a single injection. Chronic administration of this molecule not only prevented the progression of T1DM but also reversed the plasma glucose and lipid level elevations in T1DM mice. From our morphometric analysis, we further demonstrated that FGF21 administration increased islet insulin contents and protected beta cells from destruction. This may offer a mechanistic explanation for the beneficial effect of FGF21 observed in T1DM animal model. Overall, we provided evidence that FGF21 has potential for the treatment of type 1 diabetes.

What is claimed is:

1. A method of treating a metabolic disorder comprising administering to a subject in need thereof a therapeutically effective amount of (a) an isolated human FGF21 polypeptide; or (b) an FGF21 variant polypeptide.

2. The method of claim 1, wherein the metabolic disorder is type 1 diabetes.

3. The method of claim 1, wherein the metabolic disorder is dyslipidemia.

4. The method of claim 1, wherein the metabolic disorder is obesity.

5. The method of claim 1, wherein the metabolic disorder is diabetic nephropathy.

6. The method of claim 1, wherein the metabolic disorder comprises a condition in which the subject has a fasting blood glucose level of greater than or equal to 100 mg/dL.

7. The method of claim 1, wherein the subject is a mammal.

8. The method of claim 7, wherein the mammal is a human.

9. The method of claim 1, wherein the human FGF21 polypeptide comprises one of SEQ ID NOs:4 and 8.

10. The method of claim 1, wherein the human FGF21 polypeptide is encoded by one of SEQ ID NOs:3 and 7.

11. The method of claim 1, wherein the FGF21 variant comprises one or more mutations in the mature FGF21 sequence of SEQ ID NO:4 or SEQ ID NO:8 selected from the mutations presented in Tables 1-13.

12. The method of claim 1, wherein the FGF21 polypeptide is administered in the form of a pharmaceutical composition comprising the FGF21 polypeptide in admixture with a pharmaceutically-acceptable carrier.

13. The method of claim 1, further comprising the step of determining the subject's blood glucose level at a timepoint subsequent to the administration.

14. The method of claim 1, further comprising the step of determining the subject's serum insulin level at a timepoint subsequent to the administration.

15. The method of claim 1, wherein the human FGF21 polypeptide or human FGF21 variant polypeptide further comprises one or more of
(a) one or more PEG molecules; and
(b) an Fc polypeptide.

16. A method of treating a metabolic disorder comprising administering to a subject in need thereof a therapeutically effective amount of a human FGF21 polypeptide comprising an amino acid sequence that has at least 90% sequence identity with one of SEQ ID NOs:4 and 8.

17. The method of claim 16, wherein the metabolic disorder is type 1 diabetes.

18. The method of claim 16, wherein the metabolic disorder is dyslipidemia.

19. The method of claim 16, wherein the metabolic disorder is obesity.

20. The method of claim 16, wherein the metabolic disorder is diabetic nephropathy.

21. The method of claim 16, wherein the metabolic disorder comprises a condition in which the subject has a fasting blood glucose level of greater than or equal to 100 mg/dL.

22. The method of claim 16, wherein the subject is a mammal.

23. The method of claim 21, wherein the mammal is a human.

24. The method of claim 16, wherein the human FGF21 polypeptide is administered in the form of a pharmaceutical composition comprising the human FGF21 polypeptide in admixture with a pharmaceutically-acceptable carrier.

25. The method of claim 16, further comprising the step of determining the subject's blood glucose level at a timepoint subsequent to the administration.

26. The method of claim 25, further comprising the step of determining the subject's serum insulin level at a timepoint subsequent to the administration.

27. The method of claim 16, wherein the FGF21 polypeptide comprises one or more mutations in the mature FGF21 sequence of SEQ ID NO:4 or 8 selected from the mutations presented in Tables 1-13.

28. The method of claim 16, wherein the FGF21 polypeptide further comprises one or more of
(a) one or more PEG molecules; and
(b) an Fc polypeptide.

29. The method of claim 1, wherein the isolated human FGF21 polypeptide or FGF21 variant polypeptide comprises one of SEQ ID NOs:10 and 12.

30. The method of claim 29, wherein the isolated human FGF21 polypeptide; or FGF21 variant polypeptide comprises one of SEQ ID NOs:39 and 41.


SEQUENCE LISTING (47 sequences)

SEQ ID NO: 1 - DNA, 630 bp, Homo sapiens
  CDS (1)..(630)
atggactcggacgagaccgggttcgagcactcaggactgtgggtttctgtgctggctggtcttctgctgggagcctgccaggcacaccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcacccccggagccacccggaatcctggccccccagccccccgatgtgggctcctcggaccctctgagcatggtgggaccttcccagggccgaagccccagctacgcttcctga

SEQ ID NO: 2 - protein, 209 aa, Homo sapiens [sequence omitted]

SEQ ID NO: 3 - DNA, 546 bp, Homo sapiens
  CDS (1)..(546)
caccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcacccccggagccacccggaatcctggccccccagccccccgatgtgggctcctcggaccctctgagcatggtgggaccttcccagggccgaagccccagctacgcttcctga

SEQ ID NO: 4 - protein, 181 aa, Homo sapiens
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 5 - DNA, 630 bp, Homo sapiens
  CDS (1)..(630)
atggactcggacgagaccgggttcgagcactcaggactgtgggtttctgtgctggctggtcttctgctgggagcctgccaggcacaccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcactcccggagccacccggaatcctggccccccagccccccgatgtgggctcctcggaccctctgagcatggtgggaccttcccagggccgaagccccagctacgcttcctga

SEQ ID NO: 6 - protein, 209 aa, Homo sapiens
MDSDETGFEHSGLWVSVLAGLLLGACQAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 7 - DNA, 546 bp, Homo sapiens
  CDS (1)..(546)
caccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcactcccggagccacccggaatcctggccccccagccccccgatgtgggctcctcggaccctctgagcatggtgggaccttcccagggccgaagccccagctacgcttcctga

SEQ ID NO: 8 - protein, 181 aa, Homo sapiens
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 9 - DNA, 543 bp, Artificial Sequence
  Human FGF21 L98R P171G
catccaattccagattcttctccattattacaattcgggggccaagtccggcagcgttacctctacacagatgatgcccagcagacagaagcccacctggagatcagggaggacgggacggtggggggtgctgctgaccagagccccgaaagtctcctgcagctgaaagccttgaagccgggtgttattcaaatcttgggtgtcaagacatccaggttcctgtgccagcggccagatggggccctgtatggatcgctccactttgaccctgaggcctgcagcttccgtgagcgtcttcttgaggacggttacaatgtttaccagtccgaagcccacggcctcccgctgcacctgccagggaacaagtccccacaccgtgaccctgcaccccgaggaccagctcgcttcctgccactaccaggcctgccccccgcacccccggagccacccggaatcctggccccccagccccccgatgtgggctcctcggaccctctgagcatggtgggtggttcccagggccgaagccccagctacgcttcc

SEQ ID NO: 10 - protein, 181 aa, Artificial Sequence
  Synthetic Construct
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPAPPEPPGILAPQPPDVGSSDPLSMVGGSQGRSPSYAS

SEQ ID NO: 11 - DNA, 543 bp, Artificial Sequence
  Human FGF21 L98R P171G A180E Variant
catccaattccagattcttctccattattacaattcgggggccaagtccggcagcgttacctctacacagatgatgcccagcagacagaagcccacctggagatcagggaggacgggacggtggggggtgctgctgaccagagccccgaaagtctcctgcagctgaaagccttgaagccgggtgttattcaaatcttgggtgtcaagacatccaggttcctgtgccagcggccagatggggccctgtatggatcgctccactttgaccctgaggcctgcagcttccgtgagcgtcttcttgaggacggttacaatgtttaccagtccgaagcccacggcctcccgctgcacctgccagggaacaagtccccacaccgtgaccctgcaccccgaggaccagctcgcttcctgccactaccaggcctgccccccgcacccccggagccacccggaatcctggccccccagccccccgatgtgggctcctcggaccctctgagcatggtgggtggttcccagggccgaagccccagctacgaatcc

SEQ ID NO: 12 - protein, 181 aa, Artificial Sequence
  Synthetic Construct
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPAPPEPPGILAPQPPDVGSSDPLSMVGGSQGRSPSYES

SEQ ID NO: 13 - protein, 4 aa, Artificial
  G4 Linker
GGGG

SEQ ID NO: 14 - protein, 5 aa, Artificial
  G5 Linker
GGGGG

SEQ ID NO: 15 - protein, 5 aa, Artificial Sequence
  synthesized linker
GGGGS

SEQ ID NO: 16 - protein, 10 aa, Artificial Sequence
  (G4S)2 Linker
GGGGSGGGGS

SEQ ID NO: 17 - protein, 25 aa, Artificial Sequence
  synthesized linker
GGGGSGGGGSGGGGSGGGGSGGGGS

SEQ ID NO: 18 - protein, 6 aa, Artificial Sequence
  synthesized linker
GGEGGG

SEQ ID NO: 19 - protein, 8 aa, Artificial Sequence
  synthesized linker
GGEEEGGG

SEQ ID NO: 20 - protein, 5 aa, Artificial Sequence
  synthesized linker
GEEEG

SEQ ID NO: 21 - protein, 4 aa, Artificial Sequence
  synthesized linker
GEEE

SEQ ID NO: 22 - protein, 6 aa, Artificial Sequence
  synthesized linker
GGDGGG

SEQ ID NO: 23 - protein, 7 aa, Artificial Sequence
  synthesized linker
GGDDDGG

SEQ ID NO: 24 - protein, 5 aa, Artificial Sequence
  synthesized linker
GDDDG

SEQ ID NO: 25 - protein, 4 aa, Artificial Sequence
  synthesized linker
GDDD

SEQ ID NO: 26 - protein, 21 aa, Artificial Sequence
  synthesized linker
GGGGSDDSDEGSDGEDGGGGS

SEQ ID NO: 27 - protein, 5 aa, Artificial Sequence
  synthesized linker
WEWEW

SEQ ID NO: 28 - protein, 5 aa, Artificial Sequence
  synthesized linker
FEFEF

SEQ ID NO: 29 - protein, 6 aa, Artificial Sequence
  synthesized linker
EEEWWW

SEQ ID NO: 30 - protein, 6 aa, Artificial Sequence
  synthesized linker
EEEFFF

SEQ ID NO: 31 - protein, 7 aa, Artificial Sequence
  synthesized linker
WWEEEWW

SEQ ID NO: 32 - protein, 7 aa, Artificial Sequence
  synthesized linker
FFEEEFF

SEQ ID NO: 33 - protein, 15 aa, Artificial
  (G4S)3 Linker
GGGGSGGGGSGGGGS

SEQ ID NO: 34 - protein, 8 aa, Artificial
  G3KG4 linker
GGGKGGGG

SEQ ID NO: 35 - protein, 8 aa, Artificial
  G3NGSG2 Linker
GGGNGSGG

SEQ ID NO: 36 - protein, 8 aa, Artificial
  G3CG4 linker
GGGCGGGG

SEQ ID NO: 37 - protein, 5 aa, Artificial
  GPNG2 Linker
GPNGG

SEQ ID NO: 38 - DNA, 1275 bp, Artificial Sequence
  Fc-(G4S)3-RG
atggacaaaactcacacatgtccaccttgtccagctccggaactcctggggggaccgtcagtcttcctcttccccccaaaacccaaggacaccctcatgatctcccgtacccctgaggtcacatgcgtggtggtggacgtgagccacgaagaccctgaggtcaagttcaactggtacgtggacggcgtggaggtgcataatgccaagacaaagccgcgtgaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgtgatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagccgttggcagcaggggaacgtcttctcatgctccgtgatgcatgaggctctgcacaaccactacacgcagaagagcctctccctgtctccgggtaaaggtggtggtggttccggtggcggcggctctggtggtggtggcagccatccgatcccggactcttctccgctgctgcagttcggtggtcaggttcgtcagcgttacctgtacaccgacgacgcgcagcagaccgaggcgcacctggagatccgtgaagacggtaccgttggtggtgcggccgaccagtctccggaatctctgctgcagctgaaagccctgaaaccgggtgttatccagatcctgggcgttaaaacctctcgtttcctgtgccagcgtccggacggcgccctgtacggttctctgcacttcgacccggaggcgtgctcttttcgtgaacgtctgctcgaagacggttacaacgtttaccagtctgaggcgcacggtctgccgctgcacctgccgggtaacaaatctccgcaccgtgacccggcgccacgtggtcctgcgcgtttcctgccactgccgggcctgccgcctgcgcctcctgaaccgcctggtatcctggctccgcagccgccagacgttggttcttctgacccgctgtctatggttggtggctctcagggtcgttctccgtcttacgcctcttaa

SEQ ID NO: 39 - protein, 424 aa, Artificial Sequence
  Synthetic Construct
MDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPAPPEPPGILAPQPPDVGSSDPLSMVGGSQGRSPSYAS

SEQ ID NO: 40 - DNA, 1275 bp, Artificial
  Fc-(G4S)3-RGE
atggacaaaactcacacatgtccaccttgtccagctccggaactcctggggggaccgtcagtcttcctcttccccccaaaacccaaggacaccctcatgatctcccgtacccctgaggtcacatgcgtggtggtggacgtgagccacgaagaccctgaggtcaagttcaactggtacgtggacggcgtggaggtgcataatgccaagacaaagccgcgtgaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgtgatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagccgttggcagcaggggaacgtcttctcatgctccgtgatgcatgaggctctgcacaaccactacacgcagaagagcctctccctgtctccgggtaaaggtggtggtggttctggtggtggtggtagcggtggtggtggttcccatccaattccagattcttctccattattacaattcgggggccaagtccggcagcgttacctctacacagatgatgcccagcagacagaagcccacctggagatcagggaggacgggacggtggggggtgctgctgaccagagccccgaaagtctcctgcagctgaaagccttgaagccgggtgttattcaaatcttgggtgtcaagacatccaggttcctgtgccagcggccagatggggccctgtatggatcgctccactttgaccctgaggcctgcagcttccgtgagcgtcttcttgaggacggttacaatgtttaccagtccgaagcccacggcctcccgctgcacctgccagggaacaagtccccacaccgtgaccctgcaccccgaggaccagctcgcttcctgccactaccaggcctgccccccgcacccccggagccacccggaatcctggccccccagccccccgatgtgggctcctcggaccctctgagcatggtgggtggttcccagggccgaagccccagctacgaatcctaa

SEQ ID NO: 41 - protein, 424 aa, Artificial
  Synthetic Construct
MDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPAPPEPPGILAPQPPDVGSSDPLSMVGGSQGRSPSYES

SEQ ID NO: 42 - protein, 20 aa, Artificial Sequence
  (G4S)4 Linker
GGGGSGGGGSGGGGSGGGGS

SEQ ID NO: 43 - protein, 5 aa, Artificial Sequence
  Artificial Peptidyl Linker Sequence
XXYXX

SEQ ID NO: 44 - protein, 6 aa, Artificial Sequence
  Artificial Peptidyl Linker Sequence
XXSXXG

SEQ ID NO: 45 - protein, 6 aa, Artificial Sequence
  Artificial Peptidyl Linker
XXTXXG

SEQ ID NO: 46 - protein, 6 aa, Artificial Sequence
  XXNXXG Linker Sequence
XXNXXG

SEQ ID NO: 47 - protein, 230 aa, Artificial Sequence
  IgG4 Fc
XESKYGPPCPXCPAPXXXGGPSVFLFPPKPKDTLMISRTPEVTCVVVDVSQEDPEVQFNWYVDGVEVHNAKTKPREEQFXSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGX